(19) United States
(12) Patent Application Publication
(10) Pub. No.: US 2023/0293539 A1
(43) Pub. Date: Sep. 21, 2023

(54) MLL1-WDR5 PROTEIN-PROTEIN INTERACTION INHIBITOR COMPOUNDS AND USES THEREOF

(71) Applicant: HUYABIO International, LLC, San Diego, CA (US)

(72) Inventors: Farbod SHOJAEI, San Diego, CA (US); Che FANG, Lake Forest, CA (US); J. Edward SEMPLE, San Diego, CA (US); Mireille GILLINGS, San Diego, CA (US)

(21) Appl. No.: 18/120,311

(22) Filed: Mar. 10, 2023

Related U.S. Application Data
(60) Provisional application No. 63/319,589, filed on Mar. 14, 2022.

(51) Int. Cl. A61K 31/5377; A61K 31/496; A61P 35/00; A61P 25/00

(52) U.S. Cl. CPC A61K 31/5377; A61K 31/496; A61P 35/00; A61P 25/00

(57) ABSTRACT

Described herein are phenyl triazole and aniline compounds that are MLL1-WDR5 protein-protein interaction inhibitors. Also disclosed herein are pharmaceutical compositions and methods of use for the phenyl triazole and the aniline compounds.

### CROSS-REFERENCE

This application claims the benefit of priority from U.S. Provisional Application No. 63/319,589, filed Mar. 14, 2022, the contents of which are incorporated herein by reference in their entirety.

### FIELD OF THE DISCLOSURE

The present invention relates to the field of pharmaceutical chemistry, and more particularly to phenyl triazole and aniline compounds that inhibit MLL1-WDR5 protein-protein interactions, and preparation and medical uses thereof.

### BACKGROUND OF THE DISCLOSURE

Translocation and re-arrangement of the methyl transferase mixed lineage leukemia 1 (MLL1) gene for histone H3K4 can lead to mixed lineage leukemia (including acute myeloid leukemia and acute lymphoid leukemia). After re-arrangement, the MLL1 gene fuses with other chaperone genes to form fusion genes, and the carcinogenic MLL fusion protein is expressed. The MLL fusion protein can interact with RNA polymerase II (Pol II) related elongation factors to form the super elongation complex (SEC). The SEC complex can lead to abnormal expression of the Hox gene regulated by MLL1 gene, which causes a series of serious consequences including onset of MLL1 leukemia.

The C-terminal WIN motif moiety of MLL protein is capable of binding WDR5, RbBP5, Ash2L and DPY30 to form complexes. MLL1 interacts with WDR5 directly through the C-terminal WIN motif moiety, to mediate the interaction between the catalytic domain of MLL1 and other protein complexes. When WDR5 is knocked out, the level of H3K4me2/3 decreases and the Hox gene expression is downregulated to induce the apoptosis of leukemia cells. Therefore, specific inhibition of the enzymatic activity of the wildtype MLL1 can achieve the effect of treating leukemia.

Thus, use of small molecule inhibitors to interfere with the protein-protein interaction of MLL1-WDR5 is an effective method to inhibit MLL1 enzymatic activity and downregulate Hox and Meis-1 gene expression to block the progression of leukemia. MLL1-WDR5 protein-protein interaction inhibitors have been described in WO2019205687A1 and WO2020172932A1, each of which is herein incorporated by reference in its entirety. A need exists for additional MLL1-WDR5 protein-protein interaction inhibitors having additional and/or additional improved properties.

### SUMMARY

The foregoing and additional needs are satisfied by embodiments described herein. Provided herein are small molecule compounds that can regulate MLL1-WDR5 protein-protein interaction, and compositions and methods of using the compounds and compositions. Inhibition of MLL1-WDR5 protein-protein interactions inhibits the enzyme catalytic activity of MLL1, downregulate the methylation level of H3K4 and the gene expression levels of Hox and Meis-1 genes to induce the apoptosis of leukemia cells. Therefore, the compound and compositions described herein can be used to treat cancers such as, but not limited to, leukemia.

Some embodiments described herein are phenyl triazole and aniline compounds, or pharmaceutically acceptable salts or solvates thereof. In some embodiments, the compounds are those described in Table 1, hereinbelow.

Some embodiments described herein are pharmaceutical compositions including a compound as described herein, or a pharmaceutically acceptable salt or solvate thereof, and one or more pharmaceutically acceptable carriers, diluents and excipients. In some embodiments provided herein, there is described a pharmaceutical composition comprising the compound of Formula (I), Formula (II), Formula (III), Formula (Ia), Formula (Ib), Formula (IIIa), or Formula (IIIb), or a pharmaceutically acceptable salt thereof, and optionally one or more pharmaceutically acceptable ingredients. Some embodiments described herein provide a process of making a pharmaceutical composition comprising a compound or a pharmaceutically acceptable salt and admixing the compound with one or more pharmaceutically acceptable ingredients. Thus, some Some embodiments described herein, there is provided a use of any one or more of the compounds of Formula (I), Formula (II), Formula (III), Formula (Ia), Formula (Ib), Formula (IIIa), or Formula (IIIb), or a pharmaceutically acceptable salt thereof, and optionally one or more pharmaceutically acceptable ingredients for preparation of a pharmaceutical composition. In some embodiments, the pharmaceutical composition comprises one or more pharmaceutically acceptable ingredients.

Some embodiments herein are methods for the treatment or prevention of cancer in a patient in need thereof, including administering to the patient a therapeutically acceptable dose of the compound described herein, or a pharmaceutically acceptable salt or solvate thereof. Some embodiments provided herein are methods for the treatment or prevention of acute leukemia in a patient in need thereof, including administering to the patient a therapeutically acceptable dose of the compound described herein, or a pharmaceutically acceptable salt or solvate thereof. Some embodiments provided herein are methods for the treatment or prevention of acute leukemia in a patient in need thereof, including administering to the patient a pharmaceutical composition as described herein. In some embodiments, the acute leukemia treated is acute leukemia with MLL1 gene rearrangement.

In some embodiments there is provided herein a method for treating cancer, the method comprising administering to a patient in need thereof a composition comprising an effective amount of a compound having a structure of Formula (I) or Formula (III):

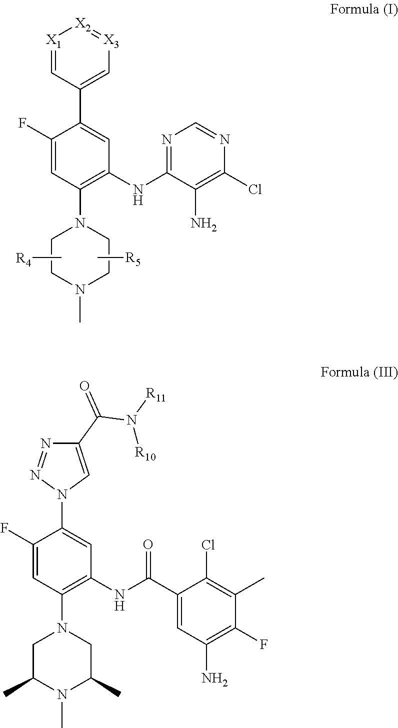

wherein:
X₁ is N or CH;
X₂ is N, CR₂;
X₃ is N or CH;
R₂ is selected from N-morpholino, wherein the morpholino group may be substituted by one or two methyl groups;
each of R₄ and R₅ is hydrogen or alkyl, wherein one or both of R₄ and R₅ may be alkyl; and each of R₁₀ and R₁₁ is C₁-C₄ alkyl or R₁₀ and R₁₁ together form a 4-alkyl piperazinyl group.

In some embodiments of the methods method for treating cancer disclosed herein, the compound has a structure selected from the group consisting of a Formula (Ia), Formula (Ib), Formula (IIIa) and Formula (IIIb), or a pharmaceutically acceptable salt, polymorph, solvate, enantiomer, stereoisomer, or prodrug thereof:

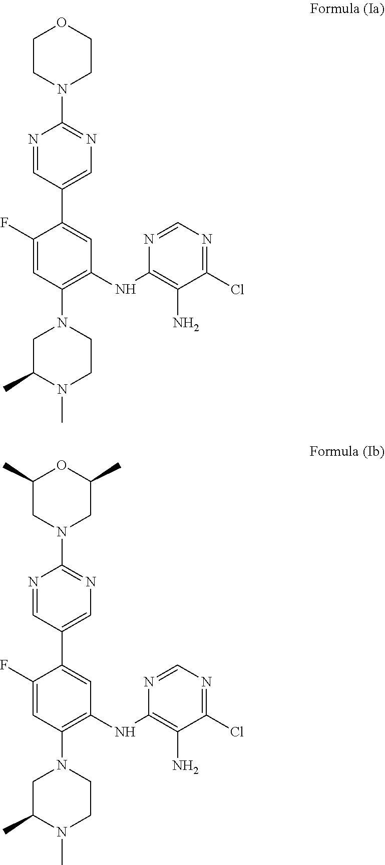

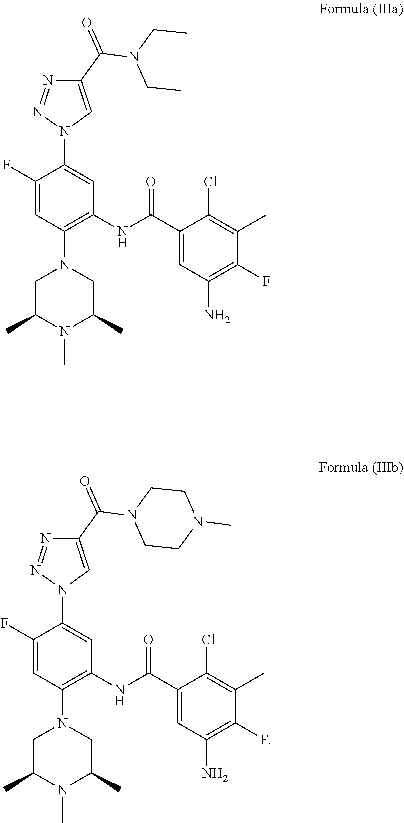 [SMILES: CCN(CC)C(=O)c1cn(-c2cc(NC(=O)c3cc(N)c(F)c(C)c3Cl)c(N3C[C@@H](C)N(C)[C@@H](C)C3)cc2F)nn1 ; Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

In some embodiments of the methods of treating cancer, the compound has a structure of Formula (Ia), or a pharmaceutically acceptable salt, polymorph, solvate, enantiomer, stereoisomer, or prodrug thereof. In some embodiments, the compound has a structure of Formula (Ib), or a pharmaceutically acceptable salt, polymorph, solvate, enantiomer, stereoisomer, or prodrug thereof. In some embodiments, the compound has a structure of Formula (IIIa), or a pharmaceutically acceptable salt, polymorph, solvate, enantiomer, stereoisomer, or prodrug thereof. In some embodiments, the compound has a structure of Formula (IIIb), or a pharmaceutically acceptable salt, polymorph, solvate, enantiomer, stereoisomer, or prodrug thereof. In some embodiments, the compound has a structure of Formula (II) or a pharmaceutically acceptable salt, polymorph, solvate, enantiomer, stereoisomer, or prodrug thereof:

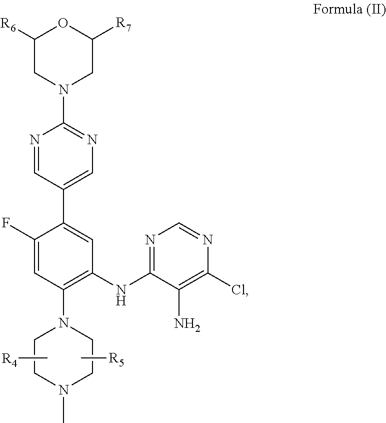

wherein:
each of R₄ and R₅ is hydrogen or methyl, wherein at least one of R₅ and R₆ is methyl, and
each of R₆ and R₇ is hydrogen or methyl.

In some embodiments of the methods of treating cancer herein, the method comprises oral administration of an effective amount of the compound to the patient. In some embodiments, the effective amount of the compound is about 0.1 to about 1000 mg per kg of the patient's body weight (mpk). In some embodiments, the effective amount of the compound is about 1 to about 100 mg per kg of the patient's body weight (mpk). In some embodiments, the effective amount of the compound is about 1 to about 20 mg per kg of the patient's body weight (mpk). In some embodiments, the patient is a human and the compound is administered at a dose of about 1 to about 20 mg per kg of the patient's body weight. In some embodiments, the compound is administered to a human at a dose of about 1 to about 20 mg per kg of the patient's body weight, a brain concentration Cₘₐₓ of about 10 to about 310 ng per gram of the patient's estimated brain weight. In some embodiments, the method of treating cancer comprises administering the compound once per day (Q.D.), once every other day (Q.O.D.), every week (Q.W.), two times per week (BIW) or three times per week (TIW). In some embodiments, the method of treating cancer comprises administering the compound on a dosing schedule, wherein the dosing schedule comprises: (1) a first dosing period comprising from about one cycle of administration of the effective dose of the compound at a frequency of from about once per week (Q.W.); (2) a first drug holiday; and (3) a second dosing period comprising from about one cycle of administration of the effective dose of the compound at a frequency of from about once per week (Q.W.). In some embodiments, each cycle is from about seven days or from about twelve days long. In some embodiments, the drug holiday is from about as long as each cycle. In some embodiments, the method of treating cancers comprises administering the compound on a dosing schedule, wherein the dosing schedule comprises: (1) a first dosing period comprising from about one cycle of administration of the effective dose of the compound at a frequency of from about once per week (Q.W.); and (2) a first drug holiday. In some embodiments, the drug holiday is from about as long as about one cycle. The drug holiday is from about one month, one to six months, or one to twelve months.

In some embodiments of the methods of treating cancer with a compound of Formula (I), Formula (II), Formula (III), Formula (Ia), Formula (Ib), Formula (IIIa), or Formula (IIIb), the compound, when administered orally to a dog in an amount of 1.5 mg per kilogram of the dog's body weight (mpk), results in a maximum blood plasma concentration (Cmax) in the dog of from about 150 to about 235 nM. In some embodiments, the compound, when administered orally to a dog in an amount of 1.5 mg per kilogram of the dog's body weight (mpk), the area under the area under the blood plasma concentration curve (AUC0-last) is from about 4225 nM·h to about 6603 nM·h. In some embodiments, the compound, when administered orally to a dog in an amount of 4.5 mg per kilogram of the dog's body weight (mpk), results in a maximum blood plasma concentration (Cmax) in the dog of from about 356 nM to about 556 nM. In some embodiments, the compound, when administered orally to a dog in an amount of 4.5 mg per kilogram of the dog's body weight (mpk), the area under the area under the blood plasma concentration curve (AUC0-last) is from about 10734 nM·h to about 16772 nM·h. In some embodiments, the compound, when administered orally to a mouse in an amount of 10 mg per kilogram of the mouse's body weight (mpk), results in a maximum blood plasma concentration (Cmax) in the mouse of from about 1056 to about 1651 nM. In some embodiments, the compound, when administered orally to a mouse in an amount of 10 mg per kilogram of the mouse's body weight (mpk), the area under the area under the blood plasma concentration curve (AUC0-last) is from about 13132 nM·h to about 20519 nM·h. In some embodiments, the compound, when administered orally to a mouse in an amount of 30 mg per kilogram of the mouse's body weight (mpk), results in a maximum blood plasma concentration (Cmax) in the mouse of from about 2744 to about 4288 nM. In some embodiments, the compound, when administered orally to a mouse in an amount of 30 mg per kilogram of the mouse's body weight (mpk), the area under the area under the blood plasma concentration curve (AUC0-last) is from about 44791 nM·h to about 69986 nM·h. In some embodiments, the compound has a structure of Formula (Ib), or a pharmaceutically acceptable salt, polymorph, solvate, enantiomer, stereoisomer, or prodrug thereof. In some embodiments, the compound, when administered orally to a mouse in an amount of 10 mg per kilogram of the mouse's body weight (mpk), results in a maximum blood plasma concentration (Cmax) in the mouse of from about 1338 to about 2091 nM. In some embodiments, the compound, when administered orally to a mouse in an amount of 10 mg per kilogram of the mouse's body weight (mpk), the area under the area under the blood plasma concentration curve (AUC0-last) is from about 5681 nM·h to about 8876 nM·h. In some embodiments, the compound, when administered orally to a mouse in an amount of 30 mg per kilogram of the mouse's body weight (mpk), results in a maximum blood plasma concentration (Cmax) in the mouse of from about 4374 to about 6834 nM. In some embodiments, the compound, when administered orally to a mouse in an amount of 30 mg per kilogram of the mouse's body weight (mpk), the area under the area under the blood plasma concentration curve (AUC0-last) is from about 29637 nM·h to about 46309 nM·h. In some embodiments, the compound has a structure of Formula (Ia), or a pharmaceutically acceptable salt, polymorph, solvate, enantiomer, stereoisomer, or prodrug thereof. In some embodiments, the compound, when administered orally to a dog in an amount of 1.5 mg per kilogram of the dog's body weight (mpk), results in a maximum blood plasma concentration (Cmax) in the dog of from about 6.7 nM to about 11 nM. In some embodiments, the compound, when administered orally to a dog in an amount of 1.5 mg per kilogram of the dog's body weight (mpk), the area under the area under the blood plasma concentration curve (AUC0-last) is from about 37 nM·h to about 59 nM·h. In some embodiments, the compound, when administered orally to a dog in an amount of 4.5 mg per kilogram of the dog's body weight (mpk), results in a maximum blood plasma concentration (Cmax) in the dog of from about 38 nM to about 60 nM. In some embodiments, the compound, when administered orally to a dog in an amount of 4.5 mg per kilogram of the dog's body weight (mpk), the area under the area under the blood plasma concentration curve (AUC0-last) is from about 367 nM·h to about 574 nM·h. In some embodiments, the compound, when administered orally to a mouse in an amount of 10 mg per kilogram of the mouse's body weight (mpk), results in a maximum blood plasma concentration (Cmax) in the mouse of from about 287 to about 449 nM. In some embodiments, the compound, when administered orally to a mouse in an amount of 10 mg per kilogram of the mouse's body weight (mpk), the area under the area under the blood plasma concentration curve (AUC0-last) is from about 1050 nM·h to about 1641 nM·h. In some embodiments, the compound, when administered orally to a mouse in an amount of 30 mg per kilogram of the mouse's body weight (mpk), results in a maximum blood plasma concentration (Cmax) in the mouse of from about 1020 to about 1595 nM. In some embodiments, the compound, when administered orally to a mouse in an amount of 30 mg per kilogram of the mouse's body weight (mpk), the area under the area under the blood plasma concentration curve (AUC0-last) is from about 4515 nM·h to about 7055 nM·h. In some embodiments, the compound has a structure of Formula (IIIb), or a pharmaceutically acceptable salt, polymorph, solvate, enantiomer, stereoisomer, or prodrug thereof. In some embodiments, the compound, when administered orally to a mouse in an amount of 10 mg per kilogram of the mouse's body weight (mpk), results in a maximum blood plasma concentration (Cmax) in the mouse of from about 1337 to about 2090 nM. In some embodiments, the compound, when administered orally to a mouse in an amount of 10 mg per kilogram of the mouse's body weight (mpk), the area under the area under the blood plasma concentration curve (AUC0-last) is from about 1779 nM·h to about 2780 nM·h. In some embodiments, the compound, when administered orally to a mouse in an amount of 30 mg per kilogram of the mouse's body weight (mpk), results in a maximum blood plasma concentration (Cmax) in the mouse of from about 3627 to about 5668 nM. In some embodiments, the compound, when administered orally to a mouse in an amount of 30 mg per kilogram of the mouse's body weight (mpk), the area under the area under the blood plasma concentration curve (AUC0-last) is from about 6084 nM·h to about 9506 nM·h. In some embodiments, the compound has a structure of Formula (IIIa), or a pharmaceutically acceptable salt, polymorph, solvate, enantiomer, stereoisomer, or prodrug thereof. In some embodiments, the compound passes through the blood brain barrier. In some embodiments, the patient has a brain tumor. In some embodiments, the brain tumor is a metastatic brain tumor, a meningioma, a neuroblastoma, a glioblastoma, or an astrocytoma.

Some embodiments disclosed herein provide method for treating cancer, the method comprising administering to a patient in need thereof a composition comprising an effective amount of a compound having a structure of Formula (IIIb):

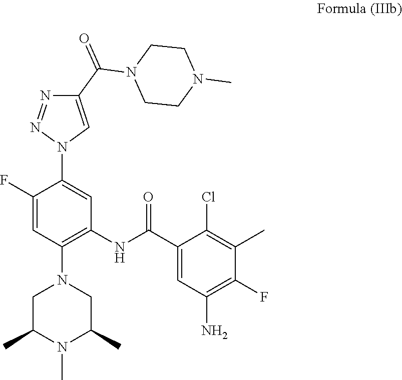 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

In some embodiments of the methods of treating cancer with a compound of Formula (IIIb) (also referred to herein as HBI-2375 or HYBI-084), the compound passes through the blood brain barrier. In some embodiments, the cancer is a solid cancer, hematological cancer or brain cancer. In some embodiments, the effective amount of the compound is about 0.5 to about 20 mg per kg of the patient's body weight (mpk). In some embodiments, the patient is a human and the compound is administered at a dose of about 1 to about 20 mg per kg of the patient's body weight. In some embodiments, the compound is administered to a human at a dose of about 1 to about 20 mg per kg of the patient's body weight, a brain concentration Cₘₐₓ of about 10 to about 310 ng per gram of the patient's estimated brain weight. In some embodiments, the compound, when administered orally to a mouse in an amount of 40 mg per kilogram of the mouse's body weight (mpk), results in a maximum blood plasma concentration (Cmax) in the mouse of from about 770 ng/mL to about 1505 ng/mL in about 30 minutes. In some embodiments, the compound, when administered orally to a mouse in an amount of 40 mg per kilogram of the mouse's body weight (mpk), results in a mean concentration in the mouse brain of from about 58 ng/mL to about 156 ng/mL in about 30 minutes. In some embodiments, the compound, when administered orally to a mouse in an amount of 80 mg per kilogram of the mouse's body weight (mpk), results in a maximum blood plasma concentration (Cmax) in the mouse of from about 2700 ng/mL to about 5200 ng/mL in about 30 minutes. In some embodiments, the compound, when administered orally to a mouse in an amount of 80 mg per kilogram of the mouse's body weight (mpk), results in a mean concentration in the mouse brain of from about 191 ng/mL to about 540 ng/mL in about 30 minutes. In some embodiments, the compound, when administered orally to a mouse in an amount of 40 mg per kilogram of the mouse's body weight (mpk), results in a maximum concentration (Cmax) in the mouse brain of from about 126 ng/mL to about 234 ng/mL in about 4 hours. In some embodiments, the compound, when administered orally to a mouse in an amount of 80 mg per kilogram of the mouse's body weight (mpk), results in a maximum concentration (Cmax) in the mouse brain of from about 360 ng/mL to about 700 ng/mL in about 4 hours. In some embodiments, the compound, when administered orally to a mouse in an amount of 40 mg per kilogram of the mouse's body weight (mpk), the area under the blood plasma concentration curve (AUCₗₐₛₜ) is from about 4600 hr·ng/mL to about 8600 hr·ng/mL. In some embodiments, the compound, when administered orally to a mouse in an amount of 40 mg per kilogram of the mouse's body weight (mpk), the area under the brain concentration curve (AUCₗₐₛₜ) is from about 1400 hr·ng/mL to about 2640 hr·ng/mL. In some embodiments, the compound, when administered orally to a mouse in an amount of 80 mg per kilogram of the mouse's body weight (mpk), the area under the blood plasma concentration curve (AUCₗₐₛₜ) is from about 10900 hr·ng/mL to about 20200 hr·ng/mL. In some embodiments, the compound, when administered orally to a mouse in an amount of 80 mg per kilogram of the mouse's body weight (mpk), the area under the brain concentration curve (AUCₗₐₛₜ) is from about 4090 hr·ng/mL to about 7600 hr·ng/mL. In some embodiments, the compound, when administered orally to a dog in an amount of 1.5 mg per kilogram of the dog's body weight (mpk), results in a maximum blood plasma concentration (Cmax) in the dog of from about 150 to about 235 nM. In some embodiments, the compound, when administered orally to a dog in an amount of 1.5 mg per kilogram of the dog's body weight (mpk), the area under the area under the blood plasma concentration curve (AUC₀₋ₗₐₛₜ) is from about 4225 nM·h to about 6603 nM·h.

Some embodiments described herein provide a compound that inhibits MLL1-WDR5 protein-protein interactions and crosses the blood-brain barrier. In some embodiments, the compound's structure is of Formula (I) or Formula (III).

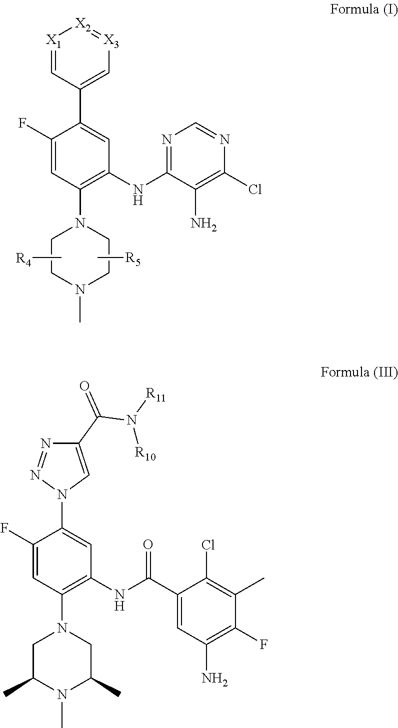

wherein:
X₁ is N or CH;
X₂ is N, CR₂;
X₃ is N or CH;
R₂ is selected from N-morpholino, wherein the morpholino group may be substituted by one or two methyl groups;
each of R₄ and R₅ is hydrogen or alkyl, wherein one or both of R₄ and R₅ may be alkyl; and each of R₁₀ and R₁₁ is C₁-C₄ alkyl or R₁₀ and R₁₁ together form a 4-alkyl piperazinyl group.

In some embodiments, the compound has a structure selected from the group consisting of a Formula (Ia), Formula (Ib), Formula (IIIa) and Formula (IIIb), or a pharmaceutically acceptable salt, polymorph, solvate, enantiomer, stereoisomer, or prodrug thereof:

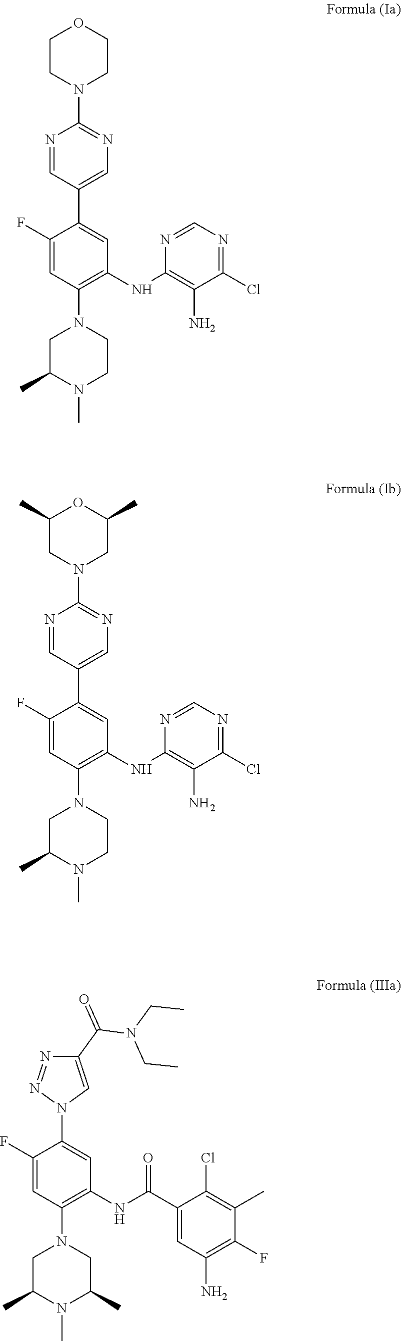 [SMILES: C[C@H]1CN(c2cc(F)c(-c3cnc(N4CCOCC4)nc3)cc2Nc2ncnc(Cl)c2N)CCN1C ; C[C@@H]1CN(c2ncc(-c3cc(Nc4ncnc(Cl)c4N)c(N4CCN(C)[C@@H](C)C4)cc3F)cn2)C[C@H](C)O1 ; CCN(CC)C(=O)c1cn(-c2cc(NC(=O)c3cc(N)c(F)c(C)c3Cl)c(N3C[C@@H](C)N(C)[C@@H](C)C3)cc2F)nn1]

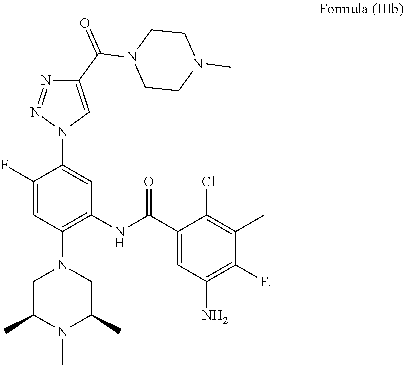 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

In some embodiments, the compound has the structure of Formula (IIIb). In some embodiments, the compound is suitable for use in the treatment of cancer. In some embodiments, the cancer to be treated is a brain cancer. In some embodiments, the cancer to be treated is a primary brain tumor or a secondary brain tumor. In some embodiments, the cancer to be treated is a metastatic tumor. In some embodiments, the cancer to be treated is a glioblastoma, a neuroblastoma, or an astrocytoma.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

FIG. 1A and FIG. 1B: In vitro cell viability of test compounds of this disclosure in MV 411 cells.

FIG. 2: In vitro binding affinity of compounds of this disclosure to WDR5 protein, i.e. MLL1-WDR5 PPI inhibitory activity of representative compounds disclosed herein.

FIG. 3A: Tolerability studies of representative compounds disclosed herein.

FIG. 3B: Tolerability studies of representative compounds disclosed herein.

FIG. 3C: Tolerability studies of representative compounds disclosed herein.

FIG. 3D: Tolerability studies of representative compounds disclosed herein.

FIG. 4A: Efficacy studies of representative compounds disclosed herein in an MV 411 model.

FIG. 4B: Efficacy studies of representative compounds disclosed herein in an MV 411 model.

FIG. 4C: Efficacy studies of representative compounds disclosed herein in an MV 411 model.

FIG. 4D: Efficacy studies of representative compounds disclosed herein in an MV 411 model.

FIG. 5A: Tolerability studies of representative compounds disclosed herein in an MV 411 model.

FIG. 5B: Tolerability studies of representative compounds disclosed herein in an MV 411 model.

FIG. 6A: Pharmacokinetic studies of representative compounds disclosed herein in an in vivo mouse model.

FIG. 6B: Pharmacokinetic studies of representative compounds disclosed herein in an in vivo mouse model.

FIG. 6C: Pharmacokinetic studies of representative compounds disclosed herein in an in vivo mouse model.

FIG. 6D: Pharmacokinetic studies of representative compounds disclosed herein in an in vivo mouse model.

FIG. 7: Pharmacokinetic studies of representative compounds disclosed herein in an MV 411 model.

FIG. 8: Pharmacokinetic studies of representative compounds disclosed herein in a MV 411 model.

FIG. 9A: Pharmacokinetic studies of representative compounds disclosed herein in an in vivo dog model.

FIG. 9B: Pharmacokinetic studies of representative compounds disclosed herein in an in vivo dog model.

FIG. 9C: Pharmacokinetic studies of representative compounds disclosed herein in an in vivo dog model.

FIG. 10A: Pharmacokinetic studies of representative compounds disclosed herein in an in vivo dog model.

FIG. 10B: Pharmacokinetic of representative compounds disclosed herein disclosure in an in vivo dog model.

FIG. 10C: Pharmacokinetic of representative compounds disclosed herein in an in vivo dog model.

FIG. 10D: Pharmacokinetic of representative compounds disclosed herein in an in vivo dog model.

FIG. 11A: Inhibition of HMT 2 &3 activities by representative compounds disclosed herein.

FIG. 11B: Inhibition of HMT 2 &3 activities by representative compounds disclosed herein.

FIG. 11C: Inhibition of HMT 2 &3 activities by representative compounds disclosed herein.

FIG. 12A: Mean concentrations of HBI-2375 in plasma, CSF, and brain tissues of male rats.

FIG. 12B: Mean concentrations of HBI-2375 in plasma, CSF, and brain tissues of female rats.

FIG. 13A: Pharmacokinetic profile (matrix: plasma) of HBI-2375 as measured in an in vivo mouse model.

FIG. 13B: Pharmacokinetic profile (matrix: brain) of HBI-2375 as measured in an in vivo mouse model.

Various embodiments of the disclosure are set forth with particularity in the appended claims. A better understanding of the features and advantages of the present disclosure will be obtained by reference to the following detailed description that sets forth illustrative embodiments below, in which the principles of the disclosure are utilized, and the accompanying drawings.

### DETAILED DESCRIPTION

The phenyl triazole compounds and the aniline compounds as described herein have strong inhibitory activity against MLL1-WDR5 protein-protein interaction. These compounds can reduce the MLL1 catalytic activity of MLL1 at a cellular level, downregulate the expression of Hox and Meis-1 genes, and induce apoptosis of leukemia cells. Additionally, the compounds described herein exhibit good water solubility, blood-brain barrier (BBB) permeability, and pharmaceutical safety and can be used for the treatment of cancers, such as but not limited to leukemia. Compounds having blood-brain barrier permeability, such as those described herein, are useful for treating tumors of the brain, especially those expressing a carcinogenic MLL fusion protein phenotype. Thus, compounds described herein, in addition to having superior bioavailability, additionally may be used to treat brain tumors expressing the MLL fusion protein phenotype, whether they originate in the brain or metastasized from MLL fusion protein expressing primary tumors.

### Compounds

Some embodiments described herein provide a compound that has strong inhibitory activity against MLL1-WDR5 protein-protein interaction. Some such embodiments cross the blood-brain barrier. Some embodiments described herein provide a compound of Formula (I), or a pharmaceutically acceptable salt or solvate thereof:

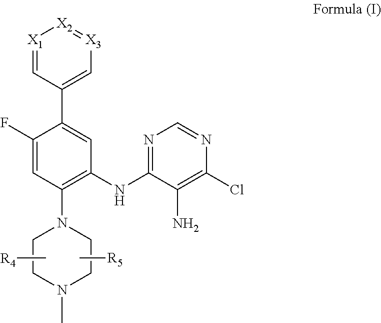

wherein:
   X₁ is N or CH;
   X₂ is N or CR₂;
   X₃ is N or CH;
R₂ is selected from N-morpholino, wherein the morpholino group may be substituted by one or two methyl groups; and
   each of R₄ and R₅ is hydrogen or alkyl, wherein one or both of R₄ and R₅ may be alkyl. In some preferred embodiments, one or both of R₄ and R₅ are alkyl. In some embodiments of the compounds of Formula (I), such compounds cross the blood-brain barrier.

In some embodiments, the compound has the structure of Formula (III), or a pharmaceutically acceptable salt or solvate thereof:

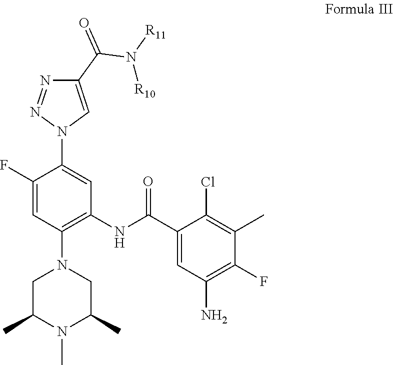

wherein:
   each of R₁₀ and R₁₁ is hydrogen or C₁-C₄ alkyl, or R₁₀ and R₁₁ together form a 4-alkyl piperazinyl group. In some embodiments of the compounds of Formula III, some embodiments described herein cross the blood brain barrier.

In some embodiments, the compound has the structure of Formula (II), or a pharmaceutically acceptable salt or solvate thereof:

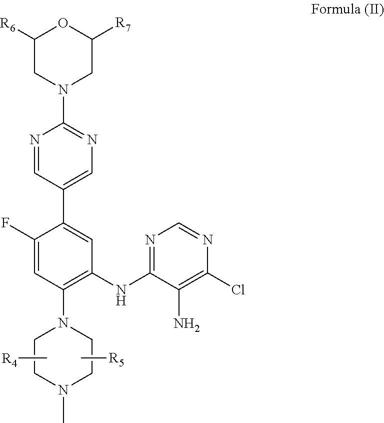

wherein:
each of R₄ and R₅ is hydrogen or methyl, wherein one or both of R₅ and R₆ is methyl, and each of R₆ and R₇ is hydrogen or methyl. In some embodiments of the compounds of Formula II, some embodiments described herein cross the blood brain barrier.

In some embodiments, the compound of Formula (I) or Formula (II) is represented by Formula (Ia) (DDO-2213-20), or a pharmaceutically acceptable salt or solvate thereof:

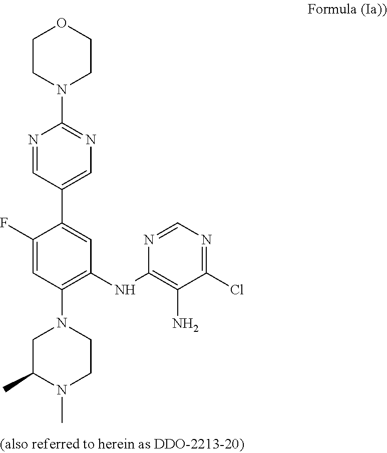 [SMILES: C[C@H]1CN(c2cc(F)c(-c3cnc(N4CCOCC4)nc3)cc2Nc2ncnc(Cl)c2N)CCN1C]

In some embodiments, the compound of Formula (I) is represented by Formula (Ib) (DDO-2213-23), or a pharmaceutically acceptable salt or solvate thereof:

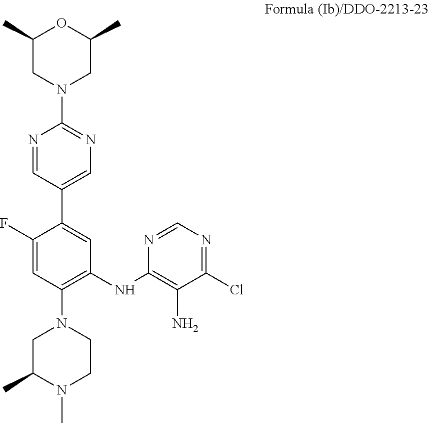 [SMILES: C[C@@H]1CN(c2ncc(-c3cc(Nc4ncnc(Cl)c4N)c(N4CCN(C)[C@@H](C)C4)cc3F)cn2)C[C@H](C)O1]

In some embodiments, the compound of Formula (III) is represented by Formula (IIIa) (HYBI-082), or a pharmaceutically acceptable salt or solvate thereof:

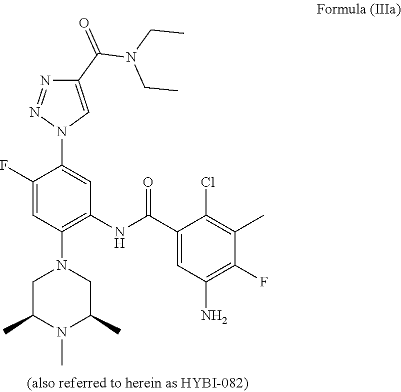 [SMILES: CCN(CC)C(=O)c1cn(-c2cc(NC(=O)c3cc(N)c(F)c(C)c3Cl)c(N3C[C@@H](C)N(C)[C@@H](C)C3)cc2F)nn1]

In some embodiments, the compound of Formula (III) is represented by Formula (IIIb) (HYBI-084), or a pharmaceutically acceptable salt or solvate thereof:

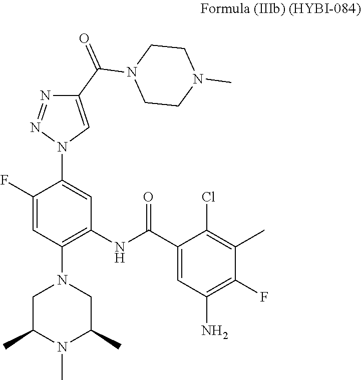 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

Surprisingly, the compound of Formula (IIIb) (HYBI-084) can cross the blood-brain barrier, thereby enabling the compound to inhibit MLL1-WDR5 protein-protein interaction in brain tissues and tissues associated with brain tissues, such as the meninges. Such MLL1-WDR5 protein-protein interaction inhibition activity exposes cancer in the brain and the associated tissues to the anti-cancer activity of the compounds described herein. In some embodiments of the compounds of Formula III, in particular a compound of Formula (IIIb), cross the blood brain barrier, thereby permitting treatment of one or more brain cancers, including primary and secondary brain cancers.

In some embodiments, the compositions described herein comprise a compound of Formula (I), Formula (II), or Formula (III), or a pharmaceutically acceptable salt thereof, or a solvate thereof, together with one or more pharmaceutically acceptable carriers, diluents and excipients. In some embodiments, the compound is a compound of Formula (Ia), Formula (Ib), Formula (IIIa), or Formula (IIIb), or a pharmaceutically acceptable salt thereof, or a solvate thereof.

In some embodiments, compounds described herein include, but are not limited to the compound of Formula (Ia), Formula (Ib), or a pharmaceutically acceptable salt or solvate thereof.

In some embodiments, compounds described herein include, but are not limited to the compound of Formula (IIIa), or Formula (IIIb), or a pharmaceutically acceptable salt or solvate thereof.

In some embodiments, compounds described herein include, but are not limited to the compound of Formula (II), or a pharmaceutically acceptable salt or solvate thereof.

In some embodiments, the present disclosure provides compounds of Formula (I), Formula (II) or formula (III) that cross the BBB. In some embodiments the compounds of Formula (I), Formula (II) or Formula (III) cross the BBB by transmembrane diffusion, saturable transporters, adsorptive endocytosis, or by extracellular pathways. In some embodiments, the compound of Formula (IIIb) crosses the BBB.

In some embodiments, the present disclosure provides a pharmaceutical composition comprising the compound of Formula (I), Formula (II), or Formula (III), or a pharmaceutically acceptable salt thereof, or a solvate thereof, together with one or more pharmaceutically acceptable carriers, diluents and excipients. In some embodiments, the compound of Formula (I), Formula (II), or Formula (III) can cross the blood brain barrier. In some embodiments, after being administered to a subject or a patient, the compound of Formula (I), Formula (II), or Formula (III) can be detected in the cerebrospinal fluid (CSF) of the subject or the patient. In some embodiments, after being administered to a subject or patient, the compound of Formula (I), Formula (II), or Formula (III) can be detected in the brain tissue of the subject or patient. In some embodiments, the compound of Formula (I), Formula (II), or Formula (III) can be detected in the CSF or brain tissue of the subject or patient by one of the methods described herein or other art-recognized methodology. In some embodiments, the compound of Formula (I), Formula (II), or Formula (III), is a compound of Formula (Ia), Formula (Ib), Formula (IIIa), or Formula (IIIb).

Compounds of Formula (I), Formula (II), or Formula (III), is a compound of Formula (Ia), Formula (Ib), Formula (IIIa), or Formula (IIIb) possess superior properties as compared to other members of their class of drug, such as improved bioavailability, superior pharmacokinetics, and ability to cross the blood-brain barrier (BBB). In some embodiments, the compounds possess superior pharmacokinetics (PK) as demonstrated in an animal model, such as mouse, rat, or dog. In some embodiments a compound of the invention, e.g. a compound of Formula (IIIb) (also referred to herein as HBI-2375 or HYBI-084), passes through the blood brain barrier. In some embodiments, when the compound is administered to a human at a dose of about 1 to about 20 mg per kg of the patient's body weight, a brain concentration Cₘₐₓ of about 10 to about 310 ng per gram of the patient's estimated brain weight is achieved. Brain weight may be estimated by measuring or estimating a brain volume, e.g. by one or more standard cranial measurements, e.g. extracranial measurement (e.g., using calipers), computer tomography (CT) scan, magnetic resonance (MR) scan, or similar method of measuring or estimating brain volume and multiplying the estimated or measured brain volume by a standard or population average brain density. In some embodiments, the compound, when administered orally to a mouse in an amount of 40 mg per kilogram of the mouse's body weight (mpk), results in a maximum blood plasma concentration (Cmax) in the mouse of from about 770 ng/mL to about 1505 ng/mL in about 30 minutes. In some embodiments, the compound, when administered orally to a mouse in an amount of 40 mg per kilogram of the mouse's body weight (mpk), results in a mean concentration in the mouse brain of from about 58 ng/mL to about 156 ng/mL in about 30 minutes. In some embodiments, the compound, when administered orally to a mouse in an amount of 80 mg per kilogram of the mouse's body weight (mpk), results in a maximum blood plasma concentration (Cmax) in the mouse of from about 2700 ng/mL to about 5200 ng/mL in about 30 minutes. In some embodiments, the compound, when administered orally to a mouse in an amount of 80 mg per kilogram of the mouse's body weight (mpk), results in a mean concentration in the mouse brain of from about 191 ng/mL to about 540 ng/mL in about 30 minutes. In some embodiments, the compound, when administered orally to a mouse in an amount of 40 mg per kilogram of the mouse's body weight (mpk), results in a maximum concentration (Cmax) in the mouse brain of from about 126 ng/mL to about 234 ng/mL in about 4 hours. In some embodiments, the compound, when administered orally to a mouse in an amount of 80 mg per kilogram of the mouse's body weight (mpk), results in a maximum concentration (Cmax) in the mouse brain of from about 360 ng/mL to about 700 ng/mL in about 4 hours. In some embodiments, the compound, when administered orally to a mouse in an amount of 40 mg per kilogram of the mouse's body weight (mpk), the area under the blood plasma concentration curve (AUCₗₐₛₜ) is from about 4600 hr·ng/mL to about 8600 hr·ng/mL. In some embodiments, the compound, when administered orally to a mouse in an amount of 40 mg per kilogram of the mouse's body weight (mpk), the area under the brain concentration curve (AUCₗₐₛₜ) is from about 1400 hr·ng/mL to about 2640 hr·ng/mL. In some embodiments, the compound, when administered orally to a mouse in an amount of 80 mg per kilogram of the mouse's body weight (mpk), the area under the blood plasma concentration curve (AUCₗₐₛₜ) is from about 10900 hr·ng/mL to about 20200 hr·ng/mL. In some embodiments, the compound, when administered orally to a mouse in an amount of 80 mg per kilogram of the mouse's body weight (mpk), the area under the brain concentration curve (AUCₗₐₛₜ) is from about 4090 hr·ng/mL to about 7600 hr·ng/mL. In some embodiments, the compound, when administered orally to a dog in an amount of 1.5 mg per kilogram of the dog's body weight (mpk), results in a maximum blood plasma concentration (Cmax) in the dog of from about 150 to about 235 nM. In some embodiments, the compound, when administered orally to a dog in an amount of 1.5 mg per kilogram of the dog's body weight (mpk), the area under the area under the blood plasma concentration curve (AUC₀₋ₗₐₛₜ) is from about 4225 nM·h to about 6603 nM·h.

In some embodiments, compounds and pharmaceutical compositions described herein are useful for treating diseases, disorders, or alleviating or eliminating the symptoms of diseases and disorders, such as, for example, cancer. In some embodiments, the cancer includes brain tumor. In some embodiments, the brain tumor may be a primary tumor or a metastasis secondary to another tumor.

In some embodiments, the invention provides the use of a compound of Formula (I), Formula (II), Formula (III), Formula (Ia), Formula (Ib), Formula (IIIa), or Formula (IIIb), or a pharmaceutically acceptable salt or solvate thereof, for the manufacture of a medicament for the treatment of cancer. In some embodiments, a method is provided herein for use of the compounds of Formula (I), Formula (II), Formula (III), Formula (Ia), Formula (Ib), Formula (IIIa), or Formula (IIIb), or a pharmaceutically acceptable salt or solvate thereof, for the manufacture of a medicament for the treatment of cancer. The compounds described herein have superior bioavailability and pharmacokinetic properties compared to other compounds in their class. In some embodiments of the methods of treating cancer with a compound of Formula (IIIb) (also referred to herein as HBI-2375 or HYBI-084), the compound passes through the blood brain barrier. In some embodiments, the cancer is a solid cancer, hematological cancer or brain cancer. In some embodiments, the effective amount of the compound is about 0.5 to about 20 mg per kg of the patient's body weight (mpk). In some embodiments, the patient is a human and the compound is administered at a dose of about 1 to about 20 mg per kg of the patient's body weight. In some embodiments, the compound is administered to a human at a dose of about 1 to about 20 mg per kg of the patient's body weight, a brain concentration Cₘₐₓ of about 10 to about 310 ng per gram of the patient's estimated brain weight. In some embodiments, the compound, when administered orally to a mouse in an amount of 40 mg per kilogram of the mouse's body weight (mpk), results in a maximum blood plasma concentration (Cmax) in the mouse of from about 770 ng/mL to about 1505 ng/mL in about 30 minutes. In some embodiments, the compound, when administered orally to a mouse in an amount of 40 mg per kilogram of the mouse's body weight (mpk), results in a mean concentration in the mouse brain of from about 58 ng/mL to about 156 ng/mL in about 30 minutes. In some embodiments, the compound, when administered orally to a mouse in an amount of 80 mg per kilogram of the mouse's body weight (mpk), results in a maximum blood plasma concentration (Cmax) in the mouse of from about 2700 ng/mL to about 5200 ng/mL in about 30 minutes. In some embodiments, the compound, when administered orally to a mouse in an amount of 80 mg per kilogram of the mouse's body weight (mpk), results in a mean concentration in the mouse brain of from about 191 ng/mL to about 540 ng/mL in about 30 minutes. In some embodiments, the compound, when administered orally to a mouse in an amount of 40 mg per kilogram of the mouse's body weight (mpk), results in a maximum concentration (Cmax) in the mouse brain of from about 126 ng/mL to about 234 ng/mL in about 4 hours. In some embodiments, the compound, when administered orally to a mouse in an amount of 80 mg per kilogram of the mouse's body weight (mpk), results in a maximum concentration (Cmax) in the mouse brain of from about 360 ng/mL to about 700 ng/mL in about 4 hours. In some embodiments, the compound, when administered orally to a mouse in an amount of 40 mg per kilogram of the mouse's body weight (mpk), the area under the blood plasma concentration curve (AUCₗₐₛₜ) is from about 4600 hr·ng/mL to about 8600 hr·ng/mL. In some embodiments, the compound, when administered orally to a mouse in an amount of 40 mg per kilogram of the mouse's body weight (mpk), the area under the brain concentration curve (AUCₗₐₛₜ) is from about 1400 hr·ng/mL to about 2640 hr·ng/mL. In some embodiments, the compound, when administered orally to a mouse in an amount of 80 mg per kilogram of the mouse's body weight (mpk), the area under the blood plasma concentration curve (AUCₗₐₛₜ) is from about 10900 hr·ng/mL to about 20200 hr·ng/mL. In some embodiments, the compound, when administered orally to a mouse in an amount of 80 mg per kilogram of the mouse's body weight (mpk), the area under the brain concentration curve (AUCₗₐₛₜ) is from about 4090 hr·ng/mL to about 7600 hr·ng/mL. In some embodiments, the compound, when administered orally to a dog in an amount of 1.5 mg per kilogram of the dog's body weight (mpk), results in a maximum blood plasma concentration (Cmax) in the dog of from about 150 to about 235 nM. In some embodiments, the compound, when administered orally to a dog in an amount of 1.5 mg per kilogram of the dog's body weight (mpk), the area under the area under the blood plasma concentration curve (AUC₀₋ₗₐₛₜ) is from about 4225 nM·h to about 6603 nM·h.

In some embodiments, the invention provides the use of a compound of Formula (I), Formula (II), Formula (III), Formula (Ia), Formula (Ib), Formula (IIIa), or Formula (IIIb), or a pharmaceutically acceptable salt or solvate thereof, for the manufacture of a medicament for the treatment of a disease mediated by inhibiting MLL1-WDR5 protein-protein interaction.

In some embodiments, the invention provides the use of a compound of Formula (I), Formula (II), Formula (III), Formula (Ia), Formula (Ib), Formula (IIIa), or Formula (IIIb), or a pharmaceutically acceptable salt or solvate thereof, for the manufacture of a medicament for the treatment of MLL gene-fused leukemia.

In some embodiments, the invention provides a method for the treatment of MLL gene-fused leukemia comprising administering a compound of Formula (I), Formula (II), Formula (III), Formula (Ia), Formula (Ib), Formula (IIIa), or Formula (IIIb), or a pharmaceutically acceptable salt or solvate thereof, for the manufacture of a medicament for the treatment of MLL gene-fused leukemia.

In some embodiments, the compounds of Formula (I), Formula (II), Formula (III), Formula (Ia), Formula (Ib), Formula (IIIa), or Formula (IIIb), or pharmaceutically acceptable salts or solvates thereof, are administered in an effective amount from of about 0.1 to about 1000 mg of the compound of Formula (I), Formula (II), Formula (III), Formula (Ia), Formula (Ib), Formula (IIIa), or Formula (IIIb) per kg of the patient's body weight (mpk). Preferably, the effective amount is from of about 0.5 to about 500 mg of the compound per kg of the patient's body weight (mpk). More preferably, the effective amount is from of about 1 to about 100 mg of the compound per kg of the patient's body weight (mpk).

In some embodiments, the compounds of Formula (I), Formula (II), Formula (III), Formula (Ia), Formula (Ib), Formula (IIIa), or Formula (IIIb), or pharmaceutically acceptable salts or solvates thereof, are administered in an effective amount from of about 1.62 to about 6.48 mg of the compound of Formula (I), Formula (II), Formula (III), Formula (Ia), Formula (Ib), Formula (IIIa), or Formula (IIIb) per kg of the patient's body weight (mpk). Preferably, the effective amount is from of about 1.30 to about 8.1 mg per kg of the patient's body weight (mpk). More preferably, the effective amount is from of about 2.59 to about 4.05 mg per kg of the patient's body weight (mpk).

In some embodiments, the compounds of Formula (I), Formula (II), Formula (III), Formula (Ia), Formula (Ib), Formula (IIIa), or Formula (IIIb), or pharmaceutically acceptable salts or solvates thereof, are administered in an effective amount from of about 60 to about 240 mg of the compound of Formula (I), Formula (II), Formula (III), Formula (Ia), Formula (Ib), Formula (IIIa), or Formula (IIIb) per m² of the patient's body surface area. Preferably, the effective amount is from of about 48 to about 300 mg per m² of the patient's body surface area. More preferably, the effective amount is from of about 96 to about 150 mg per m² of the patient's body surface area.

In some embodiments, compounds of the compound of Formula (I), Formula (II), Formula (III), Formula (Ia), Formula (Ib), Formula (IIIa), or Formula (IIIb), or pharmaceutically acceptable salts or solvates thereof, are administered in an effective amount greater than about 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, or any range from one to the other of any two of these amounts, of the compound of Formula (I), Formula (II), Formula (III), Formula (Ia), Formula (Ib), Formula (IIIa), or Formula (IIIb).

In some embodiments, compounds of Formula (I), Formula (II), Formula (III), Formula (Ia), Formula (Ib), Formula (IIIa), or Formula (IIIb), or pharmaceutically acceptable salts or solvates thereof, are administered in an effective amount of at least about 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, or any range from one to the other of any two of these amounts, of the compound of Formula (I), Formula (II), Formula (III), Formula (Ia), Formula (Ib), Formula (IIIa), or Formula (IIIb).

In some embodiments, the compounds of the compound of Formula (I), Formula (II), Formula (III), Formula (Ia), Formula (Ib), Formula (IIIa), or Formula (IIIb), or pharmaceutically acceptable salts or solvates thereof, are administered in an effective amount from of about 1 to about 80 mg of the compound of Formula (I), Formula (II), Formula (III), Formula (Ia), Formula (Ib), Formula (IIIa), or Formula (IIIb) per kg of the patient's body weight (mpk). Preferably, the effective amount is from of about 1 to about 60 mg per kg of the patient's body weight (mpk). More preferably, the effective amount is from of about 1 to about 40 mg per kg of the patient's body weight (mpk).

In some embodiments, the compounds of Formula (I), Formula (II), Formula (III), Formula (Ia), Formula (Ib), Formula (IIIa), or Formula (IIIb), or pharmaceutically acceptable salts or solvates thereof, are administered in an effective amount from of about 1.5 to about 45 mg of the compound of Formula (I), Formula (II), Formula (III), Formula (Ia), Formula (Ib), Formula (IIIa), or Formula (IIIb) per kg of the patient's body weight (mpk). Preferably, the effective amount is from of about 1.5 to about 40 mg per kg of the patient's body weight (mpk). More preferably, the effective amount is from of about 1.5 to about 35 mg per kg of the patient's body weight (mpk). Most preferably, the effective amount is from of about 1.5 to about 30 mg per kg of the patient's body weight (mpk).

In some embodiments, the compounds of Formula (Ia), Formula (Ib), or pharmaceutically acceptable salts or solvates of either thereof, are administered in an effective amount of from about 1.5 to about 45 mg of the compound of Formula (Ia) or Formula (Ib) per kg of the patient's body weight (mpk). Preferably, the effective amount is from of about 1.5 to about 40 mg per kg of the patient's body weight (mpk). More preferably, the effective amount is from of about 1.5 to about 35 mg per kg of the patient's body weight (mpk). Most preferably, the effective amount is from of about 1.5 to about 30 mg per kg of the patient's body weight (mpk).

In some embodiments, the compounds of Formula (IIIa), or Formula (IIIb), or pharmaceutically acceptable salts or solvates thereof, are administered in an effective amount from of about 1.5 to about 45 mg of the compound of Formula (IIIa) or Formula (IIIb) per kg of the patient's body weight (mpk). Preferably, the effective amount is from of about 1.5 to about 40 mg per kg of the patient's body weight (mpk). More preferably, the effective amount is from of about 1.5 to about 35 mg per kg of the patient's body weight (mpk). Most preferably, the effective amount is from of about 1.5 to about 30 mg per kg of the patient's body weight (mpk).

In some embodiments, the effective dose of the compounds of Formula (Ia), Formula (Ib), Formula (IIIa), or Formula (IIIb), or pharmaceutically acceptable salts or solvates thereof, is from about 1 mg to about 6 mg, from about 1 mg to about 5 mg, from about 1.5 mg to about 5 mg, from about 1.5 mg to about 4.5 mg, or from about 15 mg to about 50 mg, from about 15 mg to about 40 mg, from about 10 mg to about 35 mg, or from about 10 mg to about 30 mg, of the compound of Formula (Ia), Formula (Ib), Formula (IIIa), or Formula (IIIb) per kg of the patient's body weight (mpk).

In some embodiments, the compound of Formula (IIIb) or a pharmaceutically acceptable salt or solvate thereof, is administered in an effective amount from of about 0.5 to about 20 mg of compound of Formula (IIIb) per kg of the patient's body weight (mpk). Preferably, the effective amount is from of about 1 to about 10 mg per kg of the patient's body weight (mpk). More preferably, the effective amount is from of about 1.5 to about 7 mg per kg of the patient's body weight (mpk). In some embodiments, the compound, or pharmaceutically acceptable salt of solvate thereof, may be administered at a clinical dose of from about 1.62 mg to 6.48 mg/kg, per day and can be deviated from this range depending on the severity of the condition or the dosage form.

In some embodiments, the peak concentration achieved by the compound of Formula (I), Formula (II), Formula (III), Formula (Ia), Formula (Ib), Formula (IIIa), or Formula (IIIb) is 287 to about 450 nM, preferably about 300 to about 370 nM, more preferably about 330 to about 360 nM or about 1020 to 1600 nM, preferably about 1150 to 1350, more preferably 1250 to 1290 nM.

In some embodiments, the peak concentration achieved by the compounds of this disclosure is 1056 to about 1651 nM, preferably about 1200 to about 1400 nM, more preferably about 1310 to about 1350 nM or about 2744 to 4288 nM, preferably about 3000 to 4000, more preferably 3200 to 3600 nM.

In some embodiments, the peak concentration achieved by the compound of Formula (I), Formula (II), Formula (III), Formula (Ia), Formula (Ib), Formula (IIIa), or Formula (IIIb) is 6.7 to about 11 nM, preferably about 7.5 to about 9.5 nM, more preferably about 8.0 to about 9 nM or about 38 to 60 nM, preferably about 40 to 55 nM, more preferably 45 to 50 nM.

In some embodiments, the peak concentration achieved by t the compound of Formula (I), Formula (II), Formula (III), Formula (Ia), Formula (Ib), Formula (IIIa), or Formula (IIIb) is about 150 to about 235 nM, preferably about 160 to about 220 nM, more preferably about 180 to 195 nM or 356 to about 556 nM, preferably about 400 to about 500 nM, more preferably about 420 to about 460 nM.

In some embodiments, the area under the curve concentration (AUC₀₋ₗₐₛₜ) achieved by the the compound of Formula (I), Formula (II), Formula (III), Formula (Ia), Formula (Ib), Formula (IIIa), or Formula (IIIb) is about 1050 to 1641 nM·h, preferably about 1200 to 1500 nMh, more preferably about 1300 to about 1350 nMh or about 4515 to 7055 nMh, preferably about 5000 to 6500 nMh, more preferably about 5300 to 5800 nMh.

In some embodiments, the area under the curve concentration (AUC₀₋ₗₐₛₜ) achieved by the the compound of Formula (I), Formula (II), Formula (III), Formula (Ia), Formula (Ib), Formula (IIIa), or Formula (IIIb) is about 13132 to 20519 nM·h, preferably about 15500 to 17000 nM·h, more preferably about 16000 to about 16700 nM·h or about 44791 to 69886 nM·h, preferably about 50000 to 60000 nM·h, more preferably about 53000 to 58000 nM·h.

In some embodiments, the area under the curve concentration (AUC₀₋ₗₐₛₜ) achieved by the compound of Formula (I), Formula (II), Formula (III), Formula (Ia), Formula (Ib), Formula (IIIa), or Formula (IIIb) is about 37 to 59 nM·h, preferably about 40 to 55 nM·h, more preferably about 43 to about 48 nM·h; or about 367 to 574 nM·h, preferably about 400 to 500 nM·h, more preferably about 430 to 480 nMh.

In some embodiments, the area under the curve concentration (AUC₀₋ₗₐₛₜ) achieved by the compound of Formula (I), Formula (II), Formula (III), Formula (Ia), Formula (Ib), Formula (IIIa), or Formula (IIIb) is about 4225 to 6603 nM·h, preferably about 4800 to 5800 nM·h, more preferably about 5000 to about 5400 nM·h; or about 10734 to 16772 nM·h, preferably about 12000 to 15000 nMh, more preferably about 13200 to 13800 nM·h.

In some embodiments, the peak concentration in plasma achieved by the compound of Formula (I), Formula (II), Formula (III), Formula (Ia), Formula (Ib), Formula (IIIa), or Formula (IIIb) is 770 ng/mL to about 1505 ng/mL, or about 2750 to about 5200 ng/mL, or about 1700 to about 3300.

In some embodiments, the peak concentration in brain achieved by the compound of Formula (I), Formula (II), Formula (III), Formula (Ia), Formula (Ib), Formula (IIIa), or Formula (IIIb) is 120 ng/mL to about 240 ng/mL, or about 360 to about 700 ng/mL, or about 560 to about 1100 ng/mL.

In some embodiments, the area under the curve concentration (AUCₗₐₛₜ) achieved by the compound of Formula (I), Formula (II), Formula (III), Formula (Ia), Formula (Ib), Formula (IIIa), or Formula (IIIb) in plasma is about 4600 to 8600 hour·ng/mL, or about 10900 to 20300 hour·ng/mL or about 12300 to 23500 hour·ng/mL.

In some embodiments, the area under the curve concentration (AUCₗₐₛₜ) achieved by the compound of Formula (I), Formula (II), Formula (III), Formula (Ia), Formula (Ib), Formula (IIIa), or Formula (IIIb) in brain is about 1400 to 2700 hour·ng/mL, or about 4000 to 7600 hour·ng/mL or about 6999 to 13100 hour·ng/mL.

In some embodiments, the peak concentration in plasma achieved by the compound of Formula (I), Formula (II), Formula (III), Formula (Ia), Formula (Ib), Formula (IIIa), or Formula (IIIb) is 730 ng/mL to about 1110 ng/mL, or about 1240 to about 3100 ng/mL, or about 2050 to about 3000 ng/mL in 1 hour.

In some embodiments, the peak concentration in plasma achieved by the compound of Formula (I), Formula (II), Formula (III), Formula (Ia), Formula (Ib), Formula (IIIa), or Formula (IIIb) is 580 ng/mL to about 900 ng/mL, or about 1360 to about 1740 ng/mL, or about 1040 to about 2070 ng/mL in 4 hours.

In some embodiments, the peak concentration in brain achieved by the compound of Formula (I), Formula (II), Formula (III), Formula (Ia), Formula (Ib), Formula (IIIa), or Formula (IIIb) is 110 ng/mL to about 200 ng/mL, or about 290 to about 500 ng/mL, or about 440 to about 765 ng/mL in 1 hour.

In some embodiments, the peak concentration in brain achieved by the compound Formula (I), Formula (II), Formula (III), Formula (Ia), Formula (Ib), Formula (IIIa), or Formula (IIIb) is 150 ng/mL to about 210 ng/mL, or about 460 to about 600 ng/mL, or about 410 to about 1210 ng/mL in 4 hours.

In some embodiments, the method of treating cancer includes administering the compound of Formula (I), Formula (II), Formula (III), Formula (Ia), Formula (Ib), Formula (IIIa), or Formula (IIIb), or a pharmaceutically acceptable salt or solvate thereof, once per day (Q.D.), twice daily (BID), once every other day (Q.O.D.), every week (Q.W.), two times per week (BIW), three times per week (TIW) or monthly (QM), at any of the dosages provided herein.

In some embodiments, the method of treating cancer includes administering the compound Formula (I), Formula (II), Formula (III), Formula (Ia), Formula (Ib), Formula (IIIa), or Formula (IIIb), or a pharmaceutically acceptable salt or salvate thereof, on a dosing schedule, wherein the dosing schedule includes a first dosing period comprising from about one cycle of administration of the effective dose of the compound, or pharmaceutically acceptable salt or solvate thereof, at a frequency of from about once per week (Q.W.); a first drug holiday; and a second dosing period comprising from about one to about ten cycles of administration of the effective dose of the compound at a frequency of from about once per week (Q.W.) to about three times daily (T.I.D.).

In certain embodiments, the dose of the compound Formula (I), Formula (II), Formula (III), Formula (Ia), Formula (Ib), Formula (IIIa), or Formula (IIIb), or a pharmaceutically acceptable salt or solvate thereof that is administered can be temporarily reduced or temporarily suspended for a certain length of time (i.e., a “drug holiday”), e.g., to mitigate intolerable toxicity.

In some embodiments, the method of treating cancer is such that each treatment cycle is from about seven days or from about twelve days long. In some embodiments, the drug holiday is from about as long as each cycle. In some embodiments, the drug holiday is from about one month, one to six months, or one to twelve months.

In some embodiments, the method of treating cancer includes administering the compound on a dosing schedule, wherein the dosing schedule includes a first dosing period comprising from about one cycle of administration of the effective dose of the compound at a frequency of from about once per week (Q.W.) to about three times per week (T.I.W.), followed by a first drug holiday, which may be followed by one or more additional cycles of treatment and drug holiday.

In some embodiments, the typical daily dose of the compounds of Formula (I), Formula (II), Formula (III), Formula (Ia), Formula (Ib), Formula (IIIa), or Formula (IIIb), or pharmaceutically acceptable salts or solvates thereof, varies within a wide range and will depend on various factors such as the relevant indication, severity of the illness being treated, the route of administration, the age, weight and sex of the patient and the particular compound being used, and can be determined by a physician.

In some embodiments, the compound of Formula (I), Formula (II), Formula (III), Formula (Ia), Formula (Ib), Formula (IIIa), or Formula (IIIb), or a pharmaceutically acceptable salt or solvate thereof, can be administered in a regimen. The regimen can be structured to provide therapeutically effective amounts of the compounds over a predetermined period of time (e.g., an administration time). The regimen can be structured to limit or prevent side-effects or undesired complications of the compounds disclosed herein. Regimens useful for treating cancer can include any number of days of administration which can be repeated as necessary. Administration periods can be broken by a rest period that includes no administration of at least one therapy. For example, a regimen can include administration periods that include 2, 3, 5, 7, 10, 15, 21, 28, or more days. These periods can be repeated. For example, a regimen can include a set number of days as previously described where the regimen is repeated 1, 2, 3, 4, 5, 6, 7, 8, 9, 10, 11, 12, 13, or more times.

Regimens can include a rest period of at least 1, 2, 3, 5, 7, 10, or more days. The rest period can be determined by, for example, monitoring the reaction of the patient to the drug or by measuring the efficacy of the treatment. Rest periods can be applied to all of the therapies administered to the subject such that the subject receives no therapy for a set period of time during the rest period.

Regimens described herein for the treatment of cancer using the compounds of this disclosure or pharmaceutically acceptable salts or solvates thereof, can be continued until disease progression or unacceptable toxicity. In some embodiments, the treatment is continued until disease progression is diminished or reversed (i.e., disease progression form Stage I to Stage II cancer is slowed or reversed). In some embodiments, the treatment is continued until unacceptable toxicity in the patient being treated.

In some embodiments, the compounds described herein are formulated into pharmaceutical compositions.

In some embodiments, the pharmaceutical formulation of the compound Formula (I), Formula (II), Formula (III), Formula (Ia), Formula (Ib), Formula (IIIa), or Formula (IIIb), is administered orally in the form of a tablet. In some embodiments, the pharmaceutical formulation may be in the form of a capsule, gel capsule, caplet, oral suspension, oral or intravenous (IV) solution, etc.

In some embodiments, compounds described herein include, but are not limited to the compounds of Table 1, or a pharmaceutically acceptable salts or solvates thereof.

TABLE 1
Compounds of the disclosure.

| Compound. | |
|---|---|
| No. | Structure |
| 1 (Formula Ia) | 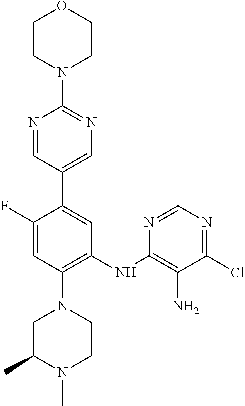 [SMILES: C[C@H]1CN(c2cc(F)c(-c3cnc(N4CCOCC4)nc3)cc2Nc2ncnc(Cl)c2N)CCN1C] |
| 2 (Formula Ib) | 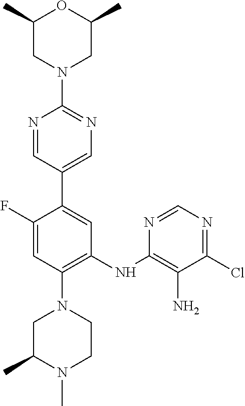 [SMILES: C[C@@H]1CN(c2ncc(-c3cc(Nc4ncnc(Cl)c4N)c(N4CCN(C)[C@@H](C)C4)cc3F)cn2)C[C@H](C)O1] |
| 3 (Formula IIIb) | 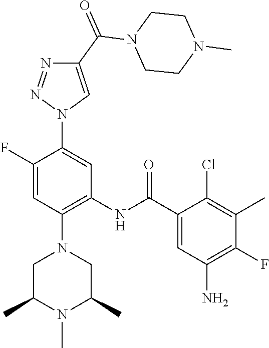 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl] |
| 4 (Formula IIIa) | 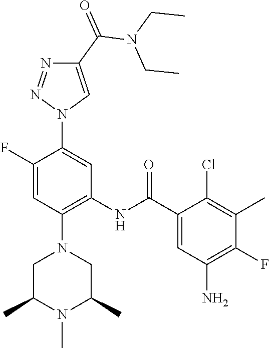 [SMILES: CCN(CC)C(=O)c1cn(-c2cc(NC(=O)c3cc(N)c(F)c(C)c3Cl)c(N3C[C@@H](C)N(C)[C@@H](C)C3)cc2F)nn1] |

In some embodiments, the compound of Formula (I), Formula (II), Formula (III), Formula (Ia), Formula (Ib), Formula (IIIa), or Formula (IIIb) is a compound selected from Table 1, or a pharmaceutically acceptable salt, solvate, polymorph or prodrug thereof.

Any combination of the groups described above for the various variables is contemplated herein. Throughout the specification, groups and substituents thereof are chosen by one skilled in the field to provide stable moieties and compounds.

### Further Forms of Compounds

In some embodiments, a compound disclosed herein possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as stereoisomeric mixtures that are enriched in one of their individual stereoisomers. In some embodiments, the enriched stereoisomer may be prepared by reacting a racemic mixture of the compound with an optically active resolving agent to form a pair of diastereoisomeric compounds/salts, separating the diastereomers and recovering the portions enriched in the optically pure enantiomers. In some embodiments, resolution of enantiomers is carried out using covalent diastereomeric derivatives of the compounds described herein. In other embodiments, diastereomers are separated by separation/resolution techniques based upon differences in solubility. In other embodiments, separation of stereoisomers is performed by chromatography or by the forming of diastereomeric salts and separation is carried out by recrystallization, or chromatography, or any combination thereof. See, e.g., Jean Jacques, Andre Collet, Samuel H. Wilen, “Enantiomers, Racemates and Resolutions,” John Wiley And Sons, Inc., 1981. In some embodiments, stereoisomers may be obtained by stereoselective synthesis.

In some embodiments, compounds described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In some embodiments, prodrugs may be designed to alter the metabolic stability or the transport characteristics of a drug, to mask side effects or toxicity, to improve the flavor of a drug or to alter other characteristics or properties of a drug. By virtue of knowledge of pharmacokinetic, pharmacodynamic processes and drug metabolism in vivo, once a pharmaceutically active compound is known, the design of prodrugs of the compound is possible. (See, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery,” Annual Reports in Medicinal Chemistry, Vol. 41, 395-407, 2006; T. Higuchi and V. Stella, Pro-drugs as Novel Delivery Systems, Vol. 14 of the A.C.S. Symposium Series).

In some embodiments, some of the herein-described compounds may be a prodrug for another derivative or active compound.

In some embodiments, sites on the aromatic ring portion of compounds described herein are susceptible to various metabolic reactions. Therefore, incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In some embodiments, the compounds described herein may be labeled isotopically (e.g., with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine, chlorine, and iodine such as, for example, ²H, ³H, ¹³C, ¹⁴C, ¹⁵N, ¹⁸O, ¹⁷O, ³⁵S, ¹⁸F, ³⁶Cl, and ¹²⁵I. In some embodiments, isotopically-labeled compounds described herein, for example those into which radioactive isotopes such as ³H and ¹⁴C are incorporated, may be useful in drug and/or substrate tissue distribution assays. In some embodiments, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

“Pharmaceutically acceptable” as used herein, refers to a material, such as a carrier or diluent, which does not abrogate the biological activity or properties of the compound, and is relatively nontoxic, i.e., the material can be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound disclosed herein with acids. Pharmaceutically acceptable salts are also obtained by reacting a compound disclosed herein with a base to form a salt.

Compounds of Formula (I), Formula (II), Formula (III), Formula (Ia), Formula (Ib), Formula (IIIa), or Formula (IIIb) can be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g., lithium, sodium, potassium), an alkaline earth ion (e.g., magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Pharmaceutically acceptable salts and solvates may form one or more pharmaceutically acceptable polymorphs, which are also contemplated as being encompassed by the broader class of pharmaceutically acceptable salts or solvates.

### Pharmaceutical Compositions

In some embodiments, the compounds of Formula (I), Formula (II), Formula (III), Formula (Ia), Formula (Ib), Formula (IIIa), or Formula (IIIb) are generally formulated into pharmaceutical compositions for administration to a subject or patient. Thus, described herein are pharmaceutical compositions comprising any one or more of the foregoing compounds, or a pharmaceutically acceptable salt thereof, and optionally one or more additional pharmaceutically acceptable ingredients. Described herein are also methods of making such pharmaceutically acceptable compositions and uses of the compositions in the manufacture of a medicament for the treatment of humans. Pharmaceutical compositions comprising a compound of Formula (I), Formula (II), Formula (III), Formula (Ia), Formula (Tb), Formula (IIIa), or Formula (IIIb), or a pharmaceutically acceptable salt thereof, may be formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound disclosed herein, e.g. a compound of Formula (I), Formula (II), Formula (III), Formula (Ia), Formula (Tb), Formula (IIIa), or Formula (IIIb), or a pharmaceutically acceptable salt thereof, with one or more other chemical components (i.e., ingredients, such as pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally.

In some embodiments, the compounds disclosed herein are administered topically. In such embodiments, the compound disclosed herein is formulated into a variety of topically administrable compositions, such as solutions, suspensions, lotions, gels, pastes, shampoos, scrubs, rubs, smears, medicated sticks, medicated bandages, balms, creams or ointments. In one embodiment, the compounds disclosed herein are administered topically to the skin.

In some embodiments, the compounds disclosed herein are administered by inhalation.

In some embodiments, the compounds disclosed herein are formulated for intranasal administration. Such formulations include nasal sprays, nasal mists, and the like.

In some embodiments, the compounds disclosed herein are formulated as eye drops.

In any of the aforementioned embodiments are further embodiments in which the effective amount of the compound disclosed herein is: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned embodiments are further embodiments comprising single administrations of the effective amount of the compound disclosed herein, including further embodiments in which (i) the compound is administered once; (ii) the compound is administered to a mammal multiple times over the span of one day; (iii) the compound is administered continually; or (iv) the compound is administered continuously.

In any of the aforementioned embodiments are further embodiments comprising multiple administrations of the effective amount of the compound disclosed herein, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound disclosed herein is temporarily suspended or the dose of the compound being administered is temporarily reduced; at the end of the drug holiday, dosing of the compound is resumed. In one embodiment, the length of the drug holiday varies from 2 days to 1 year.

In certain embodiments, the compound disclosed herein is administered in a local rather than systemic manner.

In some embodiments, the compound disclosed herein is administered topically. In some embodiments, the compound disclosed herein is administered systemically.

In some embodiments, the pharmaceutical formulation is in the form of a tablet. In other embodiments, pharmaceutical formulations of the compounds disclosed herein are in the form of a capsule.

In some embodiments, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, the compound disclosed herein is formulated for use as an aerosol, a mist or a powder.

For buccal or sublingual administration, the compositions can take the form of tablets, lozenges, or gels formulated in a conventional manner.

In some embodiments, compounds disclosed herein are prepared as transdermal dosage forms.

In some embodiments, a compound disclosed herein is formulated into a pharmaceutical composition suitable for intramuscular, subcutaneous, or intravenous injection.

In some embodiments, the compound disclosed herein is to be administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

### Methods of Dosing and Treatment Regimens

In some embodiments, the compounds of Formula (I), Formula (II), Formula (III), Formula (Ia), Formula (Ib), Formula (IIIa), or Formula (IIIb) are used in the preparation of medicaments for the treatment of diseases or conditions described herein. In addition, a method for treating any of the diseases or conditions described herein in a subject in need of such treatment, involves administration of pharmaceutical compositions that include at least one compound disclosed herein or a pharmaceutically acceptable salt, active metabolite, prodrug, or solvate thereof, in therapeutically effective amounts to said subject.

In some embodiments of the methods of treating cancer with a compound of Formula (IIIb) (also referred to herein as HBI-2375 or HYBI-084), the compound passes through the blood brain barrier. In some embodiments, the cancer is a solid cancer, hematological cancer or brain cancer. In some embodiments, the effective amount of the compound is about 0.5 to about 20 mg per kg of the patient's body weight (mpk). In some embodiments, the patient is a human and the compound is administered at a dose of about 1 to about 20 mg per kg of the patient's body weight. In some embodiments, the compound is administered to a human at a dose of about 1 to about 20 mg per kg of the patient's body weight, a brain concentration Cₘₐₓ of about 10 to about 310 ng per gram of the patient's estimated brain weight. In some embodiments, the compound, when administered orally to a mouse in an amount of 40 mg per kilogram of the mouse's body weight (mpk), results in a maximum blood plasma concentration (Cmax) in the mouse of from about 770 ng/mL to about 1505 ng/mL in about 30 minutes. In some embodiments, the compound, when administered orally to a mouse in an amount of 40 mg per kilogram of the mouse's body weight (mpk), results in a mean concentration in the mouse brain of from about 58 ng/mL to about 156 ng/mL in about 30 minutes. In some embodiments, the compound, when administered orally to a mouse in an amount of 80 mg per kilogram of the mouse's body weight (mpk), results in a maximum blood plasma concentration (Cmax) in the mouse of from about 2700 ng/mL to about 5200 ng/mL in about 30 minutes. In some embodiments, the compound, when administered orally to a mouse in an amount of 80 mg per kilogram of the mouse's body weight (mpk), results in a mean concentration in the mouse brain of from about 191 ng/mL to about 540 ng/mL in about 30 minutes. In some embodiments, the compound, when administered orally to a mouse in an amount of 40 mg per kilogram of the mouse's body weight (mpk), results in a maximum concentration (Cmax) in the mouse brain of from about 126 ng/mL to about 234 ng/mL in about 4 hours. In some embodiments, the compound, when administered orally to a mouse in an amount of 80 mg per kilogram of the mouse's body weight (mpk), results in a maximum concentration (Cmax) in the mouse brain of from about 360 ng/mL to about 700 ng/mL in about 4 hours. In some embodiments, the compound, when administered orally to a mouse in an amount of 40 mg per kilogram of the mouse's body weight (mpk), the area under the blood plasma concentration curve (AUCₗₐₛₜ) is from about 4600 hr·ng/mL to about 8600 hr·ng/mL. In some embodiments, the compound, when administered orally to a mouse in an amount of 40 mg per kilogram of the mouse's body weight (mpk), the area under the brain concentration curve (AUCₗₐₛₜ) is from about 1400 hr·ng/mL to about 2640 hr·ng/mL. In some embodiments, the compound, when administered orally to a mouse in an amount of 80 mg per kilogram of the mouse's body weight (mpk), the area under the blood plasma concentration curve (AUCₗₐₛₜ) is from about 10900 hr·ng/mL to about 20200 hr·ng/mL. In some embodiments, the compound, when administered orally to a mouse in an amount of 80 mg per kilogram of the mouse's body weight (mpk), the area under the brain concentration curve (AUCₗₐₛₜ) is from about 4090 hr·ng/mL to about 7600 hr·ng/mL. In some embodiments, the compound, when administered orally to a dog in an amount of 1.5 mg per kilogram of the dog's body weight (mpk), results in a maximum blood plasma concentration (Cmax) in the dog of from about 150 to about 235 nM. In some embodiments, the compound, when administered orally to a dog in an amount of 1.5 mg per kilogram of the dog's body weight (mpk), the area under the area under the blood plasma concentration curve (AUC₀₋ₗₐₛₜ) is from about 4225 nM·h to about 6603 nM·h.

In some embodiments, the compositions containing the compound of Formula (I), Formula (II), Formula (III), Formula (Ia), Formula (Tb), Formula (IIIa), or Formula (IIIb) are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds disclosed herein are administered to a patient susceptible to or otherwise at risk of a particular disease, disorder or condition.

In certain embodiments, the dose of drug being administered can be temporarily reduced or temporarily suspended for a certain length of time (i.e., a “drug holiday”).

Doses employed for adult human treatment are typically in the range of 97.2 mg to 388.8 mg of the compound of Formula (I), Formula (II), Formula (III), Formula (Ia), Formula (Ib), Formula (IIIa), or Formula (IIIb) per day or from about 78 mg to about 486 mg per day. In one embodiment, the desired dose is conveniently presented in a single dose or in divided doses.

### Methods of Treatment

Described herein is are methods for the treatment of diseases mediated by MLL1 through inhibiting MLL1-WDR5 protein-protein interaction, wherein the diseases, such as for example MLL gene fusion type leukemia or brain tumor can be treated through inhibition of the enzymatic activity of MLL1. In some embodiments, described herein is a method of treating a disease or condition including administering to a subject in need thereof an effective amount of a compound disclosed herein or a pharmaceutically acceptable salt thereof. Thus, also described herein are compounds and pharmaceutical compositions for use in treating diseases mediated by MLL1 through inhibiting MLL1-WDR5 protein-protein interaction, wherein the diseases, such as for example MLL gene fusion type leukemia or brain tumor can be treated through inhibition of the enzymatic activity of MLL1.

In some embodiments, the disease or condition being treated or to be treated is a cancer. In some embodiments, the cancer is a solid tumor. In some embodiments, the cancer is a brain tumor. In some embodiments, the brain tumor is a primary brain tumor. In some embodiments, the brain tumor is a secondary tumor, e.g., a metastatic brain tumor. In some embodiments, the tumor is a metastatic brain tumor, a meningioma, a neuroblastoma, a glioblastoma, an oligodendroglioma, or an astrocytoma. In some embodiments, the cancer is a glioblastoma or a neuroblastoma. In some embodiments, the cancer is a brain metastasis, e.g., a metastasis derived from one or more of the cancers in the immediately following paragraph.

In some aspects, the cancer being treated or to be treated is one or more of prostate, skin, ovarian cancer; cancers of non-lymphoid parenchymal organs including the heart, placenta, skeletal muscle and lung; breast cancer; cancers of the head and neck including various lymphomas, such as mantle cell lymphoma, Non-Hodgkin B cell lymphoma, PTCL, adenoma, squamous cell carcinoma, laryngeal carcinoma, salivary carcinoma, thymomas and thymic carcinoma; leukemia; cancers of the retina; cancers of the esophagus; multiple myeloma; melanoma; colorectal cancer; lung cancer; cervical cancer; endometrium carcinoma; gallbladder cancer; liver cancer; thyroid follicular cancer; gastric cancer; non-small cell lung carcinoma; glioma; urothelial cancer; bladder cancer; prostate cancer; renal cell cancer; infiltrating ductal carcinoma; and glioblastoma multiform. In some embodiments, the cancer is a hematological cancer or a brain cancer.

In some embodiments, the cancer is a hematological cancer selected from lymphoma, Non-Hodgkin lymphoma (NHL), Hodgkin's Lymphoma, Reed-Stemberg disease, multiple myeloma (MM), acute myelogenous leukemia (AML), chronic myelogenous leukemia (CML), acute lymphocytic leukemia, (ALL), or chronic lymphocytic leukemia (CLL). In certain embodiments, the cancer is Hodgkin's Lymphoma or Reed-Sternberg disease.

### Leukemia

Leukemia is characterized by an abnormal increase of white blood cells in the blood or bone marrow. Among all types of cancers, the morbidity of leukemia is the highest for patients below 35 years old. Over 70% of infant leukemia patients bear a translocation involving chromosome 11, resulting in the fusion of the MLL1 gene with other genes (Nat. Rev. Cancer, 2007, 7(11):823-833). MLL1 translocations are also found in approximately 10% of adult acute myeloid leukemia (AML) patients who were previously treated with topoisomerase II inhibitors for other types of cancers.

MLL1 enzymatic activity is determined by MLL1 and WDR5 protein-protein interaction; MLL1 enzymatic activity affects the methylation level of H3K4. The H3K4 methylation level increases abnormally in MLL fusion type leukemia, and the downstream Hox and Meis-1 gene expression levels are up-regulated abnormally. When MLL1-WDR5 protein-protein interaction is inhibited, e.g., by one of the compounds of Formula (I), Formula (II), Formula (III), Formula (Ia), Formula (Ib), Formula (IIIa), or Formula (IIIb), MLL1 catalytic activity decreases, H3K4 methylation level decreases, Hox and Meis-1 gene expression levels are downregulated, inhibiting leukemia cell proliferation.

In some embodiments, the treated cancer is leukemia. In some embodiments, the leukemia is acute leukemia. In some embodiments, the acute leukemia is acute leukemia with MLL1 gene rearrangement.

### Acute Myeloid Leukemia (AML)

The CEBPA gene is mutated in 9% of patients with acute myeloid leukemia (AML). Selective expression of a short (30-kDa) CCAAT-enhancer binding protein-a (C/EBPa) translational isoform, termed p30, represents the most common type of CEBPA mutation in AML. The molecular mechanisms underlying p30-mediated transformation remain incompletely understood. However, studies have shown that C/EBPa p30, but not the normal p42 isoform, preferentially interacts with WDR5, a key component of SET/MLL (SET-domain/mixed-lineage leukemia) histone-methyltransferase complexes. Accordingly, p30-bound genomic regions are enriched for MLL-dependent H3K4me3 marks. The p30-dependent increase in self-renewal and inhibition of myeloid differentiation required WDR5, as downregulation of the latter inhibited proliferation and restored differentiation in p30-dependent AML models. Small-molecule inhibitors of WDR5-MLL binding selectively inhibited proliferation and induced differentiation in p30-expressing human AML cells revealing the mechanism of p30-dependent transformation and establish the p30 cofactor WDR5 as a therapeutic target in CEBPA-mutant AML (Nat. Chem. Biol. 2015; 11(8):571-8). The inhibition of WDR5-MLL binding by compounds of Formula (I), Formula (II), Formula (III), Formula (Ia), Formula (Ib), Formula (IIIa), or Formula (IIIb), provides the rationale for treatment of AML with one or more of those compounds, or pharmaceutically acceptable salts or solvates thereof. Thus, in some embodiments, the treated leukemia is AML leukemia.

### MYCN-Amplified Neuroblastoma

MYCN gene amplification in neuroblastoma drives a gene expression program that correlates strongly with aggressive disease. Mechanistically, trimethylation of histone H3 lysine 4 (H3K4) at target gene promoters is a prerequisite for the transcriptional program to be enacted. WDR5 is a histone H3K4 presenter that has been found to have an essential role in H3K4 trimethylation. The relationship between WDR5-mediated H3K4 trimethylation and N-Myc transcriptional programs in neuroblastoma cells was investigated. N-Myc upregulated WDR5 expression in neuroblastoma cells. Gene expression analysis revealed that WDR5 target genes included those with MYC-binding elements at promoters such as MDM2. WDR5 has been shown to form a protein complex at the MDM2 promoter with N-Myc, but not p53, leading to histone H3K4 trimethylation and activation of MDM2 transcription (Cancer Res 2015; 75(23); 5143-54). RNAi-mediated attenuation of WDR5 upregulated expression of wild-type but not mutant p53, an effect associated with growth inhibition and apoptosis. Similarly, a small-molecule antagonist of WDR5 reduced N-Myc/WDR5 complex formation, N-Myc target gene expression, and cell growth in neuroblastoma cells. In MYCN-transgenic mice, WDR5 was overexpressed in precancerous ganglion and neuroblastoma cells compared with normal ganglion cells. Clinically, elevated levels of WDR5 in neuroblastoma specimens have an independent predictor of poor overall survival. WDR5 has been identified as a relevant cofactor for N-Myc-regulated transcriptional activation and tumorogenesis and as a novel therapeutic target for MYCN-amplified neuroblastomas (Cancer Res. 2015; 75(23); 5143-54, Mol. Cell. 2015; 58(3):440-52). The inhibition of WDR5-MLL binding by compounds of Formula (I), Formula (II), Formula (III), Formula (Ia), Formula (Ib), Formula (IIIa), or Formula (IIIb), provides the rationale for treatment of MYCN-amplified neuroblastoma with one or more of those compounds, or pharmaceutically acceptable salts or solvates thereof. Thus, in some embodiments, the treated cancer is a solid tumor, such as a brain cancer, e.g., a neuroblastoma, which may be a MYCN-amplified neuroblastoma.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

The terms below, as used herein, have the following meanings, unless indicated otherwise:

“Oxo” refers to the ═O substituent.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. Unless stated otherwise specifically in the specification, an alkyl group may be optionally substituted as described below.

“Alkylene” refers to a straight or branched divalent hydrocarbon chain linking the rest of the molecule to a radical group. In some embodiments, the alkylene is —CH₂—, —CH₂CH₂—, —CH₂CH₂CH₂—, or —CH₂CH₂CH₂CH₂—.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

“Heteroalkyl” refers to an alkyl radical as described above where one or more carbon atoms of the alkyl is replaced with a O, N (i.e., NH, N-alkyl) or S atom. “Heteroalkylene” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized π-electron system containing 4n+2π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group.

“Cycloalkyl” refers to a monocyclic or polycyclic non-aromatic radical, wherein each of the atoms forming the ring (i.e., skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. In some embodiments, a cycloalkyl is a C₃-C₆ cycloalkyl. In some embodiments, the cycloalkyl is monocyclic, bicyclic or polycyclic. In some embodiments, the cycloalkyl is monocyclic. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the cycloalkyl is bicyclic. In some embodiments, the cycloalkyl is polycyclic. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, bicyclic ring (which may include a fused bicyclic heterocycloalkyl (when fused with an aryl or a heteroaryl ring, the heterocycloalkyl is bonded through a non-aromatic ring atom), bridged heterocycloalkyl or spiro heterocycloalkyl), or polycyclic. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆ alkylalkyne, halogen, acyl, acyloxy, —CO₂H, —CO₂ alkyl, nitro, and amino, including mono- and di-substituted amino groups (e.g., —NH₂, —NHR, —NR₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂ alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutical combination” as used herein, means a product that results from the mixing or combining of more than one active ingredient and includes both fixed and non-fixed combinations of the active ingredients. The term “fixed combination” means that the active ingredients, e.g., a compound of Formula (I), Formula (II), Formula (III), Formula (Ia), Formula (Ib), Formula (IIIa), or Formula (IIIb) and a co-agent, are both administered to a patient simultaneously in the form of a single entity or dosage. The term “non-fixed combination” means that the active ingredients, e.g., a compound of Formula (I), Formula (II), Formula (III), Formula (Ia), Formula (Tb), Formula (IIIa), or Formula (IIIb) and a co-agent, are administered to a patient as separate entities, either simultaneously, concurrently or sequentially with no specific intervening time limits, wherein such administration provides effective levels of the two gents in the body of the patient. The latter also applies to cocktail therapy, e.g., the administration of three or more active ingredients. Thus, combination therapy is distinct from separate therapy, which would comprise cessation of administration of one agent followed by a suitable wash-out period before commencing administration of the other agent.

The term “subject” or “patient” encompasses mammals. Examples of mammals include, but are not limited to, humans. In one embodiment, the mammal is a human. Unless otherwise specified, the terms “subject” and “patient” are intended to be synonymous, whether they are treated as part of a pre-clinical trial or clinical trial, or in a post-approval clinical setting.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Methods of Synthesis

In some embodiments, the syntheses of compounds described herein are accomplished using using the methods described herein, means described in the chemical literature (e.g., WO 2019205687 A1, WO 2020172932 A1, U.S. Pat. No. 11,479,545 B2, and US 2022/0152027 A1, all of which are incorporated herein by reference in their entirety), or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

It is understood that other analogous procedures and reagents could be used, and that these Schemes are only meant as non-limiting examples.

### EXAMPLES

### Pharmaceutical Compositions

### Example A-1: Parenteral Pharmaceutical Composition

To prepare a parenteral pharmaceutical composition suitable for administration by injection (subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl cellulose, propylene glycol, isopropyl myristate and purified alcohol USP. The resulting gel mixture is then incorporated into containers, such as tubes, which are suitable for topical administration.

### SYNTHESIS OF COMPOUNDS

The compounds of Table 2 may be synthesized as described in the following paragraphs.

TABLE 2
Compound Names and their Formulae

| Compound Number/Name | Formula |
|---|---|
| DDO-2213_023 | Formula (IIb) |
| HYBI-084/HBI-2375 | Formula (IIIb) |
| HYBI-082 | Formula (IIIa) |
| DDO-2213_020 | Formula (IIa) |

### HYBI-084 (Formula (IIIb)): Synthesis of 5-amino-2-chloro-4-fluoro-N-(4-fluoro-5-(4-(4-methylpiperazine-1-carbonyl)-1H-1,2,3-triazol-1-yl)-2-((3S,5R)-3,4,5-trimethylpiperazin-1-yl)phenyl)-3-methylbenzamide

To a solution of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (100 mg, 187.28 μmol, 1 eq.) and 1-methylpiperazine (38 mg, 374.56 μmol, 41.55 μL, 2 eq.) in DMF (5 mL) was added HATU (107 mg, 280.92 μmol, 1.5 eq) and DIEA (73 mg, 561.84 μmol, 97.86 μL, 3 eq). The mixture was stirred at 25° C. for 12 hr. The reaction mixture was concentrated directly. The residue was purified by prep-HPLC (Column: Phenomenex Gemini-NX C18 75*30 mm*3 μm; Mobile Phase A: purified water (0.04% NH3H2O+10 mM NH4HCO3); Mobile Phase B: acetonitrile; Gradient: 26-66% B in 11 min.). HYBI-084 (13.2 mg, 20.57 μmol, 10.98% yield, 96.02% purity) was obtained as a yellow solid.

1H NMR: (DMSO-d6, 400 MHz) δ H=9.62 (s, 1H), 8.96 (d, J=1.2 Hz, 1H), 8.22 (d, J=8.0 Hz, 1H), 7.33 (d, J=12.4 Hz, 1H), 6.81 (d, J=9.2 Hz, 1H), 5.49 (s, 2H), 3.98 (s, 2H), 3.66 (s, 2H), 3.08 (d, J=10.8 Hz, 2H), 2.58-2.52 (m, 2H), 2.38 (t, J=4.8 Hz, 4H), 2.35-2.29 (m, 2H), 2.26-2.17 (m, 9H), 1.02 (d, J=6.0 Hz, 6H).

HPLC: Rt=3.439 min in 8 min chromatography, XBridge Shield RP18, 5 μm, 2.1*50 mm, purity 96.02%.

LCMS: Rt=2.636 min in 4 min chromatography, XBridge Shield RP18, 5 μm, 2.1*50 mm, purity 97.89%, MS ESI calcd. for 615.26 [M+H]+ 616.26, found 616.3.

### HYBI-082: Synthesis of 1-(5-(5-amino-2-chloro-4-fluoro-3-methylbenzamido)-2-fluoro-4-((3S,5R)-3,4,5-trimethylpiperazin-1-yl)phenyl)-N,N-diethyl-1H-1,2,3-triazole-4-carboxamide

To a solution of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (100 mg, 187.28 mol, 1 eq.) and N-ethylethanamine (27 mg, 374.56 μmol, 38.58 μL, 2 eq.) in DMF (5 mL) was added HATU (107 mg, 280.92 μmol, 1.5 eq) and DIEA (73 mg, 561.84 μmol, 97.86 μL, 3 eq.). The mixture was stirred at 25° C. for 12 hr. The reaction mixture was concentrated directly. The residue was purified by prep-HPLC (Column: Phenomenex Gemini-NX C18 75*30 mm*3 μm; Mobile Phase A: purified water (0.04% NH3H2O+10 mM NH4HCO3); Mobile Phase B: acetonitrile; Gradient: 29-69% B in 11 min). HYBI-082 (10 mg, 16.82 μmol, 8.98% yield, 99.101% purity) was obtained as a red solid.

1H NMR: (DMSO-d6, 400 MHz) δ H=9.61 (s, 1H), 8.93 (d, J=1.6 Hz, 1H), 8.23 (d, J=8.0 Hz, 1H), 7.33 (d, J=12.4 Hz, 1H), 6.81 (d, J=9.2 Hz, 1H), 5.49 (s, 2H), 3.75 (q, J=6.8 Hz, 2H), 3.47 (q, J=7.2 Hz, 2H), 3.08 (d, J=10.8 Hz, 2H), 2.58-2.51 (m, 2H), 2.39-2.28 (m, 2H), 2.24 (d, J=2.4 Hz, 3H), 2.19 (s, 3H), 1.24 (t, J=6.8 Hz, 3H), 1.16 (t, J=7.2 Hz, 3H), 1.02 (d, J=6.0 Hz, 6H).

HPLC: Rt=2.973 min in 8 min chromatography, XBridge Shield RP18, 5 μm, 2.1*50 mm, purity 99.10%.

LCMS: Rt=2.368 min in 4 min chromatography, XBridge Shield RP18, 5 μm, 2.1*50 mm, purity 98.77%, MS ESI calcd. for 588.25 [M+H]+ 589.25, found 589.3.

### DDO-2213_023: (S)-6-chloro-N4-(2-(3,4-dimethylpiperazin-1-yl)-4-fluoro-5-(2-morpholinopyrimidin-5-yl)phenyl)pyrimidine-4,5-diamine

### Step 1: (S)-6-chloro-N4-(2-(3,4-dimethylpiperazin-1-yl)-4-fluoro-5-(2-morpholinopyrimidin-5-yl)phenyl)pyrimidine-4,5-diamine (DDO-2213_020)

A mixture of DDO-2213_001_5 (150 mg, 349.07 μmol, 1 eq.), 4-[5-(4,4,5,5-tetramethyl-1,3,2-dioxaborolan-2-yl)pyrimidin-2-yl]morpholine (122 mg, 418.88 μmol, 1.2 eq.), Pd(dppf)Cl2·CH2Cl2 (28 mg, 34.91 μmol, 0.1 eq.) and Cs2CO₃ (227 mg, 698.13 μmol, 2 eq.) in dioxane (4 mL) and H2O (0.8 mL) was degassed and purged with N2 for 3 times, and then the mixture was stirred at 100° C. for 12 hr under N2 atmosphere. The reaction mixture was diluted with DCM (100 mL), washed with brine (50 mL). The organic layer was dried over Na2SO4, filtered and concentrated under reduced pressure to give a residue. The residue was purified by prep-HPLC (Column: Phenomenex Gemini-NX C18 75*30 mm*3 μm; Mobile Phase A: purified water (0.05% NH3H₂O+10 mM NH4HCO3); Mobile Phase B: acetonitrile; Gradient: 30-60% B in 11 min.). DDO-2213_020 (69.3 mg, 133.60 μmol, 38.27% yield, 99.09% purity) was obtained as a white solid.

1H NMR (DMSO-d6, 400 MHz) δ H=8.55 (s, 2H), 8.11 (s, 1H), 7.90 (d, J=8.8 Hz, 1H), 7.81 (s, 1H), 7.11-7.01 (m, 1H), 5.35 (s, 2H), 3.77-3.63 (m, 8H), 2.99-2.86 (m, 2H), 2.80-2.69 (m, 2H), 2.47-2.39 (m, 1H), 2.31-2.23 (m, 1H), 2.18 (s, 4H), 0.95 (d, J=6.0 Hz, 3H).

HPLC Rt=3.487 min in 8 min chromatography, XBridge Shield RP18, 5 μm, 2.1*50 mm, purity 99.09%.

LCMS Rt=2.674 min in 4 min chromatography, XBridge Shield RP18, 5 μm, 2.1*50 mm, purity 99.49%, MS ESI calcd. for 513.22 [M+H]+ 514.22, found 514.3.

### DDO-2213_023: chloro-N4-(5-(2-((2S,6R)-2,6-dimethylmorpholino)pyrimidin-5-yl)-2-((S)-3,4-dimethylpiperazin-1-yl)-4-fluorophenyl)pyrimidine-4,5-diamine

### Step 1: (2S,6R)-4-(5-bromopyrimidin-2-yl)-2,6-dimethylmorpholine (Compound 2A)

To a solution of compound 1A (1 g, 5.17 mmol, 1 eq.) and (2S,6R)-2,6-dimethylmorpholine (595 mg, 5.17 mmol, 1 eq.) in MeCN (15 mL) was added DIEA (1.34 g, 10.34 mmol, 1.80 mL, 2 eq.). The mixture was stirred at 25° C. for 12 hr. The reaction mixture was diluted with water (20 mL), extracted with DCM (100 mL) and washed with brine (20 mL). The organic layer was dried over Na2SO4, filtered and concentrated under reduced pressure to give a residue. The residue was purified by flash silica gel chromatography (ISCO®; 12 g SepaFlash® Silica Flash Column, Eluent of 0-2% Methanol/Dichloromethane @ 40 mL/min). Compound 2A (1.4 g, 5.02 mmol, 97.02% yield) was obtained as a white solid.

1H NMR (DMSO-d6, 400 MHz) δ H=8.46 (s, 2H), 4.41 (dd, J=1.6, 13.2 Hz, 2H), 3.54 (dd, J=2.4, 4.4 Hz, 2H), 2.58-2.51 (m, 2H), 1.14 (d, J=6.4 Hz, 6H).

### Step 2: (2S,6R)-2,6-dimethyl-4-(5-(4,4,5,5-tetramethyl-1,3,2-dioxaborolan-2-yl)pyrimidin-2-yl)morpholine (Compound 3A)

To a mixture of compound 2A (1 g, 3.67 mmol, 1 eq.) in dioxane (17 mL) was added AcOK (721 mg, 7.35 mmol, 2 eq.), Pin2B2 (1.12 g, 4.41 mmol, 1.2 eq.) and Pd(dppf)Cl2 (269 mg, 367.46 μmol, 0.1 eq.) under N2. The reaction mixture was stirred at 100° C. for 2 hr. The mixture was diluted with H2O (20 mL) and extracted with DCM (100 mL*2). The combined organic phase was washed with brine (100 mL), dried over anhydrous Na2SO4, filtered and concentrated in vacuum. The residue was purified by flash silica gel chromatography (ISCO®; 12 g SepaFlash® Silica Flash Column, Eluent of 0-5% Ethyl acetate/DCM @ 30 m/min). Compound 3A (1.1 g, 3.10 mmol, 84.40% yield) was obtained as a brown oil.

1H NMR (DMSO-d6, 400 MHz) δ H=8.51-8.48 (m, 1H), 8.49 (s, 1H), 4.61-4.54 (m, 2H), 3.56-3.49 (m, 2H), 3.56-3.49 (m, 1H), 2.60-2.53 (m, 2H), 1.16 (s, 12H), 1.07 (s, 6H).

### Step 3: 6-chloro-N4-(5-(2-((2S,6R)-2,6-dimethylmorpholino)pyrimidin-5-yl)-2-((S)-3,4-dimethylpiperazin-1-yl)-4-fluorophenyl)pyrimidine-4,5-diamine (DDO-2213_023)

A mixture of compound 5 (70 mg, 162.90 μmol, 1 eq.), compound 3A (63 mg, 195.48 mol, 1.2 eq.), Pd(dppf)Cl2·CH2Cl2 (13 mg, 16.29 μmol, 0.1 eq.) and Cs2CO₃ (106 mg, 325.80 mol, 2 eq.) in dioxane (4 mL) and H2O (0.8 mL) was degassed and purged with N2 for 3 times, and then the mixture was stirred at 100° C. for 12 hr under N2 atmosphere. The reaction mixture was diluted with H2O (10 mL), extracted with DCM (50 mL) and washed with brine (20 mL). The organic layer was dried over Na2SO4, filtered and concentrated under reduced pressure to give a residue. The residue was purified by prep-HPLC (Column: Phenomenex Gemini-NX C18 75*30 mm*3 μm; Mobile Phase A: purified water (0.04% NH3H₂O+10 mM NH4HCO3); Mobile Phase B: acetonitrile; Gradient: 40-70% B in 11 min.). Compound DDO-2213_023 (15.1 mg, 27.77 μmol, 17.05% yield, 99.7% purity) was obtained as a white solid.

1H NMR (DMSO-d6, 400 MHz) δ H=8.53 (s, 2H), 8.10 (s, 1H), 7.88 (d, J=8.8 Hz, 1H), 7.81 (s, 1H), 7.06 (d, J=12.4 Hz, 1H), 5.35 (s, 2H), 4.60-4.48 (m, 2H), 3.63-3.50 (m, 2H), 2.98-2.86 (m, 2H), 2.81-2.70 (m, 2H), 2.62-2.52 (m, 2H), 2.43 (s, 1H), 2.31-2.22 (m, 1H), 2.18 (s, 3H), 2.16-2.11 (m, 1H), 1.16 (d, J=6.0 Hz, 6H), 0.95 (d, J=6.0 Hz, 3H).

HPLC Rt=2.997 min in 8 min chromatography, X Bridge Shield RP18, 5 μm, 2.1*50 mm, purity 99.78%.

LCMS Rt=2.406 min in 4 min chromatography, X Bridge Shield RP18, 5 μm, 2.1*50 mm, purity 99.78%, MS ESI calcd. for 541.25 [M+H]+ 542.25, found 542.4.

### Biological Examples

Cell lines and maintenance: All cell lines (MV 411) used for in vitro and in vivo studies were supplied as part of Wuxi AppTech cell bank. Cell lines were maintained in a medium supplemented with 10% heat inactivated fetal bovine serum at 37° C., in an atmosphere of 5% CO₂, in air. The tumor cells were routinely subcultured twice weekly. The cells growing in an exponential growth phase were used for the in vitro cell potency and in vivo efficacy studies.

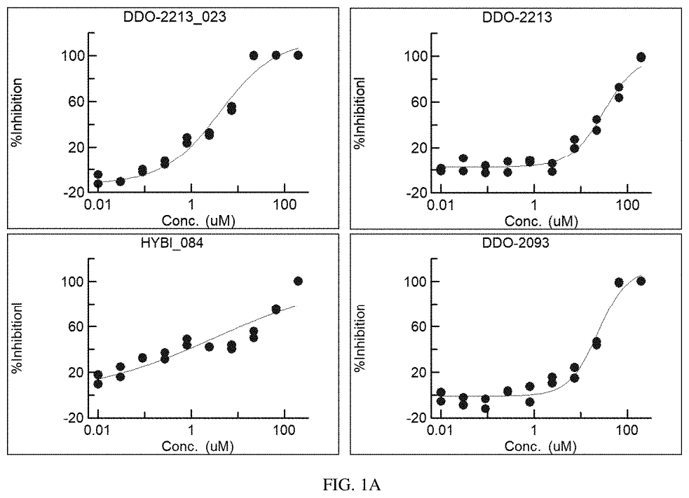

EXAMPLE 1: Cell Viability Assay: For cell cytotoxicity assays, 2000 cells/well were plated in 384-well plates. Twenty four hours after plating, cells were treated with inhibitors at the indicated compound concentrations. On Day 3 (72 hours), cells were lysed with CellTiter-Glo® Luminescent Cell Viability Assay reagent (Promega) and luminescence was read using the EnVision Multi-plate reader. To calculate IC₅₀, a dose-response curve is generated using a nonlinear regression model with a sigmoidal dose response. The formula of surviving rate is shown below. IC₅₀ values were automatically generated by GraphPad Prism 8.0. See FIG. 1A and FIG. 1B and Tables 3 and 4.

$$The surviving rate ( \% ) = ( \frac{LumTest article - LumMedium control}{LumNone treated - LumMedium control} ) \times 100$$

TABLE 3
Selective binding to WDR5 shown by representative
compounds disclosed herein

| | | HBI-2375 (HBI_084) |
|---|---|---|
| No. | Target | IC₅₀ (nM) |
| 1 | ATX | >1000 |
| 2 | BRD2-BD1 | >1000 |
| 3 | BRD2-BD2 | >1000 |
| 4 | BRD3-BD1 | >1000 |
| 5 | BRD4-BD1 | >1000 |
| 6 | BRD4-BD2 | >1000 |
| 7 | BRDT-BD1 | >1000 |
| 8 | CD39 | >1000 |
| 9 | CD73 | >1000 |
| 10 | GLS1 | >1000 |
| 11 | HDAC1 | >1000 |
| 12 | HDAC2 | >1000 |
| 13 | HDAC3 | >1000 |
| 14 | HDAC6 | >1000 |
| 15 | HDAC8 | >1000 |
| 16 | KRAS G12C | >1000 |
| 17 | KRAS G12D | >1000 |
| 18 | KRAS G12V | >1000 |
| 19 | KRAS WT | >1000 |
| 20 | LSD1 | >1000 |
| 21 | MALTI | >1000 |
| 22 | PD-1/PD-L1 | >1000 |
| 23 | POLQ ATPase | >1000 |
| 24 | POLQPolymerase | >1000 |
| 25 | PRMT5 | >1000 |
| 26 | PTP1B | >1000 |
| 27 | SHP2 D61G | >1000 |
| 28 | SHP2 E76K | >1000 |
| 29 | SHP2 WT | >1000 |
| 30 | TCPTP | >1000 |
| 31 | TEAD1 | >1000 |
| 32 | TEAD2 | >1000 |
| 33 | TEAD3 | >1000 |
| 34 | TEAD4 | >1000 |
| 35 | WDR5 | 0.683 |
| 36 | WIP 1 | >1000 |

TABLE 4
Anti-proliferative activity against leukemia
cells of some of the compounds.

| Compound No | IC₅₀ μM |
|---|---|
| DDO-2213_023 | 4.06 |
| HYBI-084 | 3.17 |
| HYBI-082 | 7.68 |
| DDO-2213_020 | 5.81 |

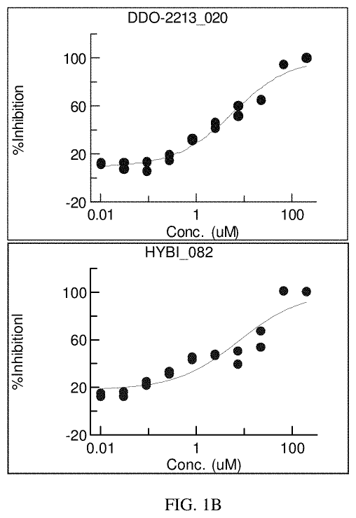

WDR5 TR-FRET Assay Procedure: Stock compounds were transferred to the assay plate by Echo Liquid Handler. Reactions were performed in the assay buffer (1×PBS, 300 mM NaCl, 0.5 mM TCEP, 0.1% CHAPS) containing 5 nM WDR5 protein, 10 nM peptide (Ac-ARTEVHLRKS-[Ahx-Ahx][C]-Alexa Fluor 488-NH2) and 0.25 nM Tb-anti His antibody (Tb-Ab) in 384-well white plate (PerkinElmer) with a final volume of 20 μl. Compounds were incubated with WDR5 protein for 30 min at room temperature. Plates were covered, protected from light and incubated for 60 min. at room temperature after adding the peptide and Tb-Ab. EnVision Multimode Plate Reader (PerkinElmer) was used for the TR-FRET assay with excitation wavelength at 340 nm and emission wavelength at 495 and 520 nm. The ratio of the 520/495 nm wavelengths were used to assess the degree of the FRET signal. IC₅₀ was calculated by fitting the inhibition data using XLfit software to sigmoidal dose-response model. See FIG. 2.

TABLE 5
MLL1-WDR5 PPI inhibitory activity of
representative compounds disclosed herein.

| Compound No | IC₅₀ nM |
|---|---|
| DDO-2213_023 | 3.56 |
| HYBI-084 | 4.48 |
| HYBI-082 | 5.32 |
| DDO-2213_020 | 7.66 |

Compound formulation stability assay: Test articles were formulated in 10% DMSO/10% solutol HS 15/80% water and stored at 4° C. Formulated compounds were analyzed by a HPLC-UV method for actual concentrations and % remaining on Day 1, 4 and 7 after formulation to determine the stability of the formulated test article. The HPLC system was equipped with Shimadzu LC-20 high-performance liquid chromatograph, SIL-20AC auto sampler, LC-20AB pump and SPD-M20A detector. A Waters XBridge C18 (4.6*100 mm) column was applied for the separation. Formulated compounds were analyzed on Day 1, 4 and 7 after formulation to determine the stability of the formulated test article. The results are summarized in Table 6.

TABLE 6
Formulation stability studies of representative compounds disclosed herein.

| Compound | DDO-2213-023 | HYBI-084 | DDO-2213-020 | HYBI-082 |
|---|---|---|---|---|
| Concentration | (10 mg/mL) | (10 mg/mL) | (10 mg/mL) | (10 mg/mL) |
| Vehicle | 10% DMSO/10% solutol HS 15/80% water | | | |
| Formulation | | | | |
| Procedure | stirred 10 | stirred 5 | stirred 10 | stirred 5 |
| | minutes | minutes | minutes | minutes |
| Formulation Storage | 2-8° C. | 2-8° C. | 2-8° C. | 2-8° C. |
| Condition | | | | |
| Formulation | clear solution | clear solution | clear solution | clear |
| Appearance (Day 0) | | | | solution |
| Accuracy % (Day 0) | 89.9 | 92.8 | 89.8 | 96.3 |
| Remaining % (Day 0) | 100 | 100 | 100 | 100 |
| Formulation | clear solution | clear solution | clear solution | clear |
| Appearance (Day 1) | | | | solution |
| Accuracy % (Day 1) | 81.3 | 93.4 | 91.6 | 96.0 |
| Remaining % (Day 1) | 90.4 | 101 | 102 | 100 |
| Formulation | clear solution | clear solution | clear solution | clear |
| Appearance (Day 4) | | | | solution |
| Accuracy % (Day 4) | 92.2 | 92.9 | 92.0 | 99.3 |
| Remaining % (Day 4) | 100 | 100 | 100 | 103 |
| Formulation | clear solution | clear solution | clear solution | clear |
| Appearance (Day 7) | | | | solution |
| Accuracy % (Day 7) | 91.5 | 91.5 | 95.5 | 98.6 |
| Remaining % (Day 7) | 99.1 | 98.7 | 104 | 102 |

EXAMPLE 2: Animal models and in vivo treatments: Procedures involving the care and use of animals in this study were reviewed and approved by the Institutional Animal Care and Use Committee (IACUC) of Wuxi AppTech prior to execution of the in-life portion of the studies. During the study, the care and use of animals were conducted in accordance with the regulations of the Association for Assessment and Accreditation of Laboratory Animal Care (AAALAC). Animals (6-8 weeks) were obtained from Beijing Vital River Laboratory Animal Co., LTD and were allowed to acclimate prior to tumor cell inoculation for efficacy studies and dosing for tolerability studies.

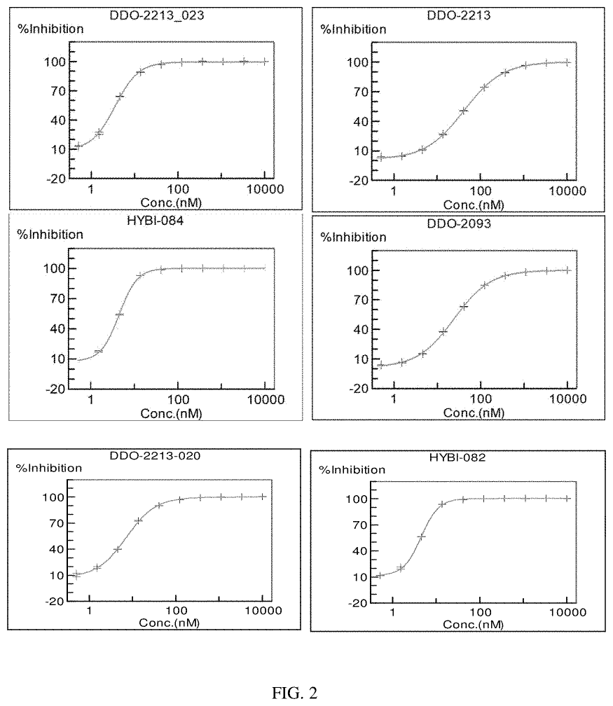

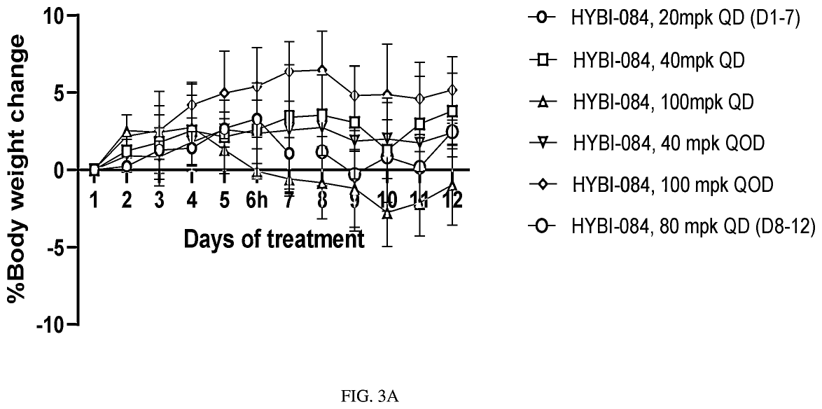

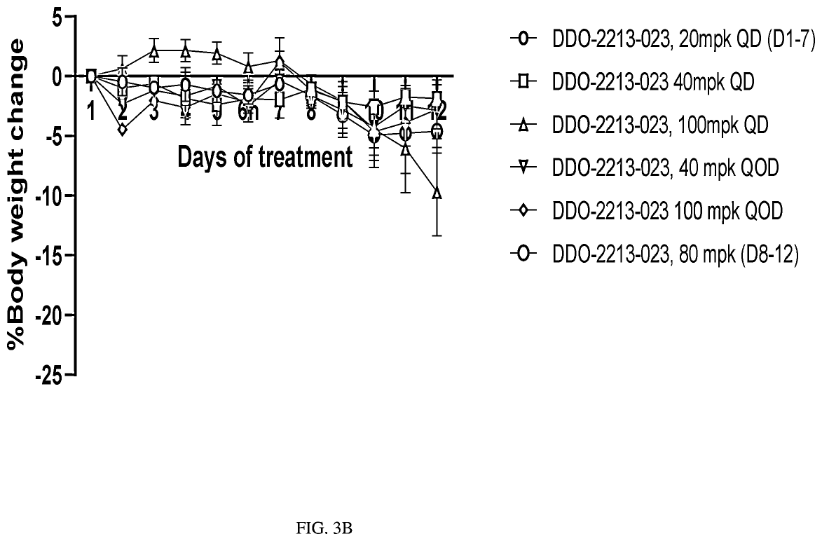

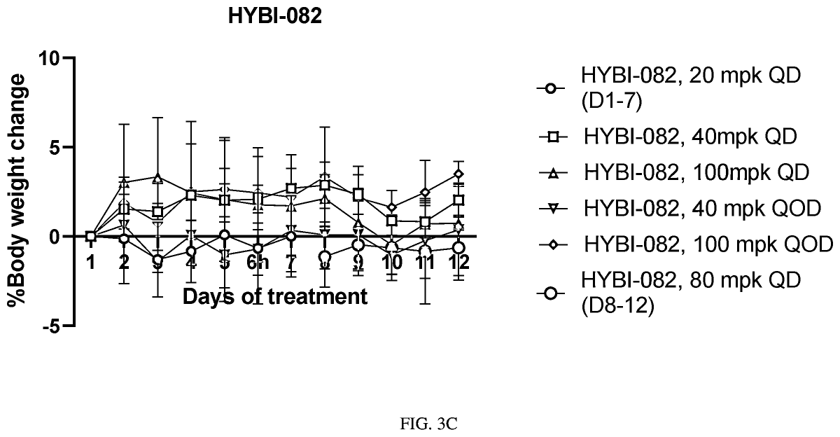

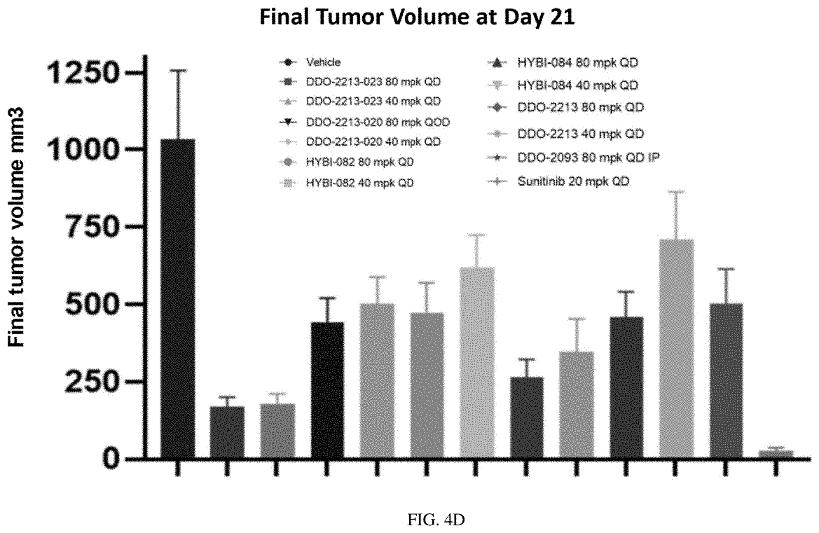

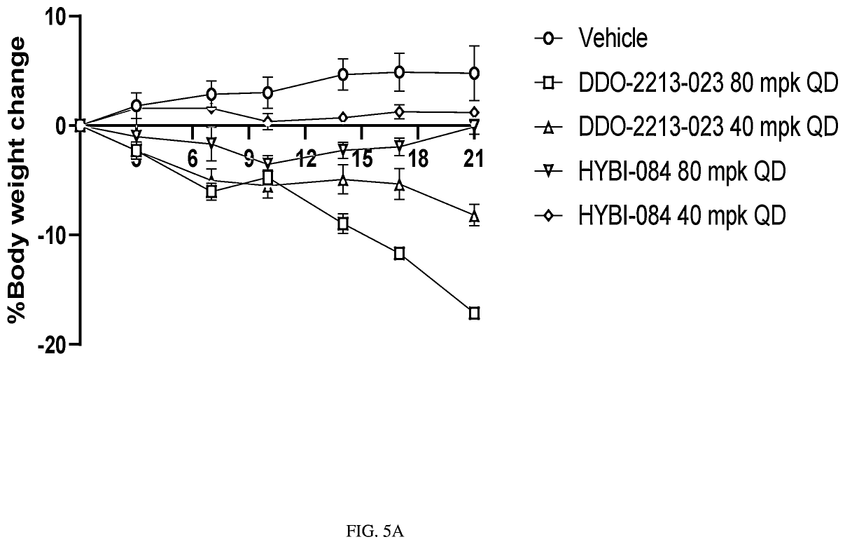

In vivo safety (tolerability) studies: Safety (tolerability) of test articles was evaluated in naïve BALB/c nude mice (n=3/group). All animals were weighed and assigned into groups using randomized block design based upon their body weights to ensure that all the groups are comparable at the baseline. Test articles were formulated in 10% DMSO/10% solutol HS 15/80% water and dosed at 10 mL/kg. Animals were treated with respective doses of test articles for 7 days and body weights measured daily. Any animal exhibiting 20% bodyweight loss at any one day was humanely euthanized and the respective dose considered not tolerated. After 7 days the low dose (20 mg/kg groups) was raised to 80 mg/kg for the remainder of the study to determine if this dose was tolerated. Animals were terminated on day 12. See FIGS. 3A, 3B, 3C and 3D. Also, see FIGS. 5A and 5B.

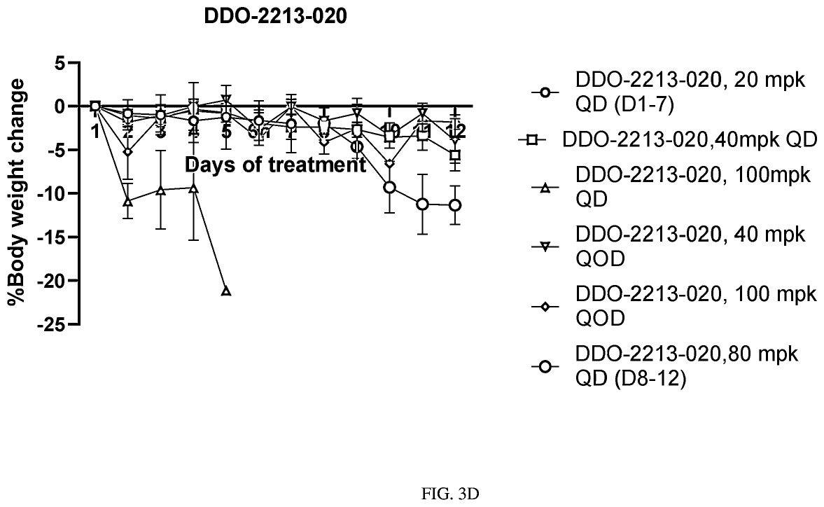

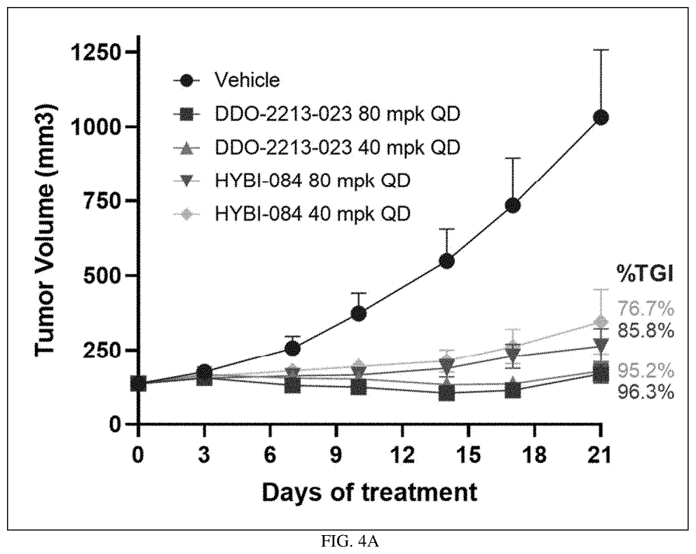

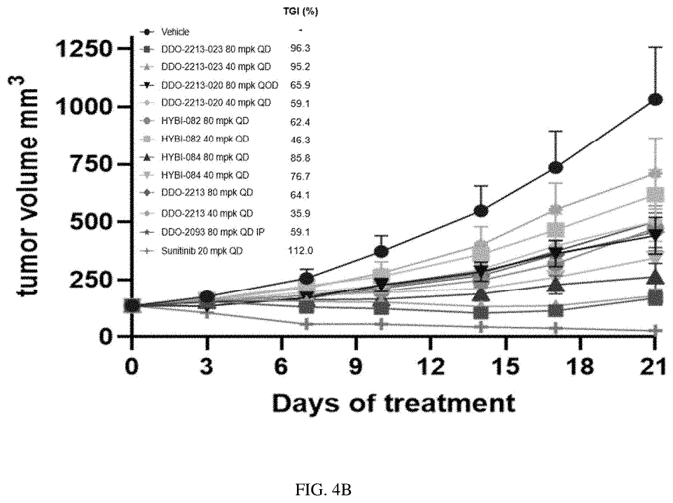

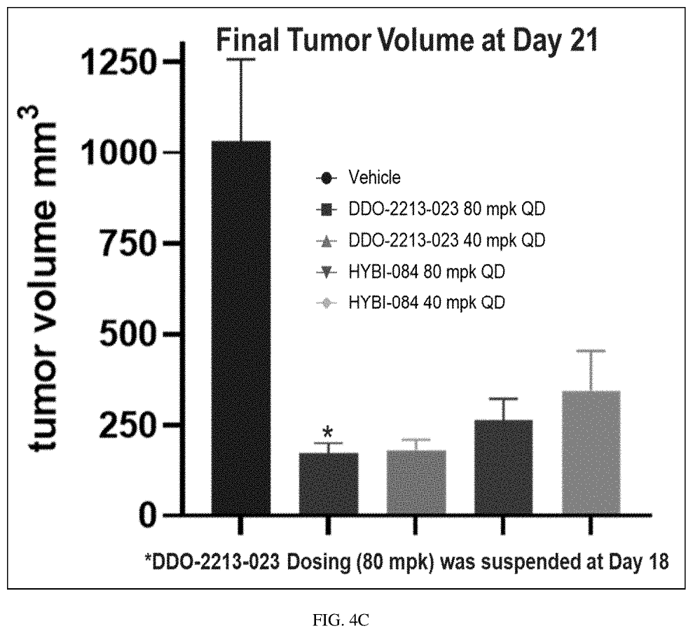

Tumor efficacy studies: MV411 cells were maintained in culture and cells in exponential growth phase were harvested and were quantified by cell counter before tumor inoculation. Each mouse was inoculated subcutaneously at the right flank with MV411 tumor cells (10×10⁶ in 50% matrigel) for tumor development. The animals were randomized and treatment started when the average tumor volume reached 136+/−15 mm³. The date of randomization and treatment initiation was denoted as day 0. Compounds were formulated in 10% DMSO/10% solutol HS 15/80% water and dosed at 10 mL/kg. Each test article was dosed orally at the respective dose daily for 21 days. Body weight and tumor volumes were measured twice per week. Tumor volume was expressed in mm³ using the formula: V=(L×W×W)/2, where V is tumor volume, L is tumor length (the longest tumor dimension) and W is tumor width (the longest tumor dimension perpendicular to L). Upon termination, tumors were collected for further analysis. See FIGS. 4A, 4B, 4C, and 4D.

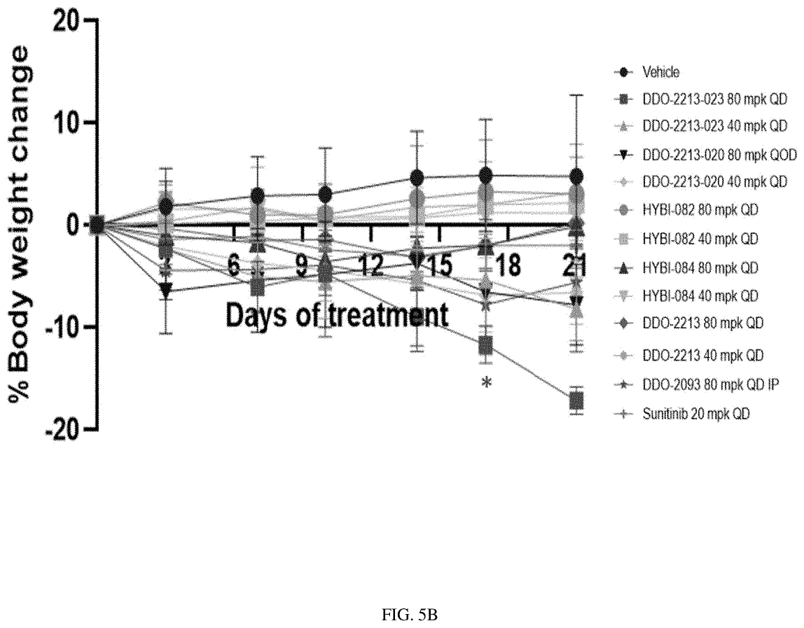

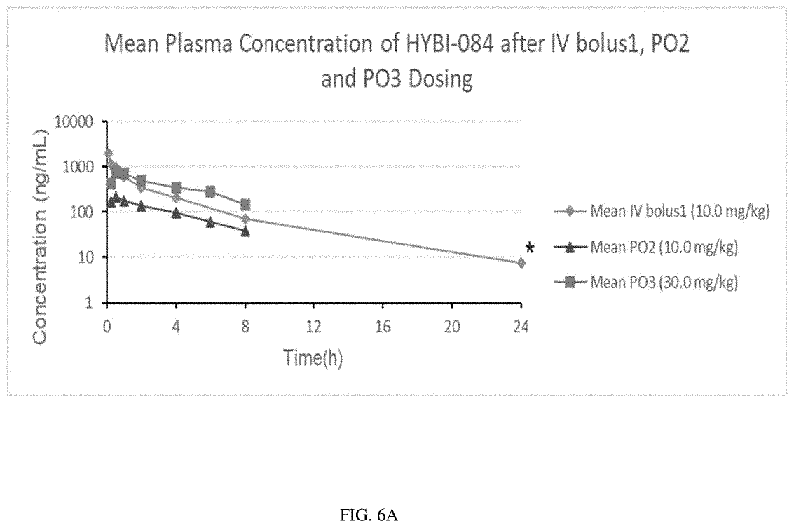

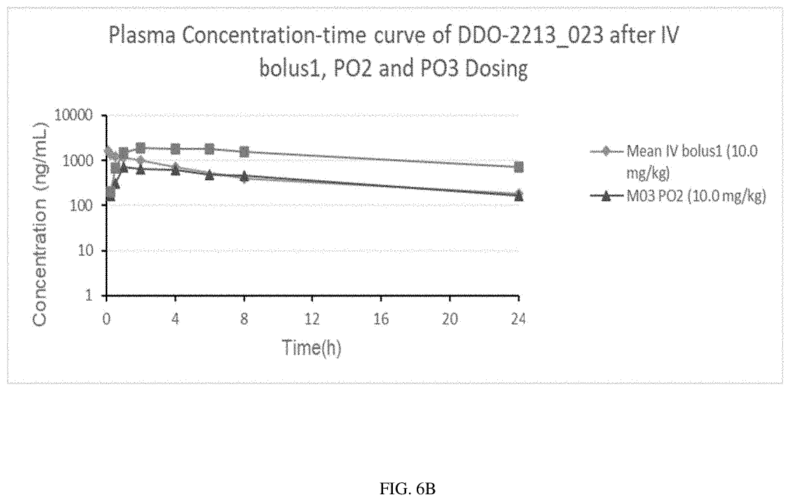

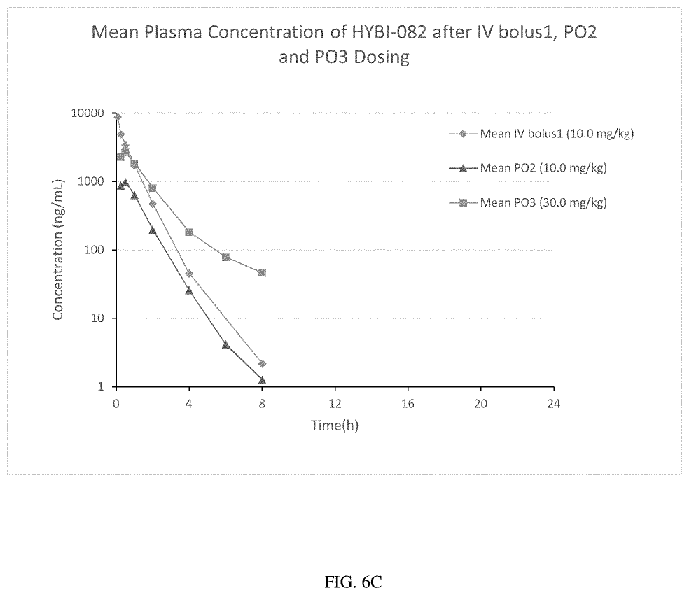

Mouse PK: Animals were dosed with a single intravenous bolus or oral gavage administration in naïve C57/B16 mice (Beijing Vital River Laboratory Animal Co., LTD). Blood samples were collected at the respective sampling time points. The quantification of compound concentrations in mouse or rat plasma was quantified using LC-MS/MS method. Plasma concentration versus time data was analyzed by non-compartmental mode approaches using the WinNonlin software program (version 6.3 or above, Pharsight). Pharmacokinetic parameters, including C₀ (initial concentration after drug administration), CL (serum drug concentration at steady state), V_{dss} (volume of distribution), Cₘₐₓ (maximum peak concentration of the drug), Tₘₐₓ (time required to reach Cₘₐₓ) T_{1/2} (time required for drug concentration to decrease by 50% after administration), AUC₀₋ₗₐₛₜ (area under the concentration-time curve from dosing time t=0 to the time of the last measured concentration), AUC_{0-inf} (total drug exposure across time), MRT₀₋ₗₐₛₜ (mean residence time from dosing time t=0 to the time of the last measured concentration), MRT_{0-inf} f (mean residence time of total drug exposure), bioavailability (%) (the percentage (or the fraction F) of an administered drug dose that reaches the systemic circulation) for IV and PO groups were calculated. See Table 7 and FIGS. 6A, 6B, 6C and 6D.

TABLE 7
Pharmacokinetic properties of compounds of this disclosure in mouse.

| | Mouse Oral PK | | | | | | | |
|---|---|---|---|---|---|---|---|---|
| | Cₘₐₓ (nM) | | T_{1/2} (h) | | AUC₀₋ₗₐₛₜ (nM · h) | | F (%) | |
| | Dose 10 | Dose 30 | Dose 10 | Dose 30 | Dose 10 | Dose 30 | Dose 10 | Dose 30 |
| Compound# | mpk | mpk | mpk | mpk | mpk | mpk | mpk | mpk |
| DDO-2213-023 | 1321 | 3430 | 11.1 | 13.3 | 16415 | 55989 | 82.5 | 93.8 |
| HYBI-084 | 359 | 1276 | 3.1 | 3.5 | 1313 | 5644 | 26.3 | 37.7 |
| DDO-2213-020 | 1673 | 5467 | 3.3 | 2.7 | 7101 | 37047 | 61.5 | 107.0 |
| HYBI-082 | 1672 | 4534 | 0.8 | 2.0 | 2224 | 7605 | 23.3 | 27.5 |

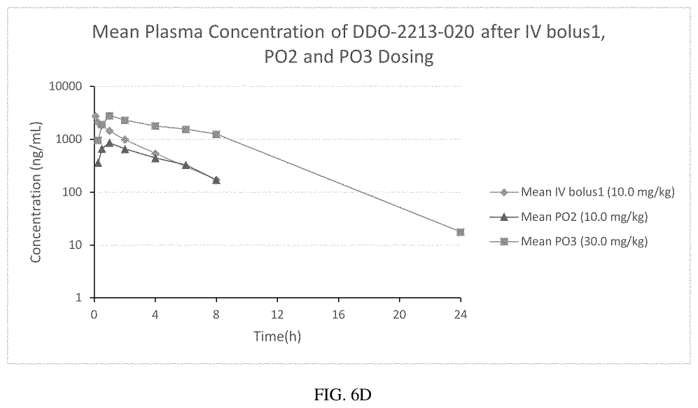

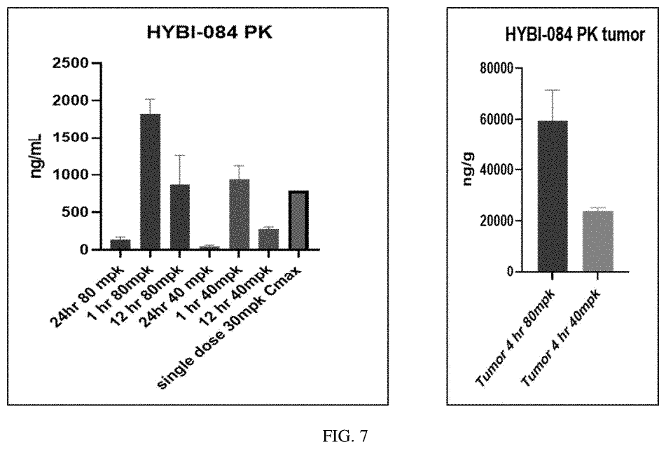

PK from tumor bearing animals: Blood was taken and plasma isolated from tumor bearing animals at respective time points after last administered dose after 22 days of consecutive dosing. Tumors were harvested at 4 hours post last administered dose. Samples were analyzed by LC-MS/MS analysis with Phoenix WinNonlin 6.3 to determine the concentration of compound in plasma or tumor samples. Concentrations were calculated using Linear/log trapezoidal rule. See FIG. 7 and FIG. 8.

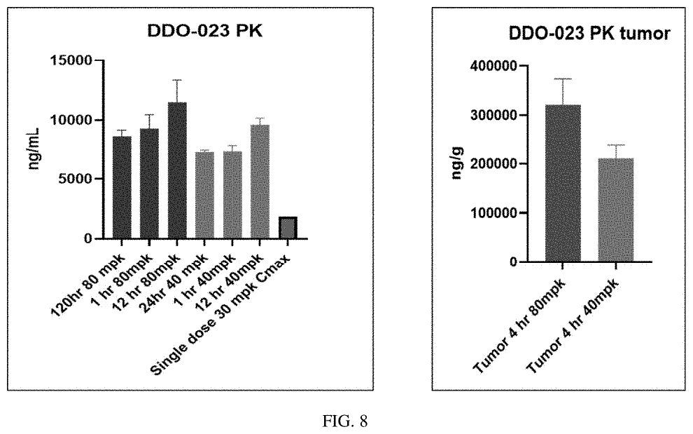

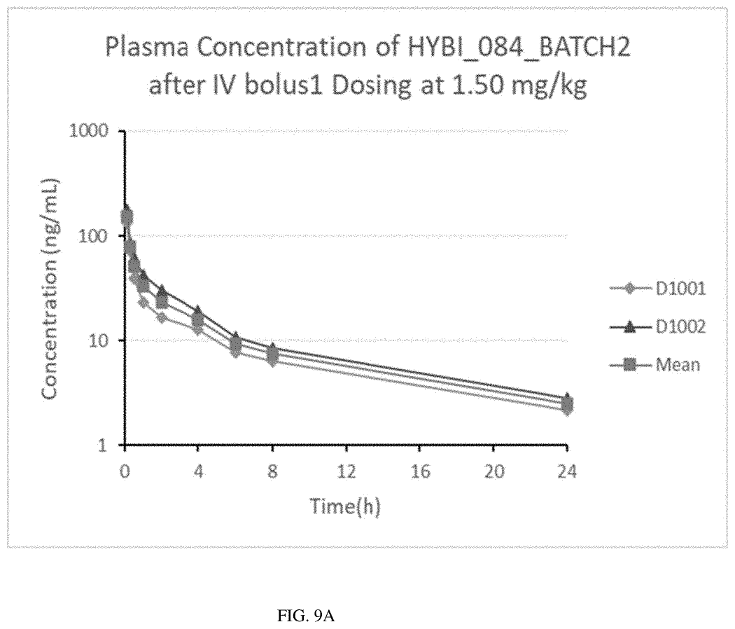

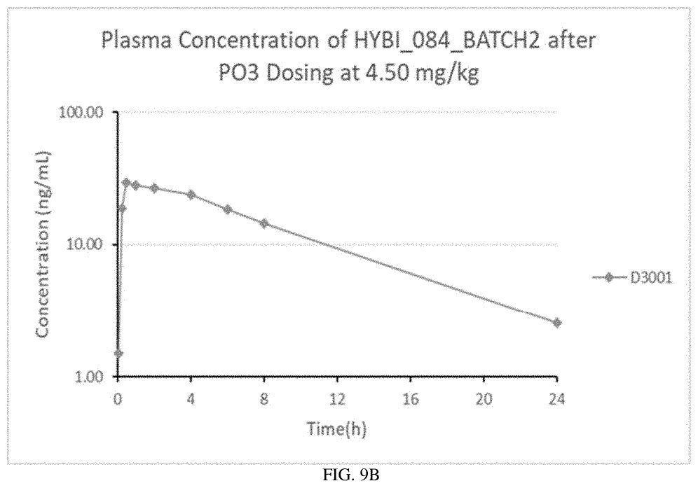

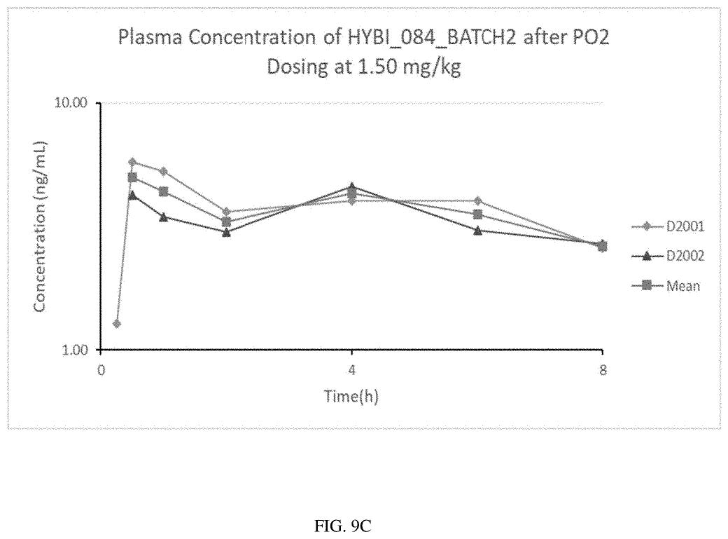

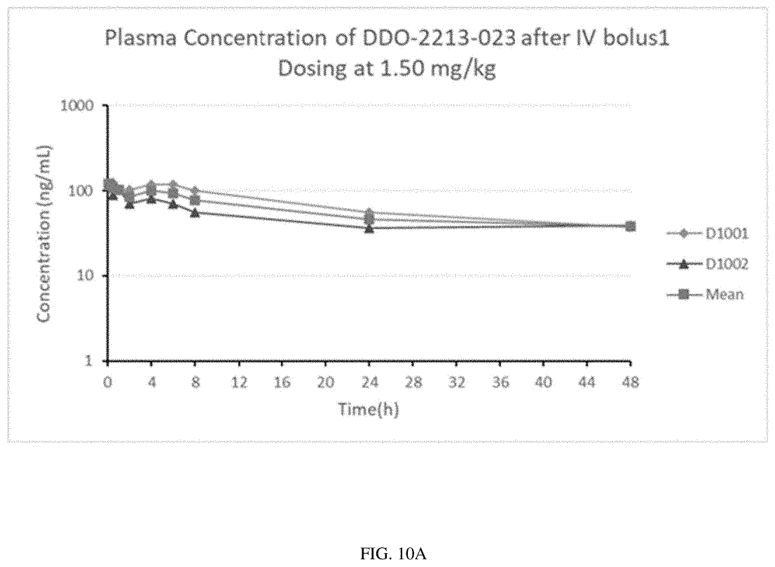

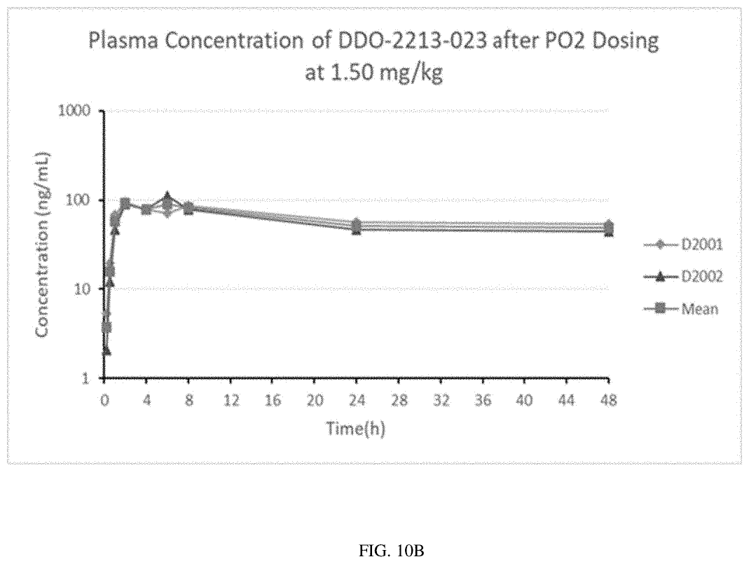

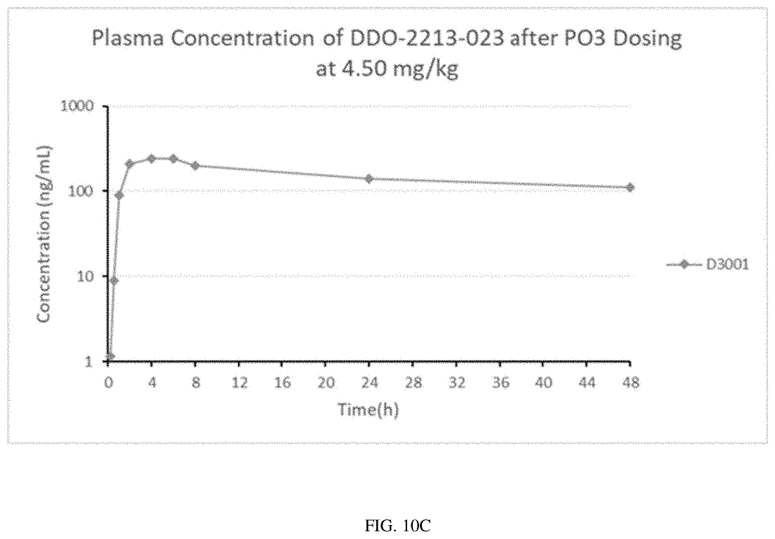

Dog PK: Fasted or fed non-naïve Beagle dogs (Marshall Bioresources, Beijing, China) were administered a single intravenous bolus or oral gavage administration of test compounds at respective doses. Blood samples for plasma isolation were collected from a peripheral vessel, from restrained, non-sedated animals at the respective sampling time points. The quantification of compound concentrations in dog plasma was quantified using LC-MS/MS method. Plasma concentration versus time data was analyzed by non-compartmental mode approaches using the WinNonlin software program (version 6.3 or above, Pharsight). Pharmacokinetic parameters, including C₀, CL, V_{dss}, Cₘₐₓ, Tₘₐₓ T_{1/2}, AUC₀₋ₗₐₛₜ, AUC_{0-inf}, MRT₀₋ₗₐₛₜ, MRT_{0-inf}, bioavailability (%) for IV and PO groups were calculated. The results are summarized in Table 8. See FIGS. 9A, 91B, 9C and FIGS. 10A, 10B, 10C, and 10D.

TABLE 8
Pharmacokinetic properties of representative compounds of this disclosure in dog.

| | Dog Oral PK | | | | | | | |
|---|---|---|---|---|---|---|---|---|
| | Cₘₐₓ (nM) | | T_{1/2} (h) | | AUC₀₋ₗₐₛₜ (nM · h) | | F (%) | |
| | Dose | Dose | Dose | Dose | Dose | Dose | Dose | Dose |
| Compound # | 1.5 mpk | 4.5 mpk | 1.5 mpk | 4.5 mpk | 1.5 mpk | 4.5 mpk | 1.5 mpk | 4.5 mpk |
| DDO-2213-023 | 188 | 445 | 63.6 | 48.2 | 5282 | 13418 | 105.0 | 88.8 |
| HYBI-084 | 8.4 | 47.6 | 7.5 | 6.3 | 46.6 | 459 | 11.4 | 37.7 |

Liver S9 stability assay: Test compounds (1 μM) and positive control were incubated with 5 mM GSH and liver S9 (1 mg protein/ml) from multiple species (including human, monkey, dog, rat and mouse) at 37° C. at 0, 10, 30, and 60 minutes in duplicate. After adding the stop solution, 100 μL of supernatant were analyzed by LC-MS/MS. The % remaining of test compound, T_{1/2} (min), and CLint (μL/min/mg) were calculated and were reported. The Liver stability data are summarized in Tables 9A, 9B3, and 10.

TABLE 9A
Liver S9 stability of compounds of this disclosure in multiple species.

| | % Remaining after 60 min incubation | | | | T_{1/2} (min) | | | |
|---|---|---|---|---|---|---|---|---|
| Compd# | Human | Rat | Dog | Mouse | Human | Rat | Dog | Mouse |
| DDO-2213-023 | 81.8 | 101 | 112 | 71.5 | >145 | >145 | >145 | 126 |
| DDO-2213-020 | 92.1 | 88.8 | 78 | 93.4 | >145 | >145 | >145 | >145 |
| HYBI-082 | 107 | 98.3 | 87.3 | 87.7 | >145 | >145 | >145 | >145 |
| HYBI-084 | 85.1 | 80.1 | 92.5 | 110 | >145 | >145 | >145 | >145 |

TABLE 9B
Liver S9 stability of compounds of this disclosure in multiple species.

| | % Remaining after 60 min incubation | | | | CLint (μL/min/mg) | | | |
|---|---|---|---|---|---|---|---|---|
| Compd# | Human | Rat | Dog | Mouse | Human | Rat | Dog | Mouse |
| DDO-2213-023 | 81.8 | 101 | 112 | 71.5 | <4.8 | <4.8 | <4.8 | 5.5 |
| DDO-2213-020 | 92.1 | 88.8 | 78 | 93.4 | <4.8 | <4.8 | <4.8 | <4.8 |
| HYBI-082 | 107 | 98.3 | 87.3 | 87.7 | <4.8 | <4.8 | <4.8 | <4.8 |
| HYBI-084 | 85.1 | 80.1 | 92.5 | 110 | <4.8 | <4.8 | <4.8 | <4.8 |

TABLE 10
Metabolite ID in Liver S9 and plasma, plasma protein binding, and
hERG IC₅₀ (μM) of representative compounds of this disclosure.

| | | | Relative Abundance | | | | | Metabolic |
|---|---|---|---|---|---|---|---|---|
| Met in S9 | [M + H]⁺ | RT | (UV Peak Area % Total) | | | | | Pathways |
| Code | m/z | (min) | Mouse | Rat | Dog | Monkey | Human | Key ID |
| M1 | 618.2503 | 7.35 | ND | ND | ND | 2.24 | ND | A |
| M2 | 632.2666 | 7.55 | 14.51 | 4.85 | 1.83 | 22.16 | ND | B |
| M4 | 588.2394 | 10.38 | 1.12 | 0.60 | 3.35 | 6.37 | 0.67 | C |
| M5 | 602.2573 | 10.51 | 2.62 | 2.29 | 11.31 | 4.56 | 2.87 | D |
| M6 | 602.2565 | 10.62 | 14.9 | 17.21 | 8.04 | 17.49 | 13.48 | D |
| HBI-2375 | 616.2720 | 10.75 | 63.02 | 71.86 | 70.71 | 34.68 | 78.70 | NA |
| M7 | 632.2655 | 11.77 | 1.53 | 1.53 | 2.90 | 6.63 | 3.11 | B |
| M8 | 604.2363 | 13.06 | ND | ND | 0.18 | 1.1 | ND | E |
| M9 | 618.2495 | 13.23 | 0.26 | 0.40 | 0.36 | 2.21 | + | A |
| M10 | 534.1817 | 13.49 | 2.03 | 1.26 | 1.32 | 1.30 | 1.18 | F |
| M11 | 616.2366 | 14.10 | + | ND | ND | 1.27 | + | G |
| Met in | | | Relative Abundance | | | | | Metabolic |
| Plasma | [M + H]⁺ | RT | (UV Peak Area % Total) | | | | | Pathways |
| Code | m/z | (min) | Mouse | Rat | Dog | Monkey | Human | Key ID |
| M3 | 778.3239 | 9.23 | 5.42 | 4.76 | 2.05 | 1.30 | 1.58 | H |
| HBI-2375 | 616.2734 | 10.72 | 92.78 | 93.47 | 97.31 | 97.58 | 97.20 | NA |
| M10 | 534.1829 | 13.46 | 1.80 | 1.77 | 0.64 | 1.11 | 1.23 | F |

| | |
|---|---|
| hERG IC₅₀ (μM) | 22.24 |
| Plasma Protein Binding | High(>93% H, R, M) |
| (H, R, D, CM, M) | Moderate (>76% D, CM) |

Key for Table 10:

| Key ID | Pathway | Key ID | Pathway |
|---|---|---|---|
| A | N-Dealkylation and acetylation | F | Hydrolysis |
| | (P − C₃H₄ + C₂H₂O) | | (P + H₂O − C₅H₁₂N₂) |
| B | Mono-oxygenation | G | N-Demethylation, mono- |
| | (P + O) | | oxygenation and dehydrogenation |
| | | | (P − CH₂ + O − 2H) |
| C | Di-N-demethylation (P − 2CH₂) | H | Glucose conjugation (P + C₆H₁₀O₅) |
| D | N-Demethylation (P − CH₂) | NA | Not Applicable |
| E | Di-demethylation and mono- | | |
| | oxygenation (P − 2CH₂ + O) | | |

WBS (whole blood stability) assay: The blood samples were collected freshly from multiple species including human, monkey, dog, rat and mouse. 2 μM of test compound and positive control were incubated with the whole blood at 37° C. at 0, 10, 30, 60, and 120 minutes in duplicate. After adding the stop solution, 200 μL of supernatant were analyzed by LC-MS/MS. The % remaining of test compound and T_{1/2} (min) were calculated and reported. These results are summarized in Table 11.

TABLE 11
Whole blood stability of this disclosure from multipl species.

| | % Remaining after | | | | | | | |
|---|---|---|---|---|---|---|---|---|
| | 60 min incubation | | | | T_{1/2} (min) | | | |
| Compd# | Human | Rat | Dog | Mouse | Human | Rat | Dog | Mouse |
| DDO-2213-023 | 103 | 103 | 97.3 | 103 | 525 | >144.5 | >144.5 | >144.5 |
| DDO-2213-020 | 103 | 1 | 101 | 104 | 300 | >144.5 | >144.5 | >144.5 |
| HYBI-082 | 100 | 99.6 | 90.6 | 82.4 | 716.00 | >144.5 | >144.5 | >144.5 |
| HYBI-084 | 110 | 109 | 94.6 | 108 | 616.00 | >144.5 | >144.5 | >144.5 |

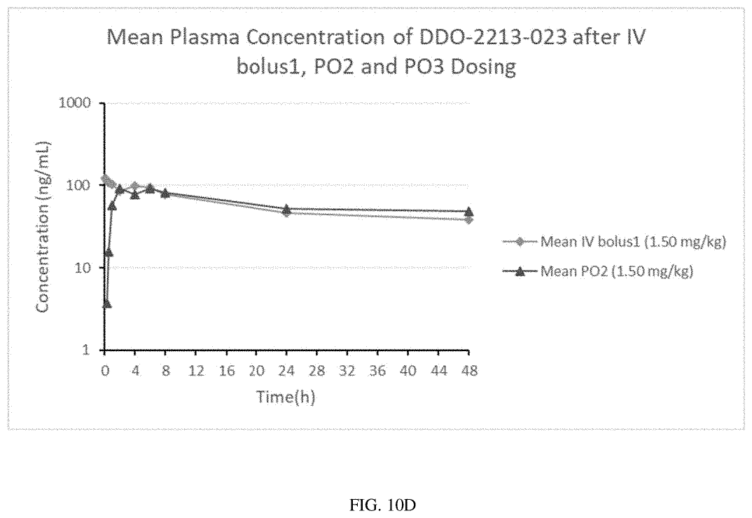

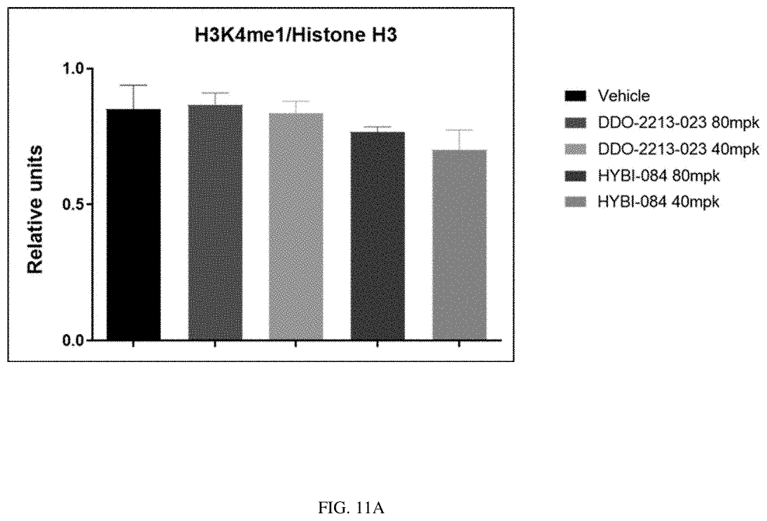

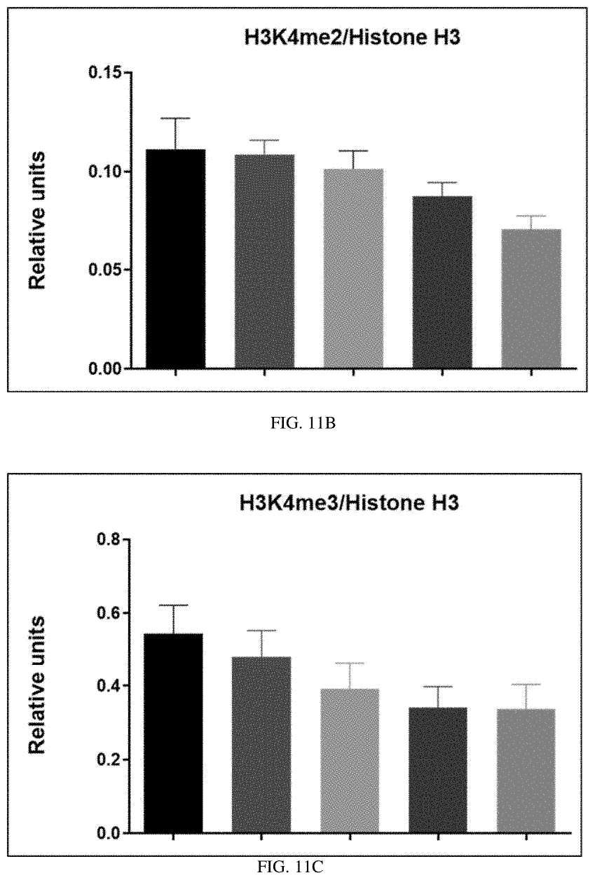

Inhibition of HMT2&3 in MV 411 tumors: Histone methylation analysis with MesoScale Discovery (MSD) ELISA method in MV4-11 tumors: Capture antibody, Anti-Histone antibody clone H11-4 (MAB3422) was purchased from EMD-Millipore. Detection antibodies, H3 (D1H2) XP® Rabbit mAb (4499) was purchased from CST. Methyl-Histone H3 (Lys4) Recombinant Polyclonal Antibody (710795), Di-Methyl-Histone H3 (Lys4) Recombinant Polyclonal Antibody (710796) and Tri-Methyl-Histone H3 (Lys4) Polyclonal Antibody (PA5-27029) were purchased from Thermo Scientific. Anti Rabbit Antibody Goat SULFO-TAG Labeled (R32AB-1) was purchased from MSD. MV4-11 tumor cell lysate was extracted with RIPA buffer with protease inhibitors and PMSF according to MSD instructions. Plates were coated and tumor lysates incubated with the antibodies for ELISA according to MSD instructions. Signal for each well was read on MESO SECTOR S 600 and the ratio of Mono, Di, or Tri methyl histone H3 to total H3 was analyzed. See FIGS. 11A-C.

EXAMPLE 3: PK profile of HBI-2375 (compound of Formula (IIIb) (HYBI-084) in plasma, brain, and cerebrospinal fluid (CSF) in SD rats: This study was conducted in accordance with the Study Protocol and Pharmaron (Ningbo) TSP's Standard Operating Procedures (SOPs) following all regulatory protocols. This study was conducted under non-GLP environment.

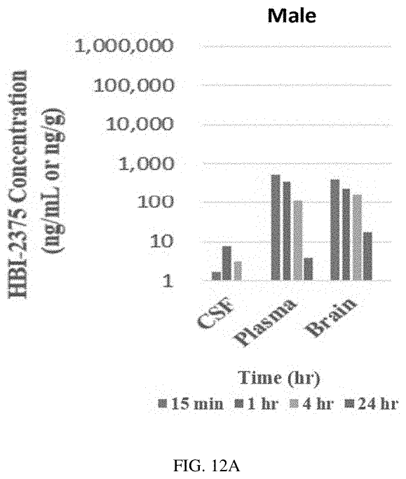

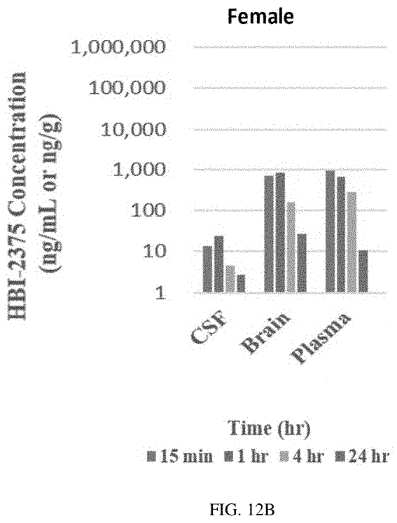

Following a single oral gavage administration of HBI-2375/HYBI-084 at 40 mg/kg in SD rats, the maximal concentrations of HBI-2375 in plasma for males and females were observed at 15 min post-dose. The maximal concentrations of HBI-2375 in CSF for males and females were at 1 hr post-dose. The maximal concentrations of HBI-2375 in brain were at 15 min post-dose for males and at 1 hr post-dose for females. The mean values of AUCₗₐₛₜ for HBI-2375 from high to low were: brain>plasma>CSF for males, and plasma>brain>CSF for females. See FIGS. 12A-B.

The tissue to plasma ratios of AUCₗₐₛₜ for HBI-2375 are summarized in the Table 12 below. The exposure of HBI-2375 in CSF was lower than that in plasma, the ratios were 0.011 for males and 0.0338 for females. The exposure in brain was close to that in plasma, the ratios were 1.31 for males and 0.928 for females.

TABLE 12
Summary of female to male ratios of AUCₗₐₛₜ

AUCₗₐₛₜ Ratio

| | |
|---|---|
| Tissue/Plasma* | C_{24 hr}/Cₘₐₓ Ratio (%) |

| Matrix | Male | Female | Female/Male | Male | Female |
|---|---|---|---|---|---|
| CSF | 0.0111 | 0.0338 | 6.66 | NC | 11.6 |
| Plasma | — | — | 2.18 | 0.697 | 1.17 |
| Brain | 1.31 | 0.928 | 1.54 | 4.18 | 3.24 |

*: The plasma density was calculated at 1 g/mL.
NC: Not calculated.
“—”: Not applicable.

As shown in Table 13, following single oral gavage of HBI-2375 at 40 mg/kg, the Tₘₐₓ was 15 min in male and female SD rat plasma. The mean values of the maximum concentration (Cₘₐₓ) for HBI-2375 in SD rat plasma were 544 ng/mL for males and 934 ng/mL for females. The maximal concentrations of HBI-2375 in CSF for males and females were at 1 hr post-dose. The maximal concentrations of HBI-2375 in brain were at 15 min post-dose for males and at 1 hr post-dose for females. The mean values of Cₘₐₓ for HBI-2375 in CSF were 7.48 ng/mL for males and 24.0 for females. The mean values of Cₘₐₓ for HBI-2375 in brain were 413 ng/g for males, and 833 ng/g for females, respectively.

As shown in Table 13, following single oral gavage of HBI-2375 at 40 mg/kg, the MRTₗₐₛₜ for HBI-2375 in SD rat plasma were 4.50 hr for males and 5.11 hr for females. The MRTₗₐₛₜ CSF were 2.00 hr for males and 8.41 hr for females. The mean values of MRTₗₐₛₜ in brain were 7.00 hr for males, and 5.81 hr for females.

As shown in Table 13, following single oral gavage of HBI-2375 at 40 mg/kg, the mean exposures (AUCₗₐₛₜ) for HBI-2375 in SD rat plasma were 1679 hr*ng/mL for males and 3659 hr*ng/mL for females. The mean values of AUCₗₐₛₜ in CSF were 18.6 hr*ng/mL for males and 124 hr*ng/mL for females. The mean values of AUCₗₐₛₜ in brain were 2199 hr*ng/mL for males and 3394 hr*ng/mL for females. The values of AUCₗₐₛₜ for HBI-2375 from high to low were: brain>plasma>CSF for males, and plasma>brain>CSF for females.

TABLE 13
PK Parameters of HBI-2375 in Plasma, CSF, and Brain Tissues
of SD Rats Following Single-Dose Oral Administration at 40 mg/kg

| | | Tₘₐₓ | Cₘₐₓ (ng/mL | AUCₗₐₛₜ (hr*ng/ml | MRTₗₐₛₜ |
|---|---|---|---|---|---|
| Matrix | Sex | (hr) | or ng/g) | or hr*ng/g) | (hr) |
| CSF | Male | 1 | 7.48 | 18.6 | 2.00 |
| | Female | 1 | 24.0 | 124 | 8.41 |
| Plasma | Male | 0.25 | 544 | 1679 | 4.50 |
| Mean | Female | 0.25 | 934 | 3659 | 5.11 |
| | | — | 739 | 2669 | 4.80 |
| Brain | Male | 0.25 | 413 | 2199 | 7.00 |
| Mean | Female | 1 | 833 | 3394 | 5.81 |
| | | — | 623 | 2796 | 6.40 |

The tissue to plasma ratios of AUCₗₐₛₜ are summarized in Table 13. The exposure of HBI-2375 in CSF was lower than that in plasma, the ratios were 0.011 for males and 0.0338 for females. The exposure in brain was close to that in plasma, the ratios were 1.31 for males and 0.928 for females.

The female to male ratios of AUCₗₐₛₜ are summarized in Table 14. The exposure of HBI-2375 in CSF, plasma and brain of females was higher than that of males, the ratios were 6.66, 2.18, 1.54 respectively.

TABLE 14
The Ratios of AUCₗₐₛₜ for HBI-2375 Following Single-Dose Oral
Administration of HBI-2375 at 40 mg/kg in SD Rats

| | AUCₗₐₛₜ Ratio | | | | |
|---|---|---|---|---|---|
| | Tissue/Plasma* | | Female/ | C_{24 hr}/Cₘₐₓ Ratio (%) | |
| Matrix | Male | Female | Male | Male | Female |
| CSF | 0.0111 | 0.0338 | 6.66 | NC | 11.6 |
| Plasma | — | — | 2.18 | 0.697 | 1.17 |
| Brain | 1.31 | 0.928 | 1.54 | 4.18 | 3.24 |

*: The plasma density was calculated at 1 g/mL.
NC: Not calculated.
“—”: Not applicable.

Following a single oral gavage administration of HBI-2375 at 40 mg/kg in SD rats, concentrations of HBI-2375 in plasma, brain, and CSF were determined by LC-MS/MS methods. During the in-life period, no abnormalities were observed for all animals. The maximal concentrations of HBI-2375 in plasma for males and females were observed at 15 min post-dose. The maximal concentrations of HBI-2375 in CSF for males and females were at 1 hr post-dose. The maximal concentrations of HBI-2375 in brain were at 15 min post-dose for males and at 1 hr post-dose for females.

The exposure of HBI-2375 in CSF was lower than that in plasma, the ratios were 0.011 for males and 0.0338 for females. The exposure in brain was close to that in plasma, the ratios were 1.31 for males and 0.928 for females. The exposure of HBI-2375 in CSF, plasma and brain of females was higher than that of males, the ratios were 6.66, 2.18, and 1.54 respectively. The values of AUClast for HBI-2375 from high to low were: brain>plasma>CSF for male SD rats, and plasma>brain>CSF for female SD rats.

In conclusion, after a single oral administration of HBI-2375 at 40 mg/kg in SD rats, HBI-2375 was distributed to CSF and brain, and the exposure lasted for 24 hours. The exposure in brain was close to that in plasma, whereas the exposures in CSF were lower than that in plasma.

EXAMPLE 4: PK profile of compound of Formula (IIIb) (HBI-2375, HYBI-84) in plasma and brain in C57BL/6J mice: This study was conducted in accordance with the Study Protocol and Pharmaron (Ningbo) TSP's Standard Operating Procedures (SOPs) following all regulatory protocols. This study was conducted under non-GLP environment.

Following single oral gavage of HBI-2375 at 40 mg/kg in C57BL/6J mice, the Tₘₐₓ of HBI-2375 was 30 min, Cₘₐₓ was 1139 ng/mL, AUCₗₐₛₜ was 6598 hr*ng/mL, MRTₗₐₛₜ was 5.26 hr in plasma. The Tₘₐₓ was 4 hr, Cₘₐₓ was 180 ng/g, AUCₗₐₛₜ was 2029 hr*ng/g, MRTₗₐₛₜ was 8.15 hr in brain. Details are shown in Table 15. Also see FIG. 16A-B.

Following single oral gavage of HBI-2375 at 80 mg/kg in C57BL/6J mice, the Tₘₐₓ of HBI-2375 was 30 min, Cₘₐₓ was 3973 ng/mL, AUCₗₐₛₜ was 15604 hr*ng/mL, MRTₗₐₛₜ was 5.16 hr in plasma. The Tₘₐₓ was 4 hr, Cₘₐₓ was 528 ng/g, AUCₗₐₛₜ was 5844 hr*ng/g, MRTₗₐₛₜ was 8.21 hr in brain. Details are shown in Table 15. Also see FIG. 16A-B.

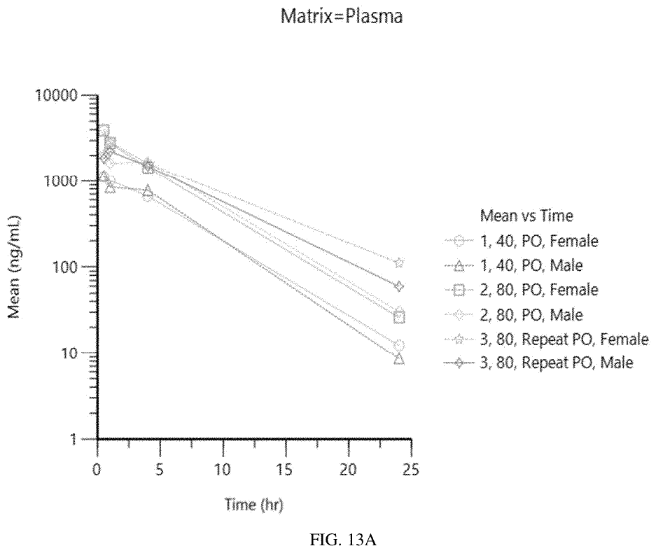

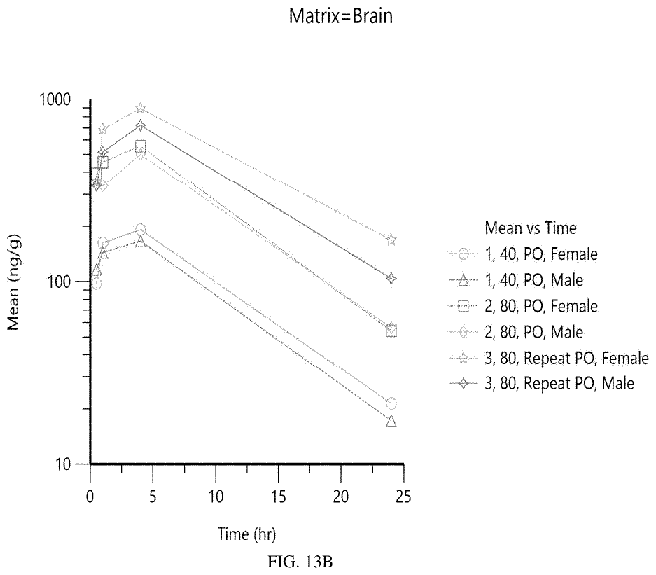

Following 7-day repeat oral gavage of HBI-2375 at 80 mg/kg in C57BL/6J mice, the Tₘₐₓ of HBI-2375 was 1 hr, Cₘₐₓ was 2533 ng/mL, AUCₗₐₛₜ was 17709 hr*ng/mL in plasma. The Tₘₐₓ was 4 hr, Cₘₐₓ was 807 ng/g, AUCₗₐₛₜ was 10001 hr*ng/g in brain. Details are shown in Table 15. Also see FIGS. 13A-B.

TABLE 15
PK Parameters of HBI-2375 in Plasma and Brain Following
Oral Administration of HBI-2375 in C57BL/6J Mice

| | Dose | Adminis- | | | | Cₘₐₓ | AUCₗₐₛₜ | |
|---|---|---|---|---|---|---|---|---|
| | Level | tration | | | Tₘₐₓ | (ng/ml | (hr*ng/mL | MRTₗₐₛₜ |
| Group | (mg/kg) | Route | Matrix | Sex | (hr) | or ng/g) | or hr*ng/g) | (hr) |
| 1 | 40 | PO | Plasma | Male | 0.5 | 1153 | 6636 | 5.22 |
| | | | | Female | 0.5 | 1125 | 6560 | 5.30 |
| | | | Mean | | — | 1139 | 6598 | 5.26 |
| | | | Brain | Male | 4 | 167 | 1879 | 8.05 |
| | | | | Female | 4 | 192 | 2179 | 8.25 |
| | | | Mean | | — | 180 | 2029 | 8.15 |
| 2 | 80 | PO | Plasma | Male | 0.5 | 4040 | 15401 | 5.46 |
| | | | | Female | 0.5 | 3907 | 15807 | 4.86 |
| | | | Mean | | — | 3973 | 15604 | 5.16 |
| | | | Brain | Male | 4 | 500 | 5559 | 8.40 |
| | | | | Female | 4 | 556 | 6129 | 8.01 |
| | | | Mean | | — | 528 | 5844 | 8.21 |
| 3 | 80 | Repeat | Plasma | Male | 1 | 2230 | 15960 | NC |
| | | PO | | Female | 1 | 2837 | 19459 | NC |
| | | | Mean | | — | 2533 | 17709 | — |
| | | | Brain | Male | 4 | 722 | 8555 | NC |
| | | | | Female | 4 | 892 | 11446 | NC |
| | | | Mean | | — | 807 | 10001 | — |

NC: Not calculated.
“—”: Not applicable.

Following single oral gavage administration of HBI-2375 at 40 mg/kg, 80 mg/kg, and 7-day repeat oral gavage administration at 80 mg/kg in C57BL/6J mice, the mean concentrations of HBI-2375 at 24 hr post-dose in plasma were 0.748-1.10%, 0.671-0.757%, 2.68-3.94% of Cₘₐₓ respectively. The mean concentration of HBI-2375 at 24 hr post-dose in brain were 10.4-11.1%, 9.71-11.2%, 14.4-18.9% of Cₘₐₓ respectively. Details are shown in Table 16.

Following single oral gavage administration of HBI-2375 at 40 mg/kg, 80 mg/kg, and 7-day repeat oral gavage administration at 80 mg/kg in C57BL/6J mice, the exposure of HBI-2375 in brain was lower than that in plasma, the ratios were 0.283, 0.361, 0.536 respectively for males, 0.332, 0.388, 0.588 respectively for females. Details are shown in Table 16.

TABLE 16
The Ratios of AUCₗₐₛₜ and C₂₄ₕᵣ/Cₘₐₓ for HBI-2375 in Plasma and Brain
Following Oral Administration of HBI-2375 in C57BL/6J Mice

| | Dose | Admini- | | AUCₗₐₛₜ Ratio | | | C₂₄ₕᵣ/Cₘₐₓ | |
|---|---|---|---|---|---|---|---|---|
| Group | Level | stration | | Brain/Plasma* | | Female/ | Ratio (%) | |
| No. | (mg/kg) | Route | Matrix | Male | Female | Male | Male | Female |
| 1 | 40 | PO | Plasma | — | — | 0.989 | 0.748 | 1.10 |
| | | | Brain | 0.283 | 0.332 | 1.16 | 10.4 | 11.1 |
| 2 | 80 | PO | Plasma | — | — | 1.03 | 0.757 | 0.671 |
| | | | Brain | 0.361 | 0.388 | 1.10 | 11.2 | 9.71 |
| 3 | 80 | Repeat PO | Plasma | — | — | 1.22 | 2.68 | 3.94 |
| | | | Brain | 0.536 | 0.588 | 1.34 | 14.4 | 18.9 |

*The plasma density was calculated at 1 g/mL.
“—”: Not applicable.

As shown in Table 17, following single oral gavage administration of HBI-2375 at 40 mg/kg, 80 mg/kg, and 7-day repeat oral gavage administration at 80 mg/kg in C57BL/6J mice, the exposure of HBI-2375 in plasma of females was close to that in males (the Cₘₐₓ ratios were 0.967-1.27, AUCₗₐₛₜ ratios were 0.989-1.22), indicating there were no gender-related differences in the exposure of HBI-2375 in plasma of C57BL/6J mice.

As shown in Table 17, following single-dose oral administration of HBI-2375 at 40 mg/kg and 80 mg/kg in C57BL/6J mice, the increase of AUCₗₐₛₜ for HBI-2375 in plasma was close to the dose-proportional increase, the ratios were 2.32-2.41.

As shown in Table 17, following 7-day repeat oral administration of HBI-2375 at 80 mg/kg in C57BL/6J mice, the AUCₗₐₛₜ of HBI-2375 in plasma were closed to that from single-dose at 80 mg/kg, the ratios were 1.04-1.23. This result indicates that there was no obvious drug accumulation in the plasma of C57BL/6J mice after 7-day consecutive dosing of HBI-2375 at 80 mg/kg.

TABLE 17
The Ratios of Cₘₐₓ and AUCₗₐₛₜ for HBI-2375 in Plasma
Following Oral Administration of HBI-2375 in C57BL/6J Mice

| | Dose | Admini- | | | | | |
|---|---|---|---|---|---|---|---|
| | Level | stration | | | Dose | Cₘₐₓ | AUCₗₐₛₜ |
| Group | (mg/kg) | Route | Matrix | Sex | Ratio | Ratio | Ratio |
| 1 | 40 | PO | | Female/Male | | 0.976 | 0.989 |
| 2 | 80 | PO | | Female/Male | 1 | 0.967 | 1.03 |
| 3 | 80 | Repeat PO | | Female/Male | | 1.27 | 1.22 |
| | | | Plasma | Male | | 3.50 | 2.32 |
| 2 vs 1 | 80 vs 40 | PO vs PO | | | 2 | | |
| | | | | Female | | 3.47 | 2.41 |
| 3 vs 2 | 80 vs 80 | Repeat PO vs | | Male | 1 | 0.552 | 1.04 |
| | | PO | | Female | | 0.726 | 1.23 |

As shown in Table 18, following single oral gavage administration of HBI-2375 at 40 mg/kg, 80 mg/kg, and 7-day repeat oral gavage administration at 80 mg/kg in C57BL/6J mice, the exposure of HBI-2375 in brain of females was close to that in males (the Cₘₐₓ ratios were 1.11-1.24, AUCₗₐₛₜ ratios were 1.10-1.34), indicating there were no gender-related differences in the exposure of HBI-2375 in brain of C57BL/6J mice.

As shown in Table 18, following single-dose oral administration of HBI-2375 at 40 mg/kg and 80 mg/kg in C57BL/6J mice, the increase of AUCₗₐₛₜ for HBI-2375 in brain was close to the dose-proportional increase, the ratios were 2.81-2.96.

As shown in Table 18, following 7-day repeat oral administration of HBI-2375 at 80 mg/kg in C57BL/6J mice, the exposure of HBI-2375 in brain were closed to that from single-dose at 80 mg/kg, the Cₘₐₓ ratios were 1.44-1.60, AUCₗₐₛₜ ratios were 1.54-1.87. This result indicates that there was no obvious drug accumulation in the brain of C57BL/6J mice after 7-day consecutive dosing of HBI-2375 at 80 mg/kg.

TABLE 18
The Ratios of Cₘₐₓ and AUCₗₐₛₜ for HBI-2375 in Brain
Following Oral Administration of HBI-2375 in C57BL/6J Mice

| | Dose | Admini- | | | | | |
|---|---|---|---|---|---|---|---|
| | Level | stration | | | Dose | Cₘₐₓ | AUCₗₐₛₜ |
| Group | (mg/kg) | Route | Matrix | Sex | Ratio | Ratio | Ratio |
| 1 | 40 | PO | | Female/Male | 1 | 1.15 | 1.16 |
| 2 | 80 | PO | | Female/Male | | 1.11 | 1.10 |
| 3 | 80 | Repeat PO | | Female/Male | | 1.24 | 1.34 |
| 2 vs 1 | 80 vs 40 | PO vs PO | Brain | Male | 2 | 3.00 | 2.96 |
| | | | | Female | | 2.89 | 2.81 |
| 3 vs 2 | 80 vs 80 | Repeat PO | | Male | 1 | 1.44 | 1.54 |
| | | vs PO | | Female | | 1.60 | 1.87 |

Following single oral gavage administration of HBI-2375 at 40 mg/kg, 80 mg/kg, and 7-day repeat oral gavage administration at 80 mg/kg in C57BL/6J mice, concentrations of HBI-2375 in plasma and brain were determined by LC-MS/MS methods. During the in-life period, no abnormalities were observed for all animals. No gender-related differences in the exposure of HBI-2375 in plasma and brain were founded. The maximal concentrations of HBI-2375 in plasma were observed at 30 min, 30 min, and 1 hr post-dose respectively. The maximal concentrations of HBI-2375 in brain were all observed at 4 hr post-dose. The mean concentration of HBI-2375 at 24 hr post-dose in plasma valued from 0.671% to 3.94% of Cₘₐₓ. The mean concentration of HBI-2375 at 24 hr post-dose in brain valued from 9.71% to 18.9% of Cmax. The AUC exposure of HBI-2375 in brain was lower than that in plasma, counting as 28.3-33.2% and 36.1-38.8% of that in plasma with single dosing at 40 and 80 mg/kg, and 53, 6-58.8% with repeated dosing at 80 mg/kg/day. The exposure of HBI-2375 in plasma and brain of females were closed to that of males, the ratios were 0.989-1.22 for plasma, and 1.10-1.34 for brain.

Following single-dose oral administration of HBI-2375 at 40 mg/kg and 80 mg/kg in C57BL/6J mice, HBI-2375 in plasma and brain showed a proportional increase of exposure in AUCₗₐₛₜ and Cₘₐₓ following oral dosing proportionally.

Following 7-day repeat oral administration of HBI-2375 at 80 mg/kg in C57BL/6J mice, the AUCₗₐₛₜ and Cₘₐₓ of HBI-2375 in plasma and brain were closed to that from single-dose at 80 mg/kg. This result indicates that there was no obvious drug accumulation in the plasma and brain of C57BL/6J mice after 7-day consecutive dosing of HBI-2375 at 80 mg/kg.

In conclusion, after single oral administration of HBI-2375 at 40 and 80 mg/kg, as well as 7-day repeat oral administration at 80 mg/kg in C57BL/6J mice, HBI-2375 was distributed to brain with Tₘₐₓ at 4 hr and its brain exposure lasted for 24 hours, counting as 28.3-58.8% of AUC in plasma. HBI-2375 in plasma and brain showed a proportional increase of exposure, no gender-related differences and no obvious drug accumulation were observed.

### Example 5: Pharmacokinetics in Humans

A pharmacokinetic profile for a pharmaceutical composition comprising a compound of Formula (I), Formula (II), Formula (III), Formula (Ia), Formula (Ib), Formula (IIIa), or Formula (IIIb) is determined by means analogous to those set forth in the preceeding examples. The compound, or a pharmaceutically acceptable salt thereof, and optionally one or more pharmaceutically acceptable ingredients is provided. The pharmaceutical composition may be administered intravenously (IV), orally (PO), or both. The pharmaceutical composition is administered to one or more human subjects at one or more doses of the compound of from about 1 to about 100 mg per kg of the subject's body weight, e.g. about 5, about 10, about 15, about 20, about 25, about 30, about 35, about 40, about 45, about 50, about 55, about 60, about 65, about 70, about 75, about 80, about 85, about 90, about 95, or about 100 mg per kg of the subject's body weight, or any subsidiary weight within any range formed by two of the foregoing doses. Subjects' blood is drawn immediately prior to drug administration and at timed intervals for 12, 24, 36, or 48 hours post administration. The quantification of compound concentrations may be conducted as per those described above for the in mouse or rat plasma, e.g. by was quantification using LC-MS/MS method. Plasma concentration versus time data may be analyzed by analogous methods. Pharmacokinetic parameters, including C₀ (initial concentration after drug administration), CL (serum drug concentration at steady state), Vass (volume of distribution), Cₘₐₓ (maximum peak concentration of the drug), Tₘₐₓ (time required to reach Cₘₐₓ) T_{1/2} (time required for drug concentration to decrease by 50% after administration), AUC₀₋ₗₐₛₜ (area under the concentration-time curve from dosing time t=0 to the time of the last measured concentration), AUC_{0-inf} (total drug exposure across time), MRT₀₋ₗₐₛₜ (mean residence time from dosing time t=0 to the time of the last measured concentration), MRT_{0-inf} (mean residence time of total drug exposure), bioavailability (%) (the percentage (or the fraction F) of an administered drug dose that reaches the systemic circulation) for one or more groups of subjects may be computed in analogous fashion.

### Example 6: Blood-Brain Barrier Crossing in Humans

The ability of a compound of Formula (IIIb) (HBI_084 (HBI-2375)) to cross the BBB is determined in a population comprising one or more human subjects by orally (PO) or intravenously (IV) administering a pharmaceutical composition comprising a compound of Formula (IIIb) to the individual members of the population of human subjects at one or more doses described in Example 5. Blood samples and CSF are drawn from the subjects and compound concentrations are determined in the blood plasma and CSF of the subjects as described in Example 4. Pharmacokinetic parameters are determined by means analogous to those described in Examples 1-4. Doses for HBI-2375 may be based on a target concentration in subjects' brain predicted using the following table.

TABLE 19
Predicted Brain Concentrations Based
on Mouse/Rat PK/BBB Data

| | HBI-2375 Mean Brain | |
|---|---|---|
| | Concentration (ng/g) | |
| | | Cmax (4 h for |
| | | mouse, |
| | Cmin = | 0.25-1 h |
| Efficacy Dose (mg/kg) | 24 h | for rat) |
| Single PO Mouse at 80 | 55 | 528 |
| Repeat PO Mouse at 80 | 136 | 807 |
| Single PO Rat 40 | 22.1 | 623 |
| Equivalent Human 6.5 | 4.5 | 42.9 |
| (estimated from single PO | | |
| mouse) | | |
| Equivalent Human 6.5 | 11.1 | 65.6 |
| (estimated from repeated | | |
| PO mouse) | | |
| Equivalent Human 6.5 | 3.6 | 100.5 |
| (estimated from sngle PO | | |
| rat) | | |

The compounds disclosed herein have strong inhibitory activity against MLL1-WDR5 protein-protein interaction, can reduce the MLL1 catalytic activity of MLL1 at cellular level, downregulate the expression of Hox and Meis-1 genes and induce apoptosis of leukemia cells. Also, the compounds of the invention exhibit good water solubility, superior bioavailability, blood-brain barrier permeability, and pharmaceutical safety, and can be used for treating a variety of cancers, including leukemia and brain cancer.

The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are incorporated by reference in their entirety for all purposes.

1. A method for treating cancer, the method comprising administering to a patient in need thereof a composition comprising an effective amount of a compound having a structure of Formula (I) or Formula (III): 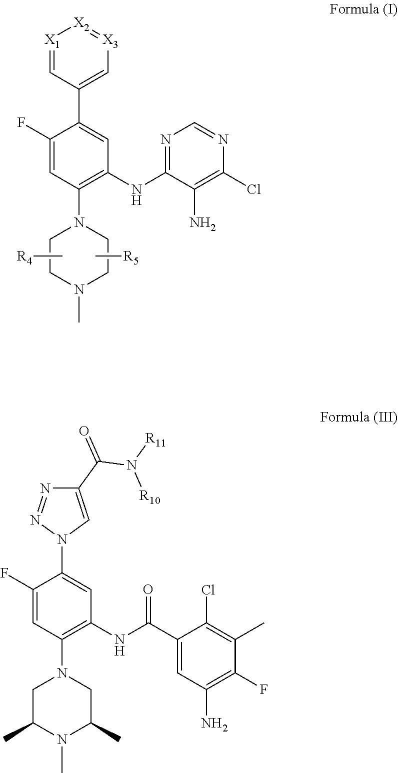
wherein:
X₁ is N or CH;
X₂ is N, CR₂;
X₃ is N or CH;
R₂ is selected from N-morpholino, wherein the morpholino group may be substituted by one or two methyl groups;
each of R₄ and R₅ is hydrogen or alkyl, wherein one or both of R₄ and R₅ may be alkyl;
and each of R₁₀ and R₁₁ is C₁-C₄ alkyl or R₁₀ and R₁₁ together form a 4-alkyl piperazinyl group.

2. A method of claim 1, wherein the compound has a structure selected from the group consisting of a Formula (Ia), Formula (Ib), Formula (IIIa) and Formula (IIIb), or a pharmaceutically acceptable salt, polymorph, solvate, enantiomer, stereoisomer, or prodrug thereof:

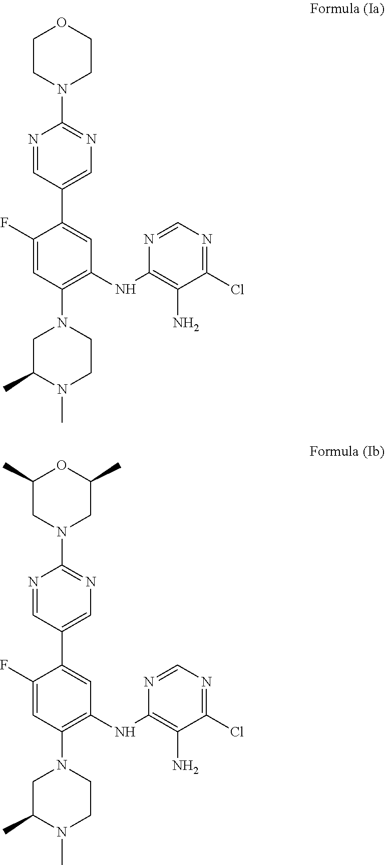

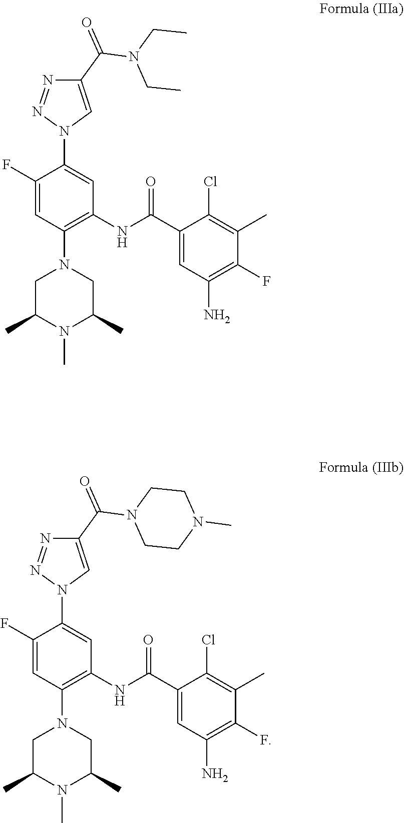 [SMILES: CCN(CC)C(=O)c1cn(-c2cc(NC(=O)c3cc(N)c(F)c(C)c3Cl)c(N3C[C@@H](C)N(C)[C@@H](C)C3)cc2F)nn1 ; Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

3. The method of claim 1, wherein the compound has a structure of Formula (Ia), or a pharmaceutically acceptable salt, polymorph, solvate, enantiomer, stereoisomer, or prodrug thereof.

4. The method of claim 1, wherein the compound has a structure of Formula (Ib), or a pharmaceutically acceptable salt, polymorph, solvate, enantiomer, stereoisomer, or prodrug thereof.

5. The method of claim 1, wherein the compound has a structure of Formula (IIIa), or a pharmaceutically acceptable salt, polymorph, solvate, enantiomer, stereoisomer, or prodrug thereof.

6. The method of claim 1, wherein the compound has a structure of Formula (IIIb), or a pharmaceutically acceptable salt, polymorph, solvate, enantiomer, stereoisomer, or prodrug thereof.

7. The method of claim 1, wherein the compound has a structure of Formula (II) or a pharmaceutically acceptable salt, polymorph, solvate, enantiomer, stereoisomer, or prodrug thereof: 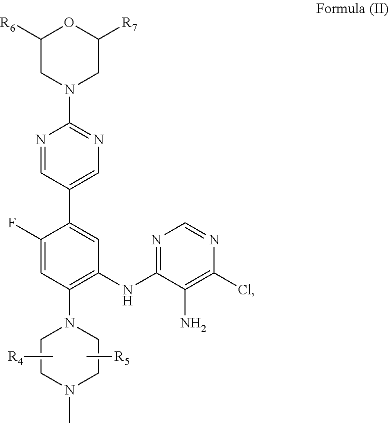
wherein:
each of R₄ and R₅ is hydrogen or methyl, wherein at least one of R₅ and R₆ is methyl, and
each of R₆ and R₇ is hydrogen or methyl.

8. The method of claim 1, comprising oral administration of an effective amount of the compound to the patient.

9. The method of claim 8, wherein the effective amount of the compound is about 0.1 to about 1000 mg per kg of the patient's body weight (mpk).

10. The method of claim 9, wherein the effective amount of the compound is about 1 to about 100 mg per kg of the patient's body weight (mpk).

11. The method of claim 10, wherein the effective amount of the compound is about 1 to about 20 mg per kg of the patient's body weight (mpk).

12. The method of claim 8,
wherein the patient is a human and the compound is administered at a dose of about 1 to about 20 mg per kg of the patient's body weight.

13. The compound of claim 12, wherein when the compound is administered to a human at a dose of about 1 to about 20 mg per kg of the patient's body weight, a brain concentration Cₘₐₓ of about 10 to about 310 ng per gram of the patient's estimated brain weight.

14. The method of claim 1, comprising administering the compound once per day (Q.D.), once every other day (Q.O.D.), every week (Q.W.), two times per week (BIW) or three times per week (TIW).

15. The method of claim 1, comprising administering the compound on a dosing schedule, wherein the dosing schedule comprises:
a. a first dosing period comprising from about one cycle of administration of the effective dose of the compound at a frequency of from about once per week (Q.W.);
b. a first drug holiday; and
c. a second dosing period comprising from about one cycle of administration of the effective dose of the compound at a frequency of from about once per week (Q.W.).

16. The method of claim 15, wherein each cycle is from about seven days or from about twelve days long.

17. The method of claim 15, wherein the drug holiday is from about as long as each cycle.

18. The method of claim 1, comprising administering the compound on a dosing schedule, wherein the dosing schedule comprises:
a. a first dosing period comprising from about one cycle of administration of the effective dose of the compound at a frequency of from about once per week (Q.W.);
b. a first drug holiday.

19. The method of claim 18, wherein the drug holiday is from about as long as about one cycle.

20. The method of claim 19, wherein the drug holiday is from about one month, one to six months, or one to twelve months.

21. The method of claim 4,
wherein the compound, when administered orally to a dog in an amount of 1.5 mg per kilogram of the dog's body weight (mpk), results in a maximum blood plasma concentration (Cmax) in the dog of from about 150 to about 235 nM.

22. The method of claim 4,
wherein the compound, when administered orally to a dog in an amount of 1.5 mg per kilogram of the dog's body weight (mpk), the area under the area under the blood plasma concentration curve (AUC₀₋ₗₐₛₜ) is from about 4225 nM·h to about 6603 nM·h.

23. The method of claim 4,
wherein the compound, when administered orally to a dog in an amount of 4.5 mg per kilogram of the dog's body weight (mpk), results in a maximum blood plasma concentration (Cmax) in the dog of from about 356 nM to about 556 nM.

24. The method of claim 4,
wherein the compound, when administered orally to a dog in an amount of 4.5 mg per kilogram of the dog's body weight (mpk), the area under the area under the blood plasma concentration curve (AUC₀₋ₗₐₛₜ) is from about 10734 nM·h to about 16772 nM·h.

25. The method of claim 4,
wherein the compound, when administered orally to a mouse in an amount of 10 mg per kilogram of the mouse's body weight (mpk), results in a maximum blood plasma concentration (Cmax) in the mouse of from about 1056 to about 1651 nM.

26. The method of claim 4,
wherein the compound, when administered orally to a mouse in an amount of 10 mg per kilogram of the mouse's body weight (mpk), the area under the area under the blood plasma concentration curve (AUC₀₋ₗₐₛₜ) is from about 13132 nM·h to about 20519 nM·h.

27. The method of claim 4,
wherein the compound, when administered orally to a mouse in an amount of 30 mg per kilogram of the mouse's body weight (mpk), results in a maximum blood plasma concentration (Cmax) in the mouse of from about 2744 to about 4288 nM.

28. The method of claim 4,
wherein the compound, when administered orally to a mouse in an amount of 30 mg per kilogram of the mouse's body weight (mpk), the area under the area under the blood plasma concentration curve (AUC₀₋ₗₐₛₜ) is from about 44791 nM·h to about 69986 nM·h.

29. (canceled)

30. The method of claim 3,
wherein the compound, when administered orally to a mouse in an amount of 10 mg per kilogram of the mouse's body weight (mpk), results in a maximum blood plasma concentration (Cmax) in the mouse of from about 1338 to about 2091 nM.

31. The method of claim 3,
wherein the compound, when administered orally to a mouse in an amount of 10 mg per kilogram of the mouse's body weight (mpk), the area under the area under the blood plasma concentration curve (AUC₀₋ₗₐₛₜ) is from about 5681 nM·h to about 8876 nM·h.

32. The method of claim 3,
wherein the compound, when administered orally to a mouse in an amount of 30 mg per kilogram of the mouse's body weight (mpk), results in a maximum blood plasma concentration (Cmax) in the mouse of from about 4374 to about 6834 nM.

33. The method of claim 3,
wherein the compound, when administered orally to a mouse in an amount of 30 mg per kilogram of the mouse's body weight (mpk), the area under the area under the blood plasma concentration curve (AUC₀₋ₗₐₛₜ) is from about 29637 nM·h to about 46309 nM·h.

34. (canceled)

35. The method of claim 6,
wherein the compound, when administered orally to a dog in an amount of 1.5 mg per kilogram of the dog's body weight (mpk), results in a maximum blood plasma concentration (Cmax) in the dog of from about 6.7 nM to about 11 nM.

36. The method of claim 6,
wherein the compound, when administered orally to a dog in an amount of 1.5 mg per kilogram of the dog's body weight (mpk), the area under the area under the blood plasma concentration curve (AUC₀₋ₗₐₛₜ) is from about 37 nM·h to about 59 nM·h.

37. The method of claim 6,
wherein the compound, when administered orally to a dog in an amount of 4.5 mg per kilogram of the dog's body weight (mpk), results in a maximum blood plasma concentration (Cmax) in the dog of from about 38 nM to about 60 nM.

38. The method of claim 6,
wherein the compound, when administered orally to a dog in an amount of 4.5 mg per kilogram of the dog's body weight (mpk), the area under the area under the blood plasma concentration curve (AUC₀₋ₗₐₛₜ) is from about 367 nM·h to about 574 nM·h.

39. The method of claim 6,
wherein the compound, when administered orally to a mouse in an amount of 10 mg per kilogram of the mouse's body weight (mpk), results in a maximum blood plasma concentration (Cmax) in the mouse of from about 287 to about 449 nM.

40. The method of claim 6,
wherein the compound, when administered orally to a mouse in an amount of 10 mg per kilogram of the mouse's body weight (mpk), the area under the area under the blood plasma concentration curve (AUC₀₋ₗₐₛₜ) is from about 1050 nM·h to about 1641 nM·h.

41. The method of claim 6,
wherein the compound, when administered orally to a mouse in an amount of 30 mg per kilogram of the mouse's body weight (mpk), results in a maximum blood plasma concentration (Cmax) in the mouse of from about 1020 to about 1595 nM.

42. The method of claim 6,
wherein the compound, when administered orally to a mouse in an amount of 30 mg per kilogram of the mouse's body weight (mpk), the area under the area under the blood plasma concentration curve (AUC₀₋ₗₐₛₜ) is from about 4515 nM·h to about 7055 nM·h.

43. (canceled)

44. The method of claim 5,
wherein the compound, when administered orally to a mouse in an amount of 10 mg per kilogram of the mouse's body weight (mpk), results in a maximum blood plasma concentration (Cmax) in the mouse of from about 1337 to about 2090 nM.

45. The method of claim 5,
wherein the compound, when administered orally to a mouse in an amount of 10 mg per kilogram of the mouse's body weight (mpk), the area under the area under the blood plasma concentration curve (AUC₀₋ₗₐₛₜ) is from about 1779 nM·h to about 2780 nM·h.

46. The method of claim 5,
wherein the compound, when administered orally to a mouse in an amount of 30 mg per kilogram of the mouse's body weight (mpk), results in a maximum blood plasma concentration (Cmax) in the mouse of from about 3627 to about 5668 nM.

47. The method of claim 5,
wherein the compound, when administered orally to a mouse in an amount of 30 mg per kilogram of the mouse's body weight (mpk), the area under the area under the blood plasma concentration curve (AUC₀₋ₗₐₛₜ) is from about 6084 nM·h to about 9506 nM·h.

48. (canceled)

49. The method of claim 1, wherein the compound passes through the blood brain barrier.

50. The method of claim 1, where the patient has a brain tumor.

51. The method of claim 50, wherein the brain tumor is a metastatic brain tumor, a meningioma, a neuroblastoma, a glioblastoma, or an astrocytoma.

52. A method for treating cancer, the method comprising administering to a patient in need thereof a composition comprising an effective amount of a compound having a structure of Formula (IIIb):

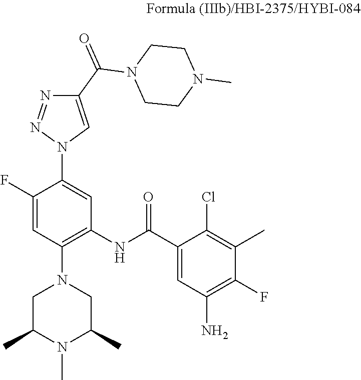 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

53. The method of claim 52, wherein the compound passes through the blood brain barrier.

54. The method of claim 52, wherein the cancer is a solid cancer, hematological cancer or brain cancer.

55. The method of claim 52, wherein the effective amount of the compound is about 0.5 to about 20 mg per kg of the patient's body weight (mpk).

56. The method of claim 1,
wherein the patient is a human and the compound is administered at a dose of about 1 to about 20 mg per kg of the patient's body weight.

57. The method of claim 56, wherein when the compound is administered to a human at a dose of about 1 to about 20 mg per kg of the patient's body weight, a brain concentration Cₘₐₓ of about 10 to about 310 ng per gram of the patient's estimated brain weight.

58. The method of claim 52, wherein the compound, when administered orally to a mouse in an amount of 40 mg per kilogram of the mouse's body weight (mpk), results in a maximum blood plasma concentration (Cmax) in the mouse of from about 770 ng/mL to about 1505 ng/mL in about 30 minutes.

59. The method of claim 52, wherein the compound, when administered orally to a mouse in an amount of 40 mg per kilogram of the mouse's body weight (mpk), results in a mean concentration in the mouse brain of from about 58 ng/mL to about 156 ng/mL in about 30 minutes.

60. The method of claim 52, wherein the compound, when administered orally to a mouse in an amount of 80 mg per kilogram of the mouse's body weight (mpk), results in a maximum blood plasma concentration (Cmax) in the mouse of from about 2700 ng/mL to about 5200 ng/mL in about 30 minutes.

61. The method of claim 52, wherein the compound, when administered orally to a mouse in an amount of 80 mg per kilogram of the mouse's body weight (mpk), results in a mean concentration in the mouse brain of from about 191 ng/mL to about 540 ng/mL in about 30 minutes.

62. The method of claim 52, wherein the compound, when administered orally to a mouse in an amount of 40 mg per kilogram of the mouse's body weight (mpk), results in a maximum concentration (Cmax) in the mouse brain of from about 126 ng/mL to about 234 ng/mL in about 4 hours.

63. The method of claim 52, wherein the compound, when administered orally to a mouse in an amount of 80 mg per kilogram of the mouse's body weight (mpk), results in a maximum concentration (Cmax) in the mouse brain of from about 360 ng/mL to about 700 ng/mL in about 4 hours.

64. The method of claim 52, wherein the compound, when administered orally to a mouse in an amount of 40 mg per kilogram of the mouse's body weight (mpk), the area under the blood plasma concentration curve (AUCₗₐₛₜ) is from about 4600 hr·ng/mL to about 8600 hr-ng/mL.

65. The method of claim 52, wherein the compound, when administered orally to a mouse in an amount of 40 mg per kilogram of the mouse's body weight (mpk), the area under the brain concentration curve (AUCₗₐₛₜ) is from about 1400 hr·ng/mL to about 2640 hr·ng/mL.

66. The method of claim 52, wherein the compound, when administered orally to a mouse in an amount of 80 mg per kilogram of the mouse's body weight (mpk), the area under the blood plasma concentration curve (AUCₗₐₛₜ) is from about 10900 hr·ng/mL to about 20200 hr·ng/mL.

67. The method of claim 52, wherein the compound, when administered orally to a mouse in an amount of 80 mg per kilogram of the mouse's body weight (mpk), the area under the brain concentration curve (AUCₗₐₛₜ) is from about 4090 hr·ng/mL to about 7600 hr·ng/mL.

68. The method of claims any on claim 52, wherein the compound, when administered orally to a dog in an amount of 1.5 mg per kilogram of the dog's body weight (mpk), results in a maximum blood plasma concentration (Cmax) in the dog of from about 150 to about 235 nM.

69. The method of claims any on claim 52, wherein the compound, when administered orally to a dog in an amount of 1.5 mg per kilogram of the dog's body weight (mpk), the area under the area under the blood plasma concentration curve (AUC₀₋ₗₐₛₜ) is from about 4225 nM·h to about 6603 nM·h.

70. A compound that inhibits MLL1-WDR5 protein-protein interactions and crosses the blood-brain barrier.

71. The compound of claim 70, wherein the compound's structure is Formula (I) or Formula (III). 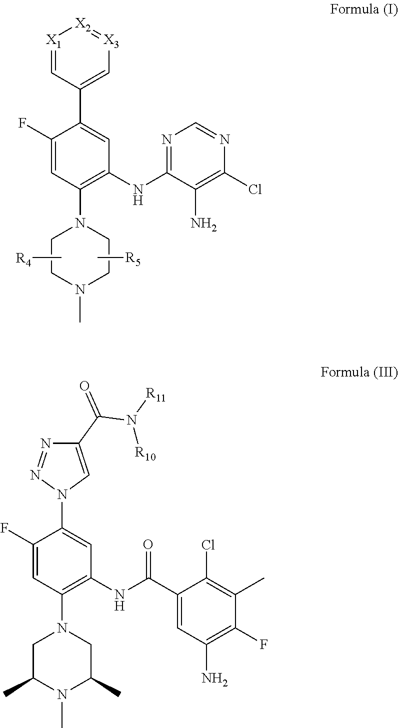
wherein:
X₁ is N or CH;
X₂ is N, CR₂;
X₃ is N or CH;
R₂ is selected from N-morpholino, wherein the morpholino group may be substituted by one or two methyl groups;
each of R₄ and R₅ is hydrogen or alkyl, wherein one or both of R₄ and R₅ may be alkyl;
and each of R₁₀ and R₁₁ is C₁-C₄ alkyl or R₁₀ and R₁₁ together form a 4-alkyl piperazinyl group.

72. The compound of claim 70, wherein the compound has a structure selected from the group consisting of a Formula (Ia), Formula (Ib), Formula (IIIa) and Formula (IIIb), or a pharmaceutically acceptable salt, polymorph, solvate, enantiomer, stereoisomer, or prodrug thereof.

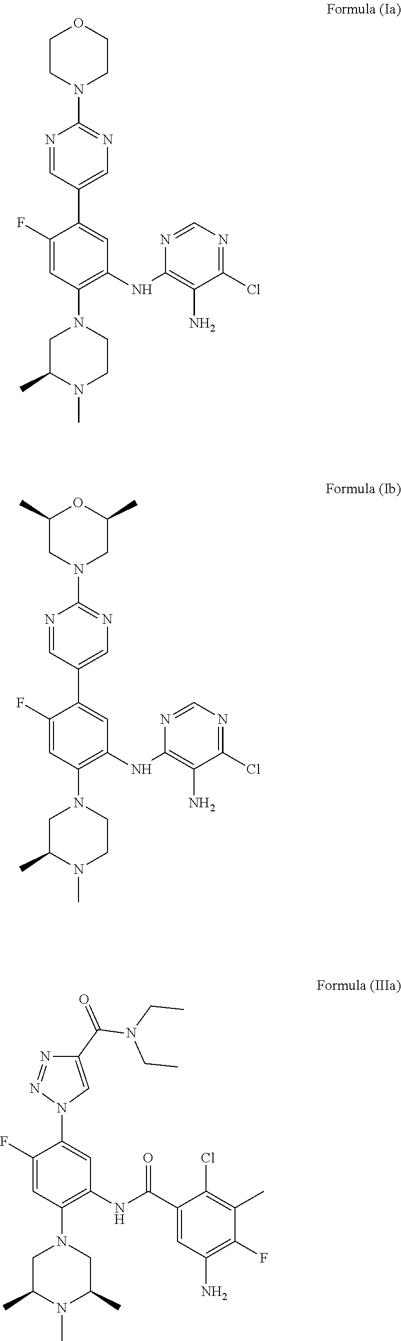 [SMILES: C[C@H]1CN(c2cc(F)c(-c3cnc(N4CCOCC4)nc3)cc2Nc2ncnc(Cl)c2N)CCN1C ; C[C@@H]1CN(c2ncc(-c3cc(Nc4ncnc(Cl)c4N)c(N4CCN(C)[C@@H](C)C4)cc3F)cn2)C[C@H](C)O1 ; CCN(CC)C(=O)c1cn(-c2cc(NC(=O)c3cc(N)c(F)c(C)c3Cl)c(N3C[C@@H](C)N(C)[C@@H](C)C3)cc2F)nn1]

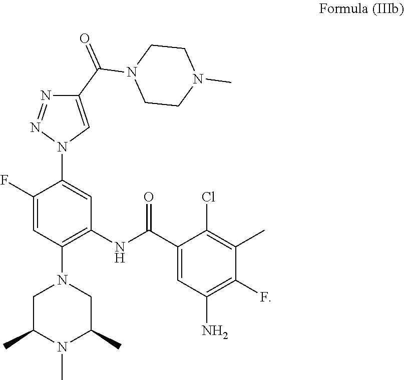 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

73. The compound of claim 72, having the structure of Formula (IIIb).

74. The compound of claim 70 for use in the treatment of cancer.

75. The compound of claim 74, wherein the cancer to be treated is a brain cancer.

76. The compound of claim 75, wherein the cancer to be treated is a primary brain tumor or a secondary brain tumor.

77. The compound of claim 76, wherein the cancer to be treated is a metastatic tumor.

78. The compound of claim 77, wherein the cancer to be treated is a glioblastoma, a neuroblastoma, or an astrocytoma.

79. A pharmaceutical composition comprising the compounds of claim 72 and optionally one or more pharmaceutically acceptable ingredients.

80. Use of the compounds of claim 72 for preparation of a pharmaceutical composition.

81. The use of claim 80, wherein the pharmaceutical composition comprises one or more pharmaceutically acceptable ingredients.

82. A process of making a pharmaceutical composition of claim 81, comprising admixing the compound with one or more pharmaceutically acceptable ingredients.
